(19) United States
(12) Patent Application Publication
(10) Pub. No.: US 2025/0135134 A1
(43) Pub. Date: May 1, 2025

(54) DATA CAPTURE, PROCESSING, STORAGE AND RENDERING SYSTEM FOR BREATHING ASSISTANCE APPARATUS

(71) Applicant: Fisher & Paykel Healthcare Limited, Auckland (NZ)

(72) Inventors: Christopher Harding CAMPBELL, Auckland (NZ); Pauline Joy Alfonso ELLOSO, Auckland (NZ); James Alexander Michael REVIE, Auckland (NZ); Robert Stuart KIRTON, Auckland (NZ); Benjamin Wilson CASSE, Auckland (NZ); Kevin Peter O'DONNELL, Auckland (NZ); Alberto Antonio ZAFFARONI, Auckland (NZ); David Martin RUSSELL, Auckland (NZ)

(21) Appl. No.: 18/707,100

(22) PCT Filed: Nov. 15, 2022
(86) PCT No.: PCT/IB2022/060951

§ 371 Date: May 2, 2024

Related U.S. Application Data
(60) Provisional application No. 63/279,575, filed on Nov. 15, 2021.

(51) Int. Cl. A61M 16/00; A61B 5/00; A61M 16/16; G16H 15/00

(52) U.S. Cl. CPC A61M 16/024; A61B 5/743; A61M 16/16; G16H 15/00; A61M 2205/502

(57) ABSTRACT

A breathing assistance apparatus and related systems for data capture, processing, storage and rendering. The data may related to a personal health enquiry presented to a patient of a breathing assistance apparatus on a display screen of the apparatus. The responses to a set of queries in the enquiry may be captured as answer data. The answer data may then be transmitted and/or processed relative to baseline data for access and rendering as one or more data reports and/or time-series charts.

### FIELD OF THE DISCLOSURE

This disclosure relates to a breathing assistance apparatus and related systems for data capture, processing, storage and rendering.

### BACKGROUND

Breathing assistance apparatuses are used in various environments such as hospital, medical facility, residential care, or home environments to deliver a flow of gases to users or patients. A breathing assistance or respiratory therapy apparatus may be used to deliver supplementary oxygen or other gases with a flow of gases, and/or may include a humidification apparatus to deliver heated and humidified gases. A breathing assistance apparatus may allow adjustment and control over characteristics of the gases flow, including flow rate, temperature, gases concentration, humidity, pressure, etc. Sensors, such as flow sensors and/or pressure sensors are used to measure characteristics of the gases flow.

### SUMMARY

In an aspect, this disclosure broadly comprises a system comprising: a respiratory therapy device that is configured to deliver a flow of gases to a user for respiratory therapy comprising: a flow generator for generating the flow of gases, a humidifier for heating and humidifying the flow of gases, a controller that is operable to control the flow generator and humidifier, a user interface comprising a display to enable user interaction with the controller; a data server that is in data communication with the respiratory therapy device; and a display device that is in data communication with the data server, the controller of the respiratory therapy device being configured to receive answer data based on user input to the user interface to a set of presented queries on the display relating to one or more health parameters from at least one of a symptoms category and a medication category, and send the answer data to the data server, the data server being configured to receive the answer data from the respiratory therapy device, process the answer data relative to baseline data associated with the user to generate a data report that is accessible or rendered for display by the display device, and wherein: the data report comprising a colour-coded time-series plot of the answer data for a user for the one or more health parameters over a time period, and wherein each plotted answer of the answer data is selectively colour-coded for display according to a baseline colour spectrum or one or more deviation colour spectrums, the colour spectrum selected for each answer being a function of or based on any deviation of the answer data relative to the baseline data, and the generated data report being displayed or rendered on the display device.

In a configuration, the baseline colour spectrum is visually different or distinctive to the one or more deviation colour spectrums.

In a configuration, the baseline colour spectrum has a high visual contrast to the one or more deviation colour spectrums.

In a configuration, the time-series plot comprises a worsening deviation colour spectrum that represents a worsening or deterioration of the health parameters represented by the answer data relative to the baseline data.

In a configuration, any answer data corresponding to or matching the baseline data is represented as a plotted answer that is colour-coded according to the baseline colour spectrum, and any answer data that represents a worsening deviation from the baseline data is represented as a plotted answer that is colour-coded according to the worsening deviation colour spectrum.

In a configuration, the time-series plot further comprises an improvement deviation colour spectrum that represents an improvement of the health parameters represented by the answer data relative to the baseline data.

In a configuration, any answer data corresponding to an improvement from the baseline data is represented as a plotted answer that is colour-coded according to the improvement deviation colour spectrum.

In a configuration, the answer data for each health parameter query comprises or is convertible to a score or rating on a scale, each score or rating on the scale corresponding to or being assigned a respective discrete colour value on each of the baseline colour spectrum and the one or more deviation colour spectrums such that each plotted answer of the answer data is assigned the discrete colour value from the selected colour spectrum that is associated with its score.

In a configuration, the score is numerical, and the scale extends from a desirable score or rating at one end to a non-desirable score or rating at the other end of the scale.

In a configuration, each of the baseline colour spectrum and the one or more deviation colour spectrums comprises a range of discrete colour values of a base or dominant colour extending from lighter colour values at one end of the spectrum to darker colour values at the other end of the spectrum.

In a configuration, desirable scores or ratings on the scale correspond to or are assigned discrete colour values on the baseline colour spectrum and one or more deviation colour spectrums that are lighter compared to non-desirable scores or ratings, or vice versa, or wherein one or more of the colour spectrums are configured such that desirable scores or ratings on the scale correspond to or are assigned discrete colour values that are lighter compared to non-desirable scores or ratings and one or more of the other colour spectrums are configured such that desirable scores or ratings on the scale correspond to or are assigned discrete colour values that are darker compared to non-desirable scores.

In a configuration, the base or dominant colour associated with each respective baseline colour spectrum and the one or more deviation colour spectrums comprises a colour, hue, tint, tone, or shade.

In a configuration, the base or dominant colour associated with the baseline colour spectrum is a cool colour, hue, tint, tone or shade, and the base or dominant colour of a worsening deviation colour spectrum is a warm colour, hue, tint, tone or shade.

In a configuration, each plotted answer is colour coded according to a colour spectrum that is selected dependent on whether the answer data corresponds to or deviates from the baseline data, and colour coded within the selected colour spectrum based on an assigned colour value that corresponds to or is a function of the desirability or position of a score or rating associated with the answer data relative to a scale.

In a configuration, the answer data for each health parameter query comprises or is convertible to a score or rating on a scale, each score or rating on the scale corresponding to or being assigned a respective discrete colour value on each of the baseline colour spectrum and the one or more deviation colour spectrums such that each plotted answer of the answer data is assigned the discrete colour value from the selected colour spectrum that is associated with its score.

In a configuration, the discrete colour values in each colour spectrum represent a discrete score or rating on the scale, the scale defined by lighter colour values at one end representing desirable scores or ratings to darker colour values representing non-desirable scores or ratings, or vice versa, or wherein the scale for one or more of the colour spectrums is defined by lighter colour values at one end representing desirable scores or ratings to darker colour values representing non-desirable scores or ratings and the scale for one or more of the other colour spectrums is defined by darker colour values at one end representing desirable scores or ratings to lighter colour values representing non-desirable scores or ratings.

In a configuration, the baseline colour spectrum and one or more deviation colour spectrums are each represented in the data report visually by respective colour keys that depict the discrete colour values of each colour spectrum with respect to their position on the scale associated with the answer data.

In a configuration, the plotted answers are plotted in columns according to a time-interval frequency along the time-axis of the plot and rows according to the one or more health parameters to which the answers relate.

In a configuration, the time-interval frequency is daily such that each column of answer data is associated with a specific day within a time period spanning a number of days. In another configuration, the time-interval frequency is weekly such that each column of answer data is associated with a specific week within a time period spanning a number of weeks.

In a configuration, the answer data has associated time-interval data representing a time interval the answer data was input by the user, and wherein the answers are plotted in an array, stack, or column along the time-axis of the time-series plot according to their respective time interval, and with the answers across time intervals being aligned in rows according to their associated health parameter.

In a configuration, each plotted answer is represented by a colour-coded data marker in the plot of any shape or size.

In a configuration, the data report generated comprises a plurality of distinct colour-coded time-series plots relating to a plurality of different users that are presented or displayed together.

In a configuration, the display of the user interface comprises a touchscreen display providing a graphical user interface (GUI), and wherein the user input is provided via the touchscreen display and/or one or more input elements.

In a configuration, the display of the user interface presents a graphical user interface (GUI) and the user interface comprises one or more input elements that are operable by a user to provide user input to the GUI.

5 In a configuration, the presented queries on the display relate to one or more health parameters from a symptoms category or a medication category, or at least one health parameter from each category.

In an aspect, this disclosure broadly comprises a method of generating a data report for display, the method implemented by an electronic device comprising a processor having associated memory, and comprising: receiving or retrieving answer data into memory, the answer data representing user input to a set of presented queries relating to one or more health parameters from at least one of a symptoms category and a medication category for a user of a respiratory therapy device; receiving or retrieving baseline data into memory, the baseline data associated with the user; processing the answer data relative to the baseline data to generate a data report that is accessible or rendered for display on a display device associated with or connected to the electronic device, and wherein: the data report comprises a colour-coded time-series plot of the answer data for a user for the one or more health parameters over a time period, and wherein each plotted answer of the answer data is selectively colour-coded for display according to a baseline colour spectrum or one or more deviation colour spectrums, the colour spectrum selected for each answer being a function of or based on any deviation of the answer data relative to the baseline data; and displaying the generated data report on the display device.

In a configuration, the electronic device is a computing device that is configured to receive the answer data from a data server and/or from a respiratory therapy device.

In a configuration, the electronic device is a data server that is configured to receive the answer data from a respiratory therapy device directly over a data communication network or indirectly via another electronic device over a data communication network.

In a configuration, the electronic device is a respiratory therapy device that is configured to deliver a flow of gases to a user for respiratory therapy comprising: a flow generator for generating the flow of gases, a humidifier for heating and humidifying the flow of gases, a controller that is operable to control the flow generator and humidifier, a touchscreen providing a graphical user interface (GUI) to enable user interaction with the controller; and the controller of the respiratory therapy device being configured to receive answer data based on user input to the touchscreen GUI to a set of presented queries relating to one or more health parameters.

In an aspect, this disclosure broadly comprises a software application for generating a data report, the application comprising a set of instructions for execution by a processor and associated memory of an electronic device to implement the steps of: receiving or retrieving answer data into memory, the answer data representing user input to a set of presented queries relating to one or more health parameters from at least one of a symptoms category and a medication category; receiving or retrieving baseline data into memory, the baseline data associated with the user; processing the answer data relative to the baseline data to generate a data report comprising a colour-coded time-series plot of the answer data for a user for the one or more health parameters over a time period, and wherein each plotted answer of the answer data is selectively colour-coded for display according to a baseline colour spectrum or one or more deviation colour spectrums, the colour spectrum selected for each answer being a function of or based on any deviation of the answer data relative to the baseline data; and outputting the data report as a data file for rendering on a display or rendering the data report on an associated display.

In a configuration, the software application further comprising: receiving a data request to view a data report associated with one or more users; and generating and rendering the data report on a display in response to the data request.

In an aspect, this disclosure broadly comprises a graphical user interface (GUI) and processor for displaying a data report, the GUI being controlled by the processor to render the data report on the GUI, the processor being configured to: retrieve or receive report data representing the data report into memory, the report data comprising: answer data representing user input to a set of presented queries relating to one or more health parameters from at least one of a symptoms category and a medication category, and baseline data associated with the user; process the report data in accordance with rendering instructions; and render the data report on the GUI based on the report data and in accordance with the rendering instructions, the rendering instructions being configured to render a data report that comprises a colour-coded time-series plot of the answer data for a user for the one or more health parameters over a time period, and wherein each plotted answer of the answer data is selectively colour-coded for display according to a baseline colour spectrum or one or more deviation colour spectrums, the colour spectrum selected for each answer being a function of or based on any deviation of the answer data relative to the baseline data.

In an aspect, this disclosure broadly comprises a system comprising: multiple respiratory therapy devices that are configured to deliver a flow of gases to respective multiple users for respiratory therapy, each respiratory therapy device comprising: a flow generator for generating the flow of gases, a humidifier for heating and humidifying the flow of gases, a controller that is operable to control the flow generator and humidifier, a user interface comprising a display to enable user interaction with the controller; a data server that is in data communication with the respiratory therapy devices; and a display device that is in data communication with the data server, the controllers of the respiratory therapy devices being configured to receive answer data comprising symptom data and/or medication data based on user input to the user interface to a set of presented queries on the display relating to one or more health parameters from at least one of a symptoms category and a medication category, and send the answer data to the data server, the data server being configured to receive the answer data from the multiple respiratory therapy devices for the multiple users, process the answer data relative to baseline data associated with each respective user to generate a graphical dashboard that is accessible or rendered for display by the display device, and wherein: the graphical dashboard depicts a summary time-series chart for each user of their processed answer data relative to their baseline data on a category basis, each summary time-series chart depicting or representing deviations of the symptom data and/or medication data relative to their respective baseline data over a time period, and the generated graphical dashboard being displayed or rendered on the display device.

In a configuration, the summary time-series chart for each user comprises a time-series graph that plots both symptom data deviation and medication data deviation.

In a configuration, the plots of symptom data deviation and medication data deviation are visually distinguishable from each other or visually represented as separate data sets within the same time-series graph.

In a configuration, the plots of symptom data deviation and medication data deviation are represented by the same or different types of plots.

In a configuration, the plots of symptom data deviation and medication data deviation are represented by independent plots within the same time-series graph or as combined plots within the same time-series graph.

In a configuration, the plots representing the symptom data deviation and medication data deviation are selected from any one or more of the following types of plots: dot plot, line chart or line graph, bar chart, lollipop chart, stacked bar chart, and grouped bar chart.

In a configuration, the summary time-series graph for each user comprises the symptom data deviation being plotted as a time-series bar chart and the medication data deviation being plotted as a time-series line chart, or vice versa, within the same time-series graph.

In a configuration, the bar and line charts representing the symptom data deviation and medication data deviation are plotted relative to the same deviation axis such that they overlay each other visually.

In a configuration, the bar and line charts representing the symptom data deviation and medication data deviation are visually distinguished from each other by colour, pattern, or shade.

In a configuration, the data server is configured to process the answer data to extract or generate symptom data deviation and medication data deviation in the form of category deviation parameters for plotting that represent the total number of health parameters in each respective category for a user that have deviated from their respective baseline data for each time-interval associated with the time-axis of the time-series graph.

In a configuration, the data server is configured to generate: a symptom category deviation parameter representing or being a function of the total number of health parameters that deviate from their baseline for each time-interval, and a medication category deviation parameter representing or being a function of the total number of health parameters that deviate from their baseline for each time-interval, each deviation parameter being plotted in the time-series graph; or a worsening deviation parameter for each category representing or being a function of the total number of health parameters in the category that have a worsening deviation from their baseline for each time-interval, and an improvement deviation parameter for each category representing or being a function of the total number of health parameters in the category that have an improving deviation from their baseline for each time-interval, each deviation parameter being plotted in the time-series graph.

In a configuration, the data server is configured to generate the category deviation parameters for each time-interval as a function of or based on a colour-coding of the answer data, wherein the answer data is colour-coded according to either a baseline colour spectrum or a deviation colour spectrum depending on whether the answer deviates from baseline data for the user.

In a configuration, the data server is configured to generate the symptom category deviation parameter(s) and medication category deviation parameter(s) for each time-interval based on a data set representing a related colour-coded time-series plot of the answer data for the user for the one or more health parameters over a time period, wherein each plotted answer of the answer data is selectively colour-coded for display according to a baseline colour spectrum or one or more deviation colour spectrums, the colour spectrum selected for each answer being a function of or based on any deviation of the answer data relative to the baseline data.

In a configuration, the category deviation parameters for each time-interval are based on or are a function of the number of answers in each respective category that are colour coded into a respective one of the deviation colour spectrums.

In a configuration, the summary time-series chart for each user comprises a colour-coded time-series plot of the answer data for the user for the one or more health parameters over a time period, and wherein each plotted answer of the answer data is selectively colour-coded for display according to a baseline colour spectrum or one or more deviation colour spectrums, the colour spectrum selected for each answer being a function of or based on any deviation of the answer data relative to the baseline data.

In a configuration, the plotted answers are grouped in rows or otherwise aligned with their associated health parameter when plotted against the time-axis of the colour-coded time-series plot.

In a configuration, the plotted answers are presented in defined category groups based on the category associated with the health parameter to which the plotted answer relates.

In a configuration, the data server is further configured to process the answer data for the multiple users to determine a priority status between the users, and wherein the graphical dashboard generated is configured to display or present the summary time-series charts of the multiple users based at least partly on the determined priority status of the users.

In a configuration, the priority status of each user represents their general health status relative to the other users based on the answer data, and wherein the graphical dashboard is configured such that the summary time-series charts are presented in an order or arrangement that prioritises the users having a worse health status according to their priority status.

In a configuration, the display of the user interface comprises a touchscreen display providing a graphical user interface (GUI), and wherein the user input is provided via the touchscreen display and/or one or more input elements.

In a configuration, the display of the user interface presents a graphical user interface (GUI) and the user interface comprises one or more input elements that are operable by a user to provide user input to the GUI.

In a configuration, the presented queries on the display relate to one or more health parameters from a symptoms category or a medication category, or at least one health parameter from each category.

In an aspect, this disclosure broadly comprises a method of generating a graphical dashboard for display, the method implemented by an electronic device comprising a processor having associated memory, and comprising: receiving or retrieving answer data into memory, the answer data comprising symptom data and/or medication data representing user input to a set of presented queries relating to one or more health parameters from at least one of a symptoms category and a medication category, the answer data relating to multiple users of respective respiratory therapy devices; receiving or retrieving baseline data into memory, the baseline data associated with each respective user of the multiple users; processing, for each user, the answer data relative to the baseline data to generate a graphical dashboard that is accessible or rendered for display on a display device associated with or connected to the electronic device, and wherein: the graphical dashboard depicts a summary time-series chart for each user of their processed answer data relative to their baseline data on a category basis, each summary time-series chart depicting or representing deviations of the symptom data and/or medication data relative to their respective baseline data over a time period; and displaying the generated graphical dashboard on the display device.

In a configuration, the electronic device is a computing device that is configured to receive the answer data from a data server and/or from multiple respiratory therapy devices.

In a configuration, the electronic device is a data server that is configured to receive the answer data from multiple respiratory therapy devices directly over a data communication network or indirectly via another electronic device over a data communication network.

In an aspect, this disclosure broadly comprises a software application for generating a graphical dashboard, the application comprising a set of instructions for execution by a processor and associated memory of an electronic device to implement the steps of: receiving or retrieving answer data into memory, the answer data comprising symptom data and/or medication data representing user input to a set of presented queries relating to one or more health parameters from at least one of a symptoms category and a medication category, the answer data relating to multiple users of respective respiratory therapy devices; receiving or retrieving baseline data into memory, the baseline data associated with each respective user of the multiple users; processing, for each user, the answer data relative to the baseline data to generate a graphical dashboard that is accessible or rendered for display on an associated display device, and wherein: the graphical dashboard depicts a summary time-series chart for each user of their processed answer data relative to their baseline data on a category basis, each summary time-series chart depicting or representing deviations of the symptom data and/or medication data relative to their respective baseline data over a time period; and outputting the data report as a data file for rendering on a display or rendering the data report on an associated display.

In a configuration, the software application may further comprise: receiving a data request to view a data report associated with one or more users; and generating and rendering the data report on a display in response to the data request.

In an aspect, this disclosure broadly comprises a graphical user interface (GUI) and processor for displaying a graphical dashboard, the GUI being controlled by the processor to render the graphical dashboard on the GUI, the processor being configured to: retrieve or receive dashboard data representing the graphical dashboard into memory, the dashboard data comprising: answer data comprising symptom data and/or medication data representing user input to a set of presented queries relating to one or more health parameters from at least one of a symptoms category and a medication category, the answer data relating to multiple users of respective respiratory therapy devices; and baseline data associated with each respective user of the multiple users; process the dashboard data in accordance with rendering instructions; and render the graphical dashboard on the GUI based on the dashboard data and in accordance with the rendering instructions, the rendering instructions being configured to render a graphical dashboard that depicts a summary time-series chart for each user of their processed answer data relative to their baseline data on a category basis, each summary time-series chart depicting or representing deviations of the symptom data and/or medication data relative to their respective baseline data over a time period.

In an aspect, this disclosure broadly comprises a system comprising: a respiratory therapy device that is configured to deliver a flow of gases to a user for respiratory therapy comprising: a flow generator for generating the flow of gases, a humidifier for heating and humidifying the flow of gases, a controller that is operable to control the flow generator and humidifier, a user interface comprising a display to enable user interaction with the controller; a data server that is in data communication with the respiratory therapy device; and a display device that is in data communication with the data server, the controller of the respiratory therapy device being configured to: receive answer data based on user input to the user interface to an enquiry comprising a set of presented queries on the display relating to one or more health parameters from at least one of a symptoms category and a medication category, receive skipped data based on user input to the user interface to the set of presented queries, the skipped data representing any specific queries that are skipped by the user or the entire enquiry being skipped by the user and send the answer data and skipped data to the data server, the data server being configured to: receive and store the answer data and skipped data from the respiratory therapy device for a series of time intervals over a time period, process the answer data relative to baseline data associated with the user to determine deviations of the answer data relative to the baseline data for each health parameter, generate a data report that is accessible or rendered for display by the display device, the data report comprising one or more time-series charts depicting or representing deviations of the answer data relative to the baseline data over the series of time intervals, and wherein at least one of the time-series charts further comprises: skipped data graphical indicators identifying any queries within any of the time intervals that comprise queries that were skipped based on the skipped data, and no-data graphical indicators identifying any time intervals in which there is no answer data or skipped data, thereby indicating no user interaction with the respiratory therapy device during that time interval, and the generated data report being displayed or rendered on the display device.

In an aspect, this disclosure broadly comprises a system comprising: a respiratory therapy device that is configured to deliver a flow of gases to a user for respiratory therapy comprising: a flow generator for generating the flow of gases, a humidifier for heating and humidifying the flow of gases, a controller that is operable to control the flow generator and humidifier, a user interface comprising a display to enable user interaction with the controller; a data server that is in data communication with the respiratory therapy device; and a display device that is in data communication with the data server, the controller of the respiratory therapy device being configured to: receive answer data based on user input to the user interface to an enquiry comprising a set of presented queries on the display relating to one or more health parameters from at least one of a symptoms category and a medication category, receive skipped data based on user input to the user interface to the set of presented queries, the skipped data representing any specific queries that are skipped by the user or the entire enquiry being skipped by the user and send the answer data and skipped data to the data server, the data server being configured to: receive and store the answer data and skipped data from the respiratory therapy device for a series of time intervals over a time period, process the answer data relative to baseline data associated with the user to determine deviations of the answer data relative to the baseline data for each health parameter, generate a data report that is accessible or rendered for display by the display device, the data report comprising one or more time-series charts depicting or representing deviations of the answer data relative to the baseline data over the series of time intervals, and wherein at least one of the time-series charts further comprises: skipped data graphical indicators identifying any queries within any of the time intervals that comprise queries that were skipped based on the skipped data; and the generated data report being displayed or rendered on the display device.

In a configuration, at least one of the time-series charts may further comprise: no-data graphical indicators identifying any time intervals in which there is no answer data or skipped data, thereby indicating no user interaction with the respiratory therapy device during that time interval.

In a configuration, at least one of the time-series charts further comprises: a graphical indicator or indicators identifying any time intervals in which the user was in hospital or receiving hospital treatment.

In a configuration, at least one time-series chart comprises a colour-coded time-series plot of the answer data for the user for the one or more health parameters over a time period, and wherein each plotted answer of the answer data is selectively colour-coded for display according to a baseline colour spectrum or one or more deviation colour spectrums, the colour spectrum selected for each answer being a function of or based on any deviation of the answer data relative to the baseline data.

In a configuration, the plotted answers are grouped in rows or otherwise aligned with their associated health parameter when plotted against the time-axis of the colour-coded time-series plot.

In a configuration, the plotted answers are presented in defined category groups based on the category associated with the health parameter to which the plotted answer relates.

In a configuration, the answer data is plotted or populated into an array of colour-coded data cells arranged in rows according to the health parameters associated with the answers and columns according to the time interval of the captured answers along a time axis.

In a configuration, the colour-coded time-series plot further plots skipped data graphical indicators into data cells corresponding to skipped data.

In a configuration, skipped data is plotted into data cells according to skipped data graphical indicators that correspond to a presented key defining the skipped data graphical indicator.

In a configuration, the skipped data graphical indicators comprise any one of more of the following characteristics to differentiate it from answer data: colour-coding into a different colour from the baseline and deviation colour spectrums, patterns, shades, indicative text, or indicative symbols.

In a configuration, the colour-coded time-series plot further plots no-data graphical indicators into data cells for time-intervals in which no answer data or skipped data is received.

In a configuration, the no-data graphical indicators comprise any one or more of the following: colour-coding into a different colour from the baseline and deviation colour spectrums, patterns, shades, indicative text, indicative symbols, blank data cell, and blank column of data cells.

In a configuration, the answer data comprises symptom data and medication data relating to one or more health parameters from the respective symptoms category and medication category, and wherein at least one time-series chart comprises a time-series graph comprising plots representing deviations of both the symptom data and medication data relative to their respective baseline data for the series of time intervals over the time period.

In a configuration, the time-series graph comprises plots of the symptom data deviation and medication data deviation that are visually distinguishable from each other or visually represented as separate data sets within the same time-series graph.

In a configuration, the plots of the symptom data deviation and medication data deviation are represented by the same or different types or plots.

In a configuration, the plots representing the symptom data deviation and medication data deviation are selected from any one or more of the following types of plots: dot plot, line chart or line graph, bar chart, lollipop chart, stacked bar chart, and grouped bar chart.

In a configuration, the time-series graph comprises the symptom data deviation being plotted as a time-series bar chart and the medication data deviation being plotted as a time-series line chart, or vice versa, within the same time-series graph.

In a configuration, the time-series graph further plots skipped data graphical indicators indicative of time intervals comprising skipped data.

In a configuration, the time-series graph further plots no-data graphical indicators indicative of time-intervals in which no answer data or skipped data is received.

In a configuration, the display of the user interface comprises a touchscreen display providing a graphical user interface (GUI), and wherein the user input is provided via the touchscreen display and/or one or more input elements.

In a configuration, the display of the user interface presents a graphical user interface (GUI) and the user interface comprises one or more input elements that are operable by a user to provide user input to the GUI.

In a configuration, the presented queries on the display relate to one or more health parameters from a symptoms category or a medication category, or at least one health parameter from each category.

In an aspect, this disclosure broadly comprises a method of generating a data report for display, the method implemented by an electronic device comprising a processor having associated memory, and comprising: receiving or retrieving answer data into memory, the answer data representing user input to an enquiry comprising a set of presented queries relating to one or more health parameters from at least one of a symptoms category and a medication category for a user of a respiratory therapy device; receiving or retrieving skipped data into memory, the skipped data representing any specific queries that are skipped by the user or the entire enquiry being skipped by the user; receiving or retrieving baseline data into memory, the baseline data associated with the user; processing the answer data relative to the baseline data to determine deviations of the answer data relative to the baseline data for each health parameter for a series of time intervals over a time period; generating a data report that is accessible or rendered for display on a display device associated with or connected to the electronic device, and wherein: the data report comprises one or more time-series charts depicting or representing deviations of the answer data relative to the baseline data over the series of time intervals, and wherein at least one of the time-series charts further comprises: skipped data graphical indicators identifying any queries within any of the time intervals that comprise queries that were skipped based on the skipped data, and no-data graphical indicators identifying any time intervals in which there is no answer data or skipped data, thereby indicating no user interaction with the respiratory therapy device during that time interval; and displaying the generated data report on the display device.

In an aspect, this disclosure broadly comprises a method of generating a data report for display, the method implemented by an electronic device comprising a processor having associated memory, and comprising: receiving or retrieving answer data into memory, the answer data representing user input to an enquiry comprising a set of presented queries relating to one or more health parameters from at least one of a symptoms category and a medication category for a user of a respiratory therapy device; receiving or retrieving skipped data into memory, the skipped data representing any specific queries that are skipped by the user or the entire enquiry being skipped by the user; receiving or retrieving baseline data into memory, the baseline data associated with the user; processing the answer data relative to the baseline data to determine deviations of the answer data relative to the baseline data for each health parameter for a series of time intervals over a time period; generating a data report that is accessible or rendered for display on a display device associated with or connected to the electronic device, and wherein: the data report comprises one or more time-series charts depicting or representing deviations of the answer data relative to the baseline data over the series of time intervals, and wherein at least one of the time-series charts further comprises: skipped data graphical indicators identifying any queries within any of the time intervals that comprise queries that were skipped based on the skipped data; and displaying the generated data report on the display device.

In a configuration, at least one of the time-series charts further comprises: no-data graphical indicators identifying any time intervals in which there is no answer data or skipped data, thereby indicating no user interaction with the respiratory therapy device during that time interval.

In a configuration, at least one of the time-series charts further comprises: a graphical indicator or indicators identifying any time intervals in which the user was in hospital or receiving hospital treatment.

In a configuration, the electronic device is a computing device that is configured to receive the answer data from a data server and/or from a respiratory therapy device.

In a configuration, the electronic device is a data server that is configured to receive the answer data from a respiratory therapy device directly over a data communication network or indirectly via another electronic device over a data communication network.

In a configuration, the electronic device is a respiratory therapy device that is configured to deliver a flow of gases to a user for respiratory therapy comprising: a flow generator for generating the flow of gases, a humidifier for heating and humidifying the flow of gases, a controller that is operable to controller the flow generator and humidifier, a touchscreen providing a graphical user interface (GUI) to enable user interaction with the controller; and the controller of the respiratory therapy device being configured to receive answer data and skipped data based on user input to the touchscreen GUI to a set of presented queries relating to one or more health parameters.

In an aspect, this disclosure broadly comprises a software application for generating a data report, the application comprising a set of instructions for execution by a processor and associated memory of an electronic device to implement the steps of: receiving or retrieving answer data into memory, the answer data representing user input to an enquiry comprising a set of presented queries relating to one or more health parameters from at least one of a symptoms category and a medication category for a user of a respiratory therapy device; receiving or retrieving skipped data into memory, the skipped data representing any specific queries that are skipped by the user or the entire enquiry being skipped by the user; receiving or retrieving baseline data into memory, the baseline data associated with the user; processing the answer data relative to the baseline data to determine deviations of the answer data relative to the baseline data for each health parameter for a series of time intervals over a time period; generating a data report that is accessible or rendered for display on an associated display device, and wherein: the data report comprises one or more time-series charts depicting or representing deviations of the answer data relative to the baseline data over the series of time intervals, and wherein at least one of the time-series charts further comprises: skipped data graphical indicators identifying any queries within any of the time intervals that comprise queries that were skipped based on the skipped data, and no-data graphical indicators identifying any time intervals in which there is no answer data or skipped data, thereby indicating no user interaction with the respiratory therapy device during that time interval; and outputting the data report as a data file for rendering on a display or rendering the data report on an associated display.

In a configuration, the software application may further comprise: receiving a data request to view a data report associated with one or more users; and generating and rendering the data report on a display in response to the data request.

In an aspect, this disclosure broadly comprises a graphical user interface (GUI) and processor for displaying a data report, the GUI being controlled by the processor to render the data report on the GUI, the processor being configured to: receive or retrieve report data representing the data report into memory, the report data representing a series of time intervals over a time period and comprising: answer data representing user input to an enquiry comprising a set of presented queries relating to one or more health parameters from at least one of a symptoms category and a medication category for a user of a respiratory therapy device, skipped data representing any queries that are skipped by the user or the entire enquiry being skipped by the user, and baseline data associated with the user; process the report data in accordance with the rendering instructions; and render the data report on the GUI based on the report data and in accordance with rendering instructions, the rendering instructions being configured to render a data report that comprises one or more time-series charts depicting or representing deviations of the answer data relative to the baseline data over the series of time intervals, and wherein at least one of the time-series charts further comprises: skipped data graphical indicators identifying any queries within any of the time intervals that comprise queries that were skipped based on the skipped data, and no-data graphical indicators identifying any time intervals in which there is no answer data or skipped data, thereby indicating no user interaction with the respiratory therapy device during that time interval.

In an aspect, this disclosure broadly comprises a system comprising: a respiratory therapy device that is configured to deliver a flow of gases to a user for respiratory therapy comprising: a flow generator for generating the flow of gases, a humidifier for heating and humidifying the flow of gases, a controller that is operable to control the flow generator and humidifier, a user interface comprising a display to enable user interaction with the controller; a data server that is in data communication with the respiratory therapy device; and a display device that is in data communication with the data server, the controller of the respiratory therapy device being configured to receive answer data comprising symptom data and/or medication data based on user input to the user interface to a set of presented queries on the display relating to one or more health parameters from at least one of a symptoms category and a medication category, and send the answer data to the data server, the data server being configured to: receive the answer data from the respiratory therapy device for a series of time intervals over a time period, process the answer data relative to baseline data associated with the user to extract or generate symptom data deviation and/or medication data deviation in the form of one or more category deviation parameters for plotting that represent health parameters in each respective category for a user that have deviated from their respective baseline data for each time-interval, and generate a data report that is accessible or rendered for display by the display device, and wherein the data report comprises a time-series graph that plots the one or more category deviation parameters for the time intervals over the time period.

In a configuration, the data server is configured to: generate a symptom category deviation parameter or parameters representing or being a function of the health parameters that deviate from their baseline for each time-interval, and a medication category deviation parameter or parameters representing or being a function of the health parameters that deviate from their baseline for each time-interval, each deviation parameter being plotted in the time-series graph; or a worsening deviation parameter for each category representing or being a function of the total number of health parameters in the category that have a worsening deviation from their baseline for each time-interval, and an improvement deviation parameter for each category representing or being a function of the total number of health parameters in the category that have an improving deviation from their baseline for each time-interval, each deviation parameter being plotted in the time-series graph.

In a configuration, the data server is configured to generate the category deviation parameters for each time-interval as a function of or based on a colour-coding of the answer data, wherein the answer data is colour-coded according to either a baseline colour spectrum or a deviation colour spectrum depending on whether the answer deviates from baseline data for the user.

In a configuration, the data server is configured to generate the symptom category deviation parameter(s) and medication category deviation parameter(s) for each time-interval based on a data set representing a related colour-coded time-series plot of the answer data for the user for the one or more health parameters over a time period, wherein each plotted answer of the answer data is selectively colour-coded for display according to a baseline colour spectrum or one or more deviation colour spectrums, the colour spectrum selected for each answer being a function of or based on any deviation of the answer data relative to the baseline data.

In a configuration, the category deviation parameters for each time-interval are based on or are a function of the number of answers in each respective category that are colour coded into the deviation colour spectrum.

In a configuration, the time-series graph plots both a symptom category deviation parameter(s) and a medication category deviation parameter(s).

In a configuration, the plots of the symptom category deviation parameter(s) and medication category deviation parameter(s) are visually distinguishable from each other or visually represented as separate data sets within the same time-series graph.

In a configuration, the plots of the symptom category deviation parameter(s) and medication category deviation parameter(s) are represented by the same or different types of plots.

In a configuration, the plots of the symptom category deviation parameter(s) and medication category deviation parameter(s) are represented by independent plots within the same time-series graph or as combined plots within the same time-series graph.

In a configuration, the plots of the symptom category deviation parameter(s) and medication category deviation parameter(s) are selected from any one or more of the following types of plots: dot plot, line chart or line graph, bar chart, lollipop chart, stacked bar chart, and grouped bar chart.

In a configuration, the time-series graph comprises the symptom category deviation parameter(s) being plotted as a bar chart and the medication category deviation parameter(s) being plotted as a line chart, or vice versa.

In a configuration, the bar chart and line chart representing the symptom category deviation parameter(s) and medication category deviation parameter(s) are plotted relative to the same deviation axis such that they overlay each other visually.

In a configuration, the bar chart and line chart representing the symptom category deviation parameter(s) and medication category deviation parameter(s) are visually distinguished from each other by colour, pattern, or shade.

In a configuration, the data server is configured to process the answer data relative to the baseline data such that the generated one or more category deviation parameter(s) can represent both worsening and improvement in the deviation parameter.

In a configuration, the data server is further configured to receive prescription data indicative of time-intervals in which a change or update in prescription for the user has occurred, and to plot a graphical indication or representation in the time-series graph indicating the prescription change in the relevant time-intervals.

In an aspect, this disclosure broadly comprises a method of generating a data report for display, the method implemented by an electronic device comprising a processor having associated memory, and comprising: receiving or retrieving answer data into memory, the answer data comprising symptom data and/or medication data based on user input to a set of presented queries relating to one or more health parameters from at least one of a symptoms category and a medication category for a user of a respiratory therapy device; receiving or retrieving baseline data into memory, the baseline data associated with the user; processing the answer data relative to the baseline data for a series of time-intervals of a time period to extract or generate symptom data deviation and/or medication data deviation in the form of one or more category deviation parameters for plotting that represent health parameters in each respective category for a user that have deviated from their respective baseline data for each time-interval; and generating a data report that is accessible or rendered for display on a display device associated with or connected to the electronic device, and wherein the data report comprises a time-series graph that plots the one or more category deviation parameters for the time intervals over the time period; and displaying the generated data report on the display device.

In a configuration, the electronic device is a computing device that is configured to receive the answer data from a data server and/or from a respiratory therapy device.

In a configuration, the electronic device is a data server that is configured to receive the answer data from a respiratory therapy device directly over a data communication network or indirectly via another electronic device over a data communication network.

In a configuration, the electronic device is a respiratory therapy device that is configured to deliver a flow of gases to a user for respiratory therapy comprising: a flow generator for generating the flow of gases, a humidifier for heating and humidifying the flow of gases, a controller that is operable to controller the flow generator and humidifier, a touchscreen providing a graphical user interface (GUI) to enable user interaction with the controller; and the controller of the respiratory therapy device being configured to receive answer data and skipped data based on user input to the touchscreen GUI to a set of presented queries relating to one or more health parameters.

In an aspect, this disclosure broadly comprises a software application for generating a data report, the application comprising a set of instructions for execution by a processor and associated memory of an electronic device to implement the steps of: receiving or retrieving answer data into memory, the answer data comprising symptom data and/or medication data based on user input to a set of presented queries relating to one or more health parameters from at least one of a symptoms category and a medication category for a user of a respiratory therapy device; receiving or retrieving baseline data into memory, the baseline data associated with the user; processing the answer data relative to the baseline data for a series of time-intervals of a time period to extract or generate symptom data deviation and/or medication data deviation in the form of one or more category deviation parameters for plotting that represent health parameters in each respective category for a user that have deviated from their respective baseline data for each time-interval; generating a data report that is accessible or rendered for display on an associated display device, and wherein the data report comprises a time-series graph that plots the one or more category deviation parameters for the time intervals over the time period; and outputting the data report as a data file for rendering on a display or rendering the data report on an associated display.

In a configuration, the software application may further comprise: receiving a data request to view a data report associated with one or more users; and generating and rendering the data report on a display in response to the data request.

In an aspect, this disclosure broadly comprises a graphical user interface (GUI) and processor for displaying a data report, the GUI being controlled by the processor to render the data report on the GUI, the processor being configured to: retrieve or receive report data representing the data report into memory, the report data representing a series of time intervals over a time period and comprising: answer data comprising symptom data and/or medication data representing user input to a set of presented queries relating to one or more health parameters from at least one of a symptoms category and a medication category for a user of a respiratory therapy device, and baseline data associated with the user; process the report data in accordance with rendering instructions; and rendering the data report on the GUI based on the report data and in accordance with the rendering instructions, the rendering instructions being configured to render a data report that comprises a time-series graph that plots one or more category deviation parameters that represent health parameters in each respective category for a user that have deviated from their respective baseline data for each time-interval in the time-period.

In an aspect, this disclosure broadly comprises a system comprising: a respiratory therapy device that is configured to deliver a flow of gases to a user for respiratory therapy comprising: a flow generator for generating the flow of gases, a humidifier for heating and humidifying the flow of gases, a controller that is operable to control the flow generator and humidifier, a user interface comprising a display to enable user interaction with the controller; a data server that is in data communication with the respiratory therapy device; the controller of the respiratory therapy device being configured to receive enquiry data based on user input to the user interface to a set of presented queries of a personal health enquiry on the display relating to one or more health parameters from at least one of a symptoms category and a medication category, and send the enquiry data to the data server, the data server being configured to: receive the enquiry data from the respiratory therapy device; process the enquiry data relative to one or more stored notification profiles linked or assigned to the user, the one or more notification profiles each comprising data defining trigger criteria; and trigger one or more notifications according to the notification profiles to be generated on the respiratory therapy device, or an electronic device in data communication with the respiratory therapy device or data server, if the respective trigger criteria for the one or more notification profiles is satisfied by the processed enquiry data.

In an aspect, this disclosure broadly comprises a system for processing enquiry data comprising the answers of a user to a personal health enquiry comprising a set of queries relating to one or more health parameters from at least one of a symptoms category and a medication category, the system comprising: a processor and memory, the processor being configured to: receive or retrieve the enquiry data from a respiratory therapy device or electronic device operated by the user; process the enquiry data relative to one or more stored notification profiles linked or assigned to the user, the one or more notification profiles each comprising data defining trigger criteria; and trigger one or more notifications according to the notification profiles to be generated on an electronic device in data communication with the processor if the respective trigger criteria for the one or more notification profiles is satisfied by the processed enquiry data.

In a configuration, each of the notification profiles further comprises data defining a notification message that defines a message, content, or information to be conveyed in the notification.

In a configuration, the notification message and/or trigger criteria of the notification profiles is configurable or adjustable.

In a configuration, the system comprises a user interface that is operable to create, configure or adjust the one or more notification profiles.

In a configuration, the user interface is configured to enable a user to configure or adjust the trigger criteria for the one or more notification profiles.

In a configuration, the one or more notification profiles assigned to the user comprise one or more default notification profiles that are assigned to the user based on a patient profile defining the user.

In a configuration, the system links or assigns the patient profile of a user to one or more patient groups if their patient profile satisfies the respective patient group criteria, and the one or more notification profiles assigned to the user comprise one or more patient group notification profiles.

In a configuration, the patient group notification profiles are notification profiles that apply to all users that are members of the associated patient group based on their patient profile.

In a configuration, the system comprises a user interface that is operable to create or configure patient groups and their associated patient group criteria.

In a configuration, the system comprises a user interface that is operable to create, configure or adjust the one or more patient group notification profiles for the patient groups defined in the system.

In a configuration, the trigger criteria of one or more of the notification profiles is configurable via an operable user interface of the system.

In a configuration, the trigger criteria of one or more of the notification profiles comprises criteria that is at least partly based on or a function of the enquiry data and associated stored baseline data of the user, and one or more conditions.

In a configuration, the trigger criteria of one or more of the notification profiles comprises criteria that is at least partly based on or a function of deviation data representing the deviation of the enquiry data from the baseline data of the user, and one or more conditions.

In a configuration, the trigger criteria of one or more of the notification profiles comprises criteria that is at least partly based on or a function of enquiry data and associated stored baseline data relating to one or more specified health parameters associated with the personal health enquiry, and one or more conditions.

In a configuration, the trigger criteria of one or more of the notification profiles comprises criteria that is at least partly based on or a function of deviation data representing the deviation of the enquiry data from the baseline data of the user for one or more specified health parameters associated with the personal health enquiry, and one or more conditions.

In a configuration, the conditions of the trigger criteria comprise comparing data to a threshold or thresholds and/or time period conditions.

In a configuration, the triggered notifications comprise any one or more of the following: visual notifications, audible notifications, and/or tactile notifications.

In an aspect, this disclosure broadly comprises a data server configured to process enquiry data comprising the answers of a user to a personal health enquiry comprising a set of queries relating to one or more health parameters of the user, the data server comprising a processor that is configured to: receive or retrieve the enquiry data from an electronic device, database or a respiratory therapy device; process the enquiry data relative to one or more stored notification profiles linked or assigned to the user, the one or more notification profiles each comprising data defining trigger criteria; and trigger one or more notifications according to the notification profiles to be generated on an electronic device in data communication with the data server if the respective trigger criteria for the one or more notification profiles is satisfied by the processed enquiry data.

In an aspect, this disclosure broadly comprises a system comprising: a data server comprising an associated processor or processors and memory; and a display device that is in data communication with the data server, the data server being configured to: receive answer data representing answers from a user to a set of presented queries relating to one or more health parameters from at least one of a symptoms category and a medication category; and process the answer data relative to baseline data associated with the user to generate a data report that is accessible or rendered for display by the display device, and wherein: the data report comprises a colour-coded time-series plot of the answer data for a user for the one or more health parameters over a time period, and wherein each plotted answer of the answer data is selectively colour-coded for display according to a baseline colour spectrum or one or more deviation colour spectrums, the colour spectrum selected for each answer being a function of or based on any deviation of the answer data relative to the baseline data, and the generated data report being displayed or rendered on the display device.

In an aspect, this disclosure broadly comprises a non-transitory computer-readable medium having stored thereon computer executable instructions that, when executed on a processing device or devices, cause the processing device or devices to perform any method of any one or more aspects above.

In an aspect, this disclosure broadly comprises a set of application program interfaces or an application program interface (API) embodied on a computer-readable medium for execution on a processing device in conjunction with an application program that perform any method of any one or more aspects above.

Any aspect of this disclosure above may further comprise any one or more aspects or features mentioned in respect of any one or more of the other aspects, or wherein any aspect of the storage, transmission, processing, display, and/or rendering of any data (e.g., enquiry data, answer data, symptom data, medication data, category deviation parameters, baseline data, and/or deviation data), data reports, graphical dashboard, summary time-series chart, time-series charts (e.g., colour-coded time-series plot, and/or time-series graph), and/or any aspect of the related system architecture may have any one or more of the features mentioned in respect of any other aspect described above, in any combination.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 shows a schematic diagram of a breathing assistance apparatus in accordance with an embodiment;

FIG. 2 is a front/right side overhead perspective view of a breathing assistance apparatus with a humidifier liquid chamber positioned in the recess of the breathing assistance apparatus base unit in accordance with an embodiment;

FIG. 3 is a front/left side of the breathing assistance apparatus of FIG. 2 with the humidifier liquid chamber removed from the recess of the breathing assistance apparatus in accordance with an embodiment;

FIG. 4 is a front/right side overhead perspective view of the breathing assistance apparatus base unit with the humidifier liquid chamber and heater plate not shown in accordance with an embodiment;

FIG. 4A is a front/right side bottom perspective view of the breathing assistance apparatus with the humidifier liquid chamber not shown in accordance with an embodiment;

FIG. 4B is a rear view of the breathing assistance apparatus base unit with the battery module in place in accordance with an embodiment;

FIG. 5 is a schematic of a breathing assistance apparatus in accordance with an embodiment;

FIGS. 6, 6A and 6B are flowcharts for the controller operation in accordance with embodiments;

FIG. 7 is a flowchart of a process for conditionally presenting a personal health enquiry to a patient of a breathing assistance apparatus in accordance with an embodiment;

FIG. 8 is a flowchart of a process for determining deviations to answers of a personal health enquiry based on preset baselines;

FIGS. 9-13, 13A, 14, 14A, and 15-18 show a user interface presenting a personal health enquiry on a breathing assistance apparatus in accordance with an embodiment;

FIG. 19 is a system architecture diagram showing a data system with a breathing assistance apparatus, data server, and display device in accordance with an embodiment;

FIG. 20 is a system architecture diagram showing a data system of FIG. 19 with the data server serving multiple breathing assistance apparatuses and multiple display devices in accordance with an embodiment;

FIG. 21 is a flowchart of a data processing flow for the personal health enquiry data in accordance with an embodiment;

FIG. 22 is a representation of a digital patient data report comprising processed personal health enquiry data that is rendered on a display device in accordance with an embodiment;

FIG. 23 is a representation of a digital colour-coded time-series plot or heatmap of processed personal health enquiry data that is rendered on a display device in accordance with an embodiment;

FIG. 24 is a close-up view of a key of the digital colour-coded time-series plot or heatmap of FIG. 23 in accordance with an embodiment;

FIGS. 25A-25C show representations of various embodiments of a time-series graph of category deviation parameters generated from the personal health enquiry data in accordance with an embodiment;

FIG. 26 shows a representation of a digital graphical dashboard depicting multiple summary time-series charts relating to multiple patients in accordance with an embodiment;

FIG. 27 is a flowchart of a data processing flow for modifying the queries in the personal health enquiry based on patient physiological parameter data in accordance with an embodiment; and

FIG. 28 is a flowchart of a data processing flow for triggering additional follow-on queries in the personal health enquiry based on or as a function of answers to prior queries in accordance with an embodiment.

### DETAILED DESCRIPTION

### 1. Overview

Patients who suffer from COPD, or bronchiectasis, or other respiratory distress are often treated with various therapies such as for example bilevel pressure therapy and/or nasal high flow therapy in a hospital. Nasal high flow therapy is one commonly used therapy for treating COPD or bronchiectasis patients in a hospital. When these patients are discharged from the hospital, his or her doctor (i.e. physician) may wish for them to continue with regular high flow therapy at home, including a retirement village or a hospice, or a location outside of hospital. In such cases, his or her doctor will prescribe in-home high flow therapy and his or her healthcare provider will provide them with a nasal high flow device suited to in-home use. Alternatively, the healthcare provider may prescribe a therapy for use at home, for example nasal high flow at home. The patient may contact an equipment provider who may provide a medical device to the patient based on the prescription.

The prescription may be or may comprise a flow rate that is set by a clinician for a specific patient. The prescription may be or may comprise a prescribed therapy time such as, but not limited to, therapy time per day or per week, and/or number of specific therapy sessions per day or per week, for example.

The prescription may be a combination of flow rate and/or O2 concentration (i.e. O2%) and/or humidity level (e.g. dew point or RH or absolute humidity). The prescription may be loaded and stored in the memory of the apparatus.

Prescription is preferably determined by a clinician prior to discharge from the hospital or during regular checkups. The prescription may be updated by the clinician.

Healthcare providers often feel that it is prudent (for example financially and/or clinically) to cover the costs of providing discharged COPD patients with in-home nasal high flow devices, as in-home nasal high flow treatment may provide respiratory support to a patient therefore not requiring the patient to remain in hospital and/or reduce the likelihood of exacerbations and, therefore, expensive hospital readmissions.

Unfortunately, some patients that are treated with in-home therapy e.g. nasal-high flow therapy may still experience a COPD exacerbation (i.e. a flare up or worsening of COPD symptoms), or a deterioration of other health conditions that can lead to hospitalization. As such, an ability to predict or detect when a patient's medical state is deteriorating towards a state or point in which hospitalization is required (for example by monitoring a patient's condition) would be beneficial for both patients and healthcare providers: doctors would be able to step in and treat the patients before they suffer a significant exacerbation (for example a worsening of symptoms) of COPD that would require hospitalization, or, more generally, before they reach a condition that requires expensive hospitalization.

The patient's condition may be monitored over a period of time and then based on the monitoring a prediction may be made as to whether a patient's medical state is going to deteriorate to the point of hospitalization (for example before an exacerbation).

One way to gather the necessary information, for predicting a medical state which requires hospitalization, is to have patients regularly answer queries about his or her current state of health. However, this is problematic because patients, who are receiving in-home treatment, may be too unwell or may be quite old or weak to regularly fill out questionnaires on paper or with an additional device (e.g., mobile phone, tablet, desktop, laptop). Furthermore, even if patients are physically well enough, they may self-diagnose or this task may become so tedious that they disengage and stop doing it-especially if the enquiry is not user friendly (e.g., a paper form or a poorly laid out digital enquiry). Using paper or an additional user device (e.g. a mobile phone, tablet, desktop, laptop) to complete the questionnaire can be tedious for a patient because the patient has to use another device and hence patient's often don't complete questionnaires provided on paper or other devices.

The present disclosure provides a system for digital data capture of personal health enquiry data from users or patients of breathing assistance apparatus, and for remote storage and processing of the raw data by a remote or cloud-based data server system. The data server is accessible by healthcare providers to access digital patient data reports compiled based on the processed data, including coded time-series charts for graphical rendering. The time-series charts can be generated dynamically or in real-time upon request, and the format and content is configurable based on user preference settings. The graphical time-series charts may be interactive. A graphical dashboard is generated in some configurations that depicts multiple summary graphical data extracts or time-series charts for multiple patients for efficient review and monitoring by a healthcare provider that is responsible for multiple patients. The graphical charts may process the raw data to present or identify deviations (e.g. worsening health parameters or health conditions, and/or alternatively improvements in health parameters or health conditions) for easy review by the healthcare provider. The coding of the data for the graphical representations may provide for efficient transmission and rendering for display, relative to the raw data, in some configurations.

The present disclosure provides systems and methods to enable healthcare providers to remotely capture health condition information from multiple patients while they are at their homes and using a breathing assistance apparatus for respiratory therapy, and to review the captured data in data reports and/or receive default and/or custom notifications based on the captured data. This remote digital data capture, processing and reporting, may enable a healthcare provider to more effectively and efficiently review patients under their care, triage the patients in terms of priority, revise or adjust their prescribed therapy or otherwise intervene or vary treatment, and/or generally monitor the patient's condition, risk of exacerbation, and/of effectiveness of their prescribed therapy.

In the following, an example of a breathing assistance apparatus for capturing a personal health enquiry from a patient or user of the apparatus will be described. The personal health enquiry may be a digital questionnaire comprising a series or set of queries related to one or more health parameters (e.g. symptoms and medications used), including objective and/or subjective queries. Following this, the process of processing the personal health enquiry data, parameter generation and data coding, and the rendering of the data reports, time-series charts, and/or graphical dashboard will be explained by way of example.

### 2. Example Breathing Assistance Apparatus

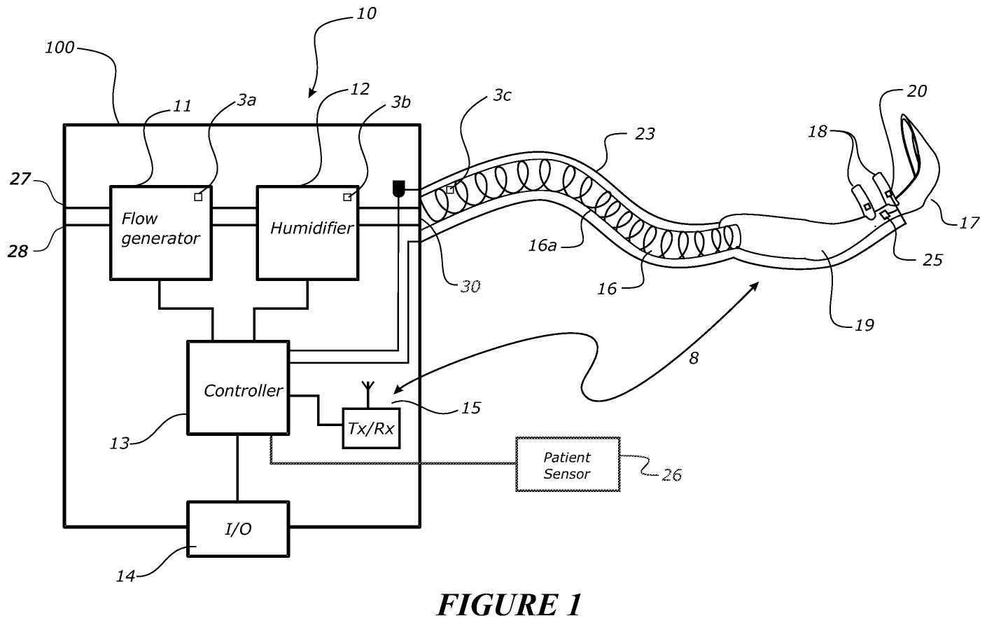

A breathing assistance apparatus as a high flow apparatus for delivering a flow of gas (which may contain one or more gases) to a patient for high flow therapy is shown in FIG. 1. The breathing assistance apparatus may also be referred to as a ‘respiratory therapy device’ herein.

Alternatively or additionally, the apparatus could, for example, be a CPAP apparatus and/or a Bi-level device and/or Non-invasive Ventilation (NIV) apparatus (or any combination thereof). The apparatus could, for example, provide or selectively operate in modes that provide CPAP therapy and/or Bi-level pressure therapy and/or NIV therapy (or any combination thereof).

Different patient interfaces may be provided depending on the therapy type. For example, a non-sealing interface may be provided for high flow therapy, and a sealing interface may be provided for CPAP therapy.

An exemplary apparatus for delivering CPAP therapy is described in PCT Patent Application Publication WO2011/056080, filed 8 Oct. 2010. The contents of that specification are incorporated herein in its entirety by way of reference.

A breathing assistance apparatus comprises a gases supply and optionally gases humidification apparatus. The breathing assistance apparatus is operable to provide respiratory assistance to patients or users who require a supply of gas (humidified or otherwise) at positive pressure for the treatment of diseases such as Obstructive Sleep Apnea (OSA), snoring, or Chronic Obstructive Pulmonary Disease (COPD) and the like. A breathing assistance apparatus would typically include a humidifier chamber as a humidifier liquid chamber, so as to form a combined assisted breathing unit and humidifier.

Breathing assistance apparatuses, when used with a humidifier, typically have a structure where gases are delivered from an assisted breathing unit or blower unit to a liquid chamber downstream from the blower. As the gases pass through the liquid chamber, they become saturated with liquid vapour (e.g. water vapour). A flexible tubular gases conduit delivers the gases to a user or patient downstream from the humidifier chamber.

A high flow apparatus may be used to deliver a high gas flow or high flow therapy to a patient to assist with breathing and/or treat breathing disorders including chronic obstructive pulmonary disease (COPD) or respiratory distress syndrome or dyspnea, or bronchiectasis. A high flow apparatus includes a gases supply and typically includes a humidification apparatus. A high flow apparatus may provide respiratory support to a patient.

A CPAP apparatus may be used to deliver a continuous positive airway pressure to patient, or CPAP therapy to a patient (as described in more detail below).

A Bi-level apparatus may be used to deliver a bi-level pressure to the patient, or Bi-level therapy to a patient (as described in more detail below).

The breathing assistance apparatuses typically have one or more accessories such as a breathing conduit and a patient interface such as a cannula or mask for delivering gases to a patient. The conduit enables gases to be delivered from the housing of the breathing assistance apparatus to the patient. For example, the apparatus may be placed on a floor or other support surface, and the patient may be in a bed.

The breathing assistance apparatus may have a recess for receipt of a humidifier liquid chamber. The liquid chamber will receive liquid from, for example, a flexible liquid bag that delivers liquid to a humidifier liquid chamber via one more tubes. Alternatively, the liquid chamber can be removed and refilled as required. The recess will contain a heater plate to heat the liquid chamber, to humidify gases passing through the liquid chamber. The humidified gases are then delivered to the patient.

FIG. 1 shows an example breathing assistance apparatus 10. The breathing assistance apparatus 10 is configured to provide high flow therapy and function as a high flow apparatus.

The breathing assistance apparatus 10 is alternatively or additionally configured to provide pressure therapy and can function as a pressure therapy apparatus.

The breathing assistance apparatus 10 may be configured to provide high flow of gases when in a high flow mode. The breathing assistance apparatus 10 may be configured to operate in a pressure therapy mode, where the breathing assistance apparatus 10 provides a pressure therapy. The pressure therapy may be a positive bi-level pressure therapy or a constant positive pressure therapy. For example, in bi-level pressure therapy the apparatus 10 is configured to control the gases flow such that the patient receives an inspiratory pressure and an expiratory pressure, wherein both pressures are positive pressures. In constant pressure therapy the apparatus 10 is configured to deliver gases at a constant pressure that may be user set. The level or pressure may be set by a healthcare provider (e.g. a physician). An unsealed patient interface is used to deliver high flow therapy e.g. a nasal cannula. Conversely a sealed patient interface is used to deliver a pressure therapy (e.g. a nasal mask or a full face mask or nasal pillows).

In general terms, the apparatus 10 comprises a main housing 100 that contains a flow generator 11, a humidifier 12, a controller 13, and a user I/O interface 14.

In some embodiments, the user I/O interface comprises a display and one or more user input device or elements that are operable by a user to provide user input. The user input device or elements may comprise one or more button(s), switches, or dials (e.g. mechanical, touch, capacitive or similar) and/or a touch-screen, for example. In embodiments in which the user interface comprises a touch-screen display, user input may be provided via touch interaction with one or more graphical input elements presented on a graphical user interface (GUI) on the display.

In some embodiments the user I/O interface may be part of an ancillary device. The ancillary device may be for example a phone, tablet, or computer. The ancillary device may be configured to communicate directly with the apparatus, or it may be configured to communicate with the apparatus via one or more servers or a data network.

In some embodiments, the queries of the enquiry may be presented on the user I/O interface of the ancillary device, and the user may answer the queries on the ancillary device.

In some embodiments, the answers to the queries may be stored on the ancillary device and transmitted to the apparatus (for example at the end of the enquiry, or as each query is answered, or at a later time). In some embodiment the apparatus may then upload the answers to the queries (along with other information, for example patient parameters) to the patient and device management platform.

The ancillary device may upload the answers to the queries (optionally along with other information, for example patient parameters, from the apparatus) to the patient and device management platform.

In some embodiments the touchscreen may be provided as, or as part of, the user I/O interface 14. As discussed above, in some configurations the touchscreen may be the primary or sole user input device, with the user interacting with the graphical user interface via touch interactivity (e.g. touches, pushes, flicks, swipes, single-finger or multi-finger gestures or interactions, e.g. pinch to zoom etc) with the touchscreen. In other configurations, one or more additional user input elements or devices may be provided in combination with the touchscreen, such as buttons, dials, switches or similar (whether mechanical or touch-sensitive). Such additional user input elements or devices may typically be co-located with the touchscreen such as provided adjacent or near the touch-screen or about its periphery or may be displaced away from the touchscreen on another accessible part or surface of the housing of the breathing assistance apparatus. The one or more additional user input elements or devices may be used in combination with touch interactivity with the touchscreen or as an alternative to touch interactivity, to interact with the displayed GUI on the touchscreen, e.g. to navigate menus, functions, enter information or responses, configure settings and user preferences, and/or to interact with any other control or apparatus function.

In some embodiments the controller may be comprised of a plurality of controllers to control different components of the apparatus 10.

The plurality of controllers may comprise one or more of: a controller for the user I/O interface, a controller to control the flow generator and/or the humidifier, a controller to receive sensor inputs.

In some embodiments the controller to control the flow generator and humidifier is configured to receive input from the other controllers (for example the controller to receive sensor inputs, and user controller for the user I/O interface).

The plurality of controllers may be configured to communicate with each other (for example via a bus) and/or communicate to a master controller.

The flow generator 11 may comprise a motor/impeller arrangement e.g. a blower or pump or may comprise a compressor or other suitable component to create a flow of gases.

The controller 13 is configured or programmed to control the components of the apparatus, including: operating the flow generator 11 to create a flow of gas (gas flow) for delivery to a patient, operating the humidifier 12 to humidify and/or heat the generated gas flow, receive user input from the user interface 14 for reconfiguration and/or user-defined operation of the apparatus 10, and output information (for example on the display) to the user via the user interface 14.

In some embodiments the controller is configured to receive a start-up input/request via the user interface. The startup input/request activates the apparatus to initiate the enquiry to the user.

The user could be a patient, healthcare provider, or anyone else interested in using the apparatus. In one example where the apparatus is used in an out of hospital setting e.g. in the home or in a hospice or retirement village or other non-hospital setting, the user of the apparatus 10 is the patient. The patient will use the device to receive high flow therapy or pressure therapy according to a prescription from a healthcare provider (e.g. a physician).

It will be appreciated that in the context of answering the queries (as described elsewhere) the user will preferably be the patient as the queries relate to the health parameters of the patient.

A patient breathing conduit 16 is connected to a gas flow output or patient outlet port 30 (i.e. outlet port) in the housing 100 of the breathing assistance apparatus 10, and is connected to a patient interface 17 such as a nasal cannula with a manifold 19 and nasal prongs 18. In some embodiments, the nasal cannula may be sealed or un-sealed (for example when used to provide high flow therapy).

Additionally, or alternatively, the patient breathing conduit 16 could be connected to a face mask (for example a sealed mask when pressure therapy, such as CPAP or Bi-level pressure therapy, is provided).

Additionally, or alternatively, the patient breathing conduit could be connected to a nasal pillows mask, and/or a nasal mask, and/or a tracheostomy interface, or any other suitable type of patient interface.

The gas flow, which may be humidified, that is generated by the breathing assistance apparatus 10 is delivered to the patient via the patient breathing conduit 16 (and optionally via a humidifier) through the patient interface 17. The patient breathing conduit 16 can have a heater wire 16a to heat gas flow passing through to the patient. The heater wire 16a is controlled by the controller 13. Alternatively, the breathing assistance apparatus comprises a separate heater wire controller that controls an output (for example power or current or voltage) to the heater wire.

The patient breathing conduit 16 and/or patient interface 17 can be considered part of the breathing assistance apparatus 10, or alternatively peripheral to it. The breathing assistance apparatus 10, breathing conduit 16, and patient interface 17 may together form a breathing assistance system, for example a flow therapy system for providing high flow respiratory support i.e. high flow respiratory therapy to a patient as illustrated in FIG. 1.

In some embodiments, the controller 13 controls the flow generator 11 to generate a gas flow of the desired flow rate.

In some embodiments, the controller controls one or more valves to control the mix of air and oxygen or other alternative gas.

In some embodiments, the controller controls the humidifier 12 to humidify the gas flow and/or heat the gas flow to a desired level and/or in accordance with the temperature and/or humidity level settings of the therapy being provided. For example, the controller may control the power applied to the heater plate of the humidifier and/or heater element in the patient breathing conduit to achieve the desired temperature and/or humidity settings for the flow of gases to the patient, with the control being based at least partly on one or more temperature sensors and/or humidity sensors that are arranged to sense or measure the temperature and/or humidity of the flow of gases generated by the apparatus.

The gas flow is directed out through the patient breathing conduit 16 and patient interface 17 to the patient. As discussed above, the controller 13 can also control a heating element in the humidifier 12 and/or the heating element 16a in the patient breathing conduit 16 to humidify and/or heat the gas to a desired temperature that achieves a desired level of therapy and/or comfort for the patient.

The controller 13 can be programmed with, or can determine, a suitable target temperature of the gas flow.

The controller 13 controls the flow generator to generate a gases flow at a desired flow rate based on feedback from a flow sensor when in a high flow therapy mode. Alternatively, the controller 13 is configured to control the flow generator to generate a gases flow at a desired pressure based on feedback from a pressure sensor (e.g. a differential pressure sensor) when in pressure therapy mode.

Operation sensors 3a, 3b, 3c, 20, and 25, such as flow, temperature, humidity, and/or pressure sensors, can be placed in various locations in the breathing assistance apparatus 10 and/or the patient breathing conduit 16 and/or patient interface 17.

In some embodiments, at least one of the operation sensors 3a, 3b, 3c, 20, and 25 is provided within a sensor module. The sensor module is be located in the gases flow path. The sensor module may be located in the gases flow path between the flow generator (for example the blower) and the humidifier.

At least one of the operation sensors 3a, 3b, 3c, 20, and 25 may be provided within the gases flow path to sense a parameter of the gases flow.

In some embodiments, the apparatus may measure or determine one or more patient parameters. The patient parameters may be for example one or more of: respiratory rate, oxygen saturation of the patient, and/or other physiological parameters or indexes (e.g., respiratory rate to oxygenation (ROX) index). The patient parameters may be or relate to one or more physiological parameters of the patient. The patient parameter or parameters may be measured by the patient sensor (as described below) or determined based on sensors from the apparatus.

In some embodiments, the respiratory rate may be determined based on a flow signal from a flow sensor. The respiratory rate may be for example determined as disclosed in PCT Patent Application Publication WO2019/102384, filed 22 Nov. 2018. The contents of that specification are incorporated herein by way of reference.

In some embodiments, the controller of the apparatus may be configured to receive and/or determine respiratory rate data for the patient. In one configuration, the respiratory rate may be determined by the controller, for example as a function of two or more of the following sensed or measured gases flow or apparatus parameters: pressure of the gases flow, flow rate of the gases flow, and/or motor speed of the flow generator. In another configuration, the controller may receive the respiratory rate data for the patient from an external device or sensor such as, but not limited to, a pulse oximeter or wearable sensor.

Also included is a patient sensor 26. The patient sensor 26 may be a sensor that is mounted on the patient or associated with the patient to measure the patient parameter. In one example the patient sensor 26 is pulse oximeter that measures the oxygen saturation of the patient i.e. SpO2 value. Output from the sensors can be received by the controller 13, to assist it to operate the breathing assistance apparatus 10 in a manner that provides optimal therapy.

In some configurations, providing optimal therapy includes meeting, or exceeding, a patient's inspiratory flow. The apparatus 10 may have a transmitter and/or receiver 15 (for example as part of the network interface described in more detail below) to enable the controller 13 to receive signals 8 from the sensors and/or to control the various components of the breathing assistance apparatus 10, including but not limited to the flow generator 11, humidifier 12, and heater wire 16a, or accessories or peripherals associated with the breathing assistance apparatus 10.

Additionally, or alternatively, the transmitter and/or receiver 15 may deliver data to an external or remote service or platform or data server. For example, a remote patient and device management platform. The patient and device management platform may be any one or a combination of a remote device, data server, an application, a cloud service or platform (for example distributed computer system resources) or any other suitable hardware and software platform. In one embodiment the transmitter and/or receiver 15 may enable remote control of the apparatus 10.

In one embodiment, the transmitter and/or receiver 15 may comprises a GSM module or chip, or any other suitable data, mobile, or cellular communication module. The transmitter and/or receiver 15 of the apparatus may comprise one or more communication modules. In one example configuration, the apparatus may comprise a GSM module, and additionally one or more other additional communication modules such as, but not limited to, a Bluetooth module, Wifi module, ZigBee module, Z-wave module, NFC module, RFID module, satellite module, LAN or WLAN module, infrared module, radio module, modem module, or any other communication module. In other configurations, the apparatus may comprise any combination of one or more of the aforementioned communication modules, with or without a GSM module.

The breathing assistance apparatus 10 may be any suitable type of apparatus, but in some configurations may deliver a high gas flow or high flow therapy (of e.g. air, oxygen, other gas mixture, or some combination thereof) to a patient to assist with breathing and/or treat breathing disorders. In some configurations, the gas is or comprises oxygen. In some configurations, the gas comprises a blend of oxygen and ambient air. High flow therapy as discussed herein is intended to be given its typical ordinary meaning as understood by a person of skill in the art which generally refers to a respiratory assistance system delivering a targeted flow of respiratory gases (preferably humidified gas) via an intentionally unsealed patient interface with flow rates generally intended to meet or exceed inspiratory flow of a patient. Typical patient interfaces include, but are not limited to, a nasal or tracheal patient interface. Typical flow rates for adults often range from, but are not limited to, about fifteen liters per minute (LPM) to about seventy liters per minute or greater. Typical flow rates for pediatric patients (such as neonates, infants and children) often range from, but are not limited to, about one liter per minute per kilogram of patient weight to about three liters per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (THF), among other common names.

For example, in some configurations, for an adult patient ‘high flow therapy’ may refer to the delivery of gases to a patient at a flow rate of greater than or equal to about 10 liters per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may, in some configurations, deliver gases to the patient at a flow rate of between about 1 LPM and about 100 LPM, or at a flow rate in any of the sub-ranges outlined above. Gases delivered may comprise a percentage of oxygen. In some configurations, the percentage of oxygen in the gases delivered may be between about 20% and about 100%, or between about 30% and about 100%, or between about 40% and about 100%, or between about 50% and about 100%, or between about 60% and about 100%, or between about 70% and about 100%, or between about 80% and about 100%, or between about 90% and about 100%, or about 100%, or 100%.

High flow therapy has been found effective in meeting or exceeding the patient's inspiratory flow, increasing oxygenation of the patient and/or reducing the work of breathing. Additionally, high flow therapy may generate a flushing effect in the nasopharynx such that the anatomical dead space of the upper airways is flushed by the high incoming gas flows. This creates a reservoir of fresh gas available for each and every breath, while reducing re-breathing of carbon dioxide, nitrogen, etc.

In one example for high flow therapy, an unsealed or non-sealing user interface, e.g. a nasal cannula, is used. For CPAP or other pressure therapy a sealed interface is typically used, e.g. a nasal mask, full face mask, or nasal pillows.

The patient interface 17 may be a non-sealing interface to prevent barotrauma when the apparatus is providing high flow therapy (e.g. tissue damage to the lungs or other organs of the respiratory system due to difference in pressure relative to the atmosphere). The patient interface may be a nasal cannula with a manifold and nasal prongs, and/or a face mask, and/or a nasal pillows mask, and/or a nasal mask, and/or a tracheostomy interface, or any other suitable type of patient interface. The patient interface may comprise a headgear configured to maintain the interface on the face of the user.

As described below, the breathing assistance apparatus 10 has various features to assist with the functioning, use, and/or configuration of the breathing assistance apparatus 10.

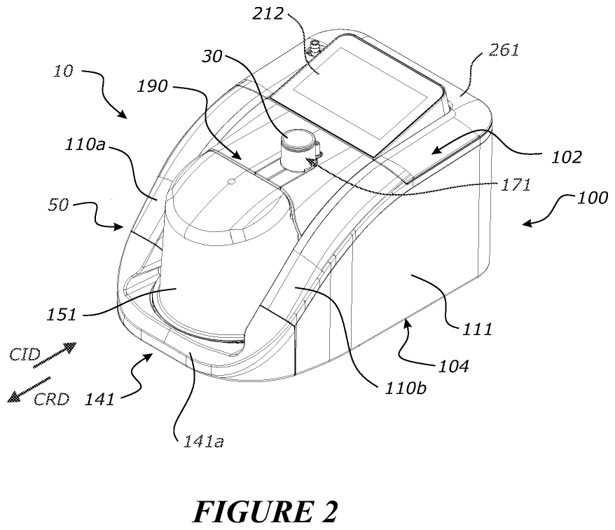

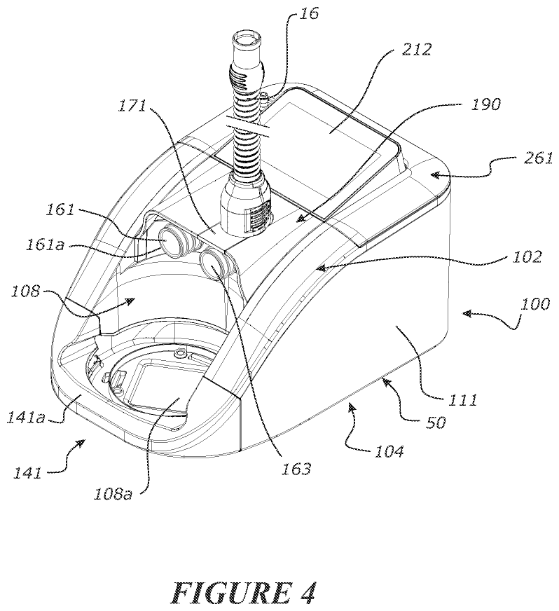

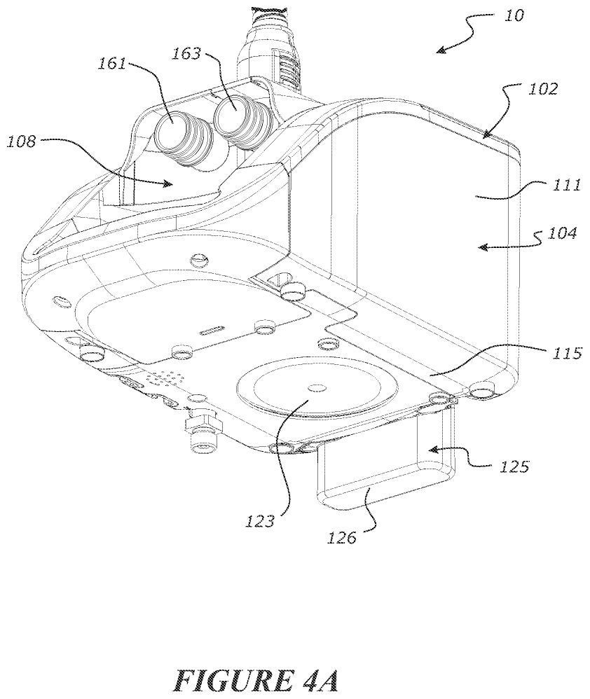

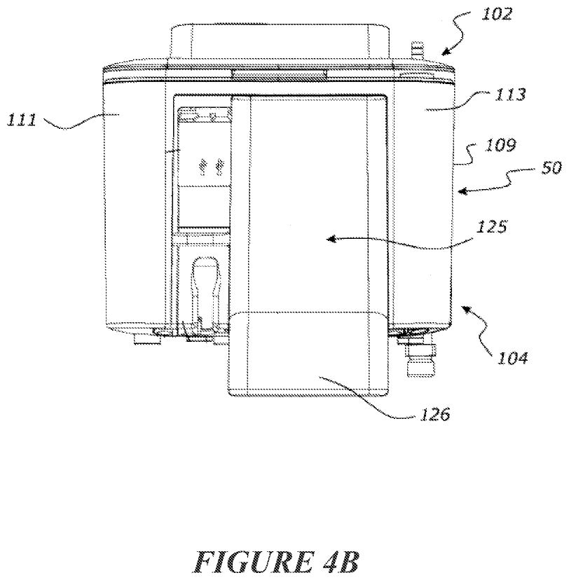

As shown in FIGS. 2-4B, a first configuration breathing assistance apparatus 10 comprises a breathing assistance apparatus base unit 50 having a main housing 100. The main housing 100 has a main housing upper chassis 102 and a main housing lower chassis 104.

The main housing of the base unit 50 has a peripheral wall arrangement. The peripheral wall arrangement defines a recess 108 that provides a humidifier liquid chamber bay for receipt of a removable liquid chamber 151. The removable liquid chamber 151 contains a suitable liquid such as water for humidifying gases that will be delivered to a patient. The base unit 50 of the apparatus 10 may have a movable finger guard 141 that guards against a user touching a base flange 155 of the liquid chamber when the liquid chamber is in place in the recess 108 and when a barrier 141a of the finger guard is in a covering position as shown in FIG. 2. The barrier 141a is movable between the covering position and a lowered access position in which the recess 108 is less covered or is uncovered by the barrier 141a.

In the form shown, the main housing lower chassis 104 peripheral wall arrangement comprises a substantially vertical left side outer wall 109 that is oriented in a front-to-rear direction of the main housing 100, a substantially vertical right side outer wall 111, and a substantially vertical rear outer wall that extends between and connects the walls 109, 111. A bottom wall 115 extends between and connects the lower ends of walls 109, 111, 113, and forms a base of the apparatus and a substantially horizontal floor portion of the liquid chamber bay.

The floor portion of the recess 108 has a receptacle portion 108a to receive a heater arrangement such as a heater plate 140 or other suitable heating element(s) for heating liquid in the liquid chamber 151 for use during a humidification process. The heater plate would typically have a shape that substantially corresponds to the shape of a base 154 of the liquid chamber 151, such as a circular shape for example. The heater plate 140 is resiliently mounted; for example, on biasing device(s) such as spring(s). The resilient mounting enables the heater plate to move downwardly to accommodate the liquid chamber 151 in the recess 108, while maintaining good contact between the heater plate 140 and the base of the liquid chamber once the liquid chamber is inserted in the recess 108.

The main housing lower chassis 104 is attachable to the upper chassis 102, either by suitable fasteners or integrated attachment features such as clips for example. When the main housing lower chassis 104 is attached to the main housing upper chassis 102, the walls of the upper and lower chassis engage with each other.

The lower chassis 104 has a motor recess for receipt of a motor module which may be permanently inserted in the recess or may be removable from the recess. A recess opening is provided in the bottom wall 115 adjacent a rear edge thereof, for receipt of the removable motor module. A base 123 of the motor module covers the opening into the motor recess. The base may be fixed after assembling the base to lock the motor module within the motor recess to prevent tampering with the motor. The motor module comprises a motor that forms a blower to cause gas flow, and may comprise one or more sensors to sense properties of the gas passing through the motor module. The motor module may comprise sensor(s) to sense parameters of gases flowing through the motor module. In one example the motor module may comprise a sensing module that supports a plurality of sensors e.g. a flow sensor, a differential pressure sensor, a gas composition sensor, a humidity sensor and/or any other sensors. The sensors are arranged to be in electronic communication with the controller such that the controller can receive sensor outputs to be used by the controller during control of the apparatus and its components.

The motor module and housing of the base unit 50 of the apparatus 10 are provided with suitable tubes and/or gas flow passages to deliver gases from one or more gases inlets of the base unit 50 of the apparatus, to a gas inlet port 157 of the liquid chamber 151 to humidify the gases. The gases are delivered from a gas outlet port 159 of the liquid chamber 151 to the patient outlet port 30 (via a humidified gas inlet port 163) and thereby to the patient via the patient breathing conduit 16 and patient interface 17.

The housing may comprise two gases inlets 27, 28. The first inlet may be an ambient air inlet and the second inlet may be for a supplementary gas e.g. oxygen or heliox or another supplementary gas. In the illustrated example the supplementary gas is oxygen. The supplementary gas source may comprise a valve that is controlled by the controller 13 to regulate the amount of supplementary gases introduced into the apparatus 10. The air and supplementary gases are mixed by the flow generator i.e. the blower.

The motor recess comprises a recess opening in a bottom wall 115 of the housing. Alternatively, the recess opening could be in a different part of the housing, such as a side, front, or top of the housing.

The base unit 50 of the apparatus 10 may have a battery module 125 to provide power to the apparatus when there is a power outage or for portable use. The battery module comprises a battery cover 126 containing a battery. The battery of the battery module 125 may be replaceable.

The battery module 125 may provide power if mains power is disconnected. In some embodiments the controller is configured to detect disconnection of mains power has and automatically switch to draw power from the battery module 125 to provide functions of the breathing assistance apparatus.

When the battery module 125 is utilized to power the apparatus, the apparatus may operate for a specific amount of time e.g. 30 minutes to 1 hour.

In the form shown, the battery cover 126 of the battery module 125 is coupled to an exterior of the back wall 113 of the apparatus housing 100. This provides a large surface area to cool the battery and reduces the amount of heat entering the apparatus from the battery. Additionally, this configuration reduces the influence of heat generated by components of the apparatus on the battery, particularly when the battery is being charged. In an alternative configuration, the battery may be internally mounted in the main housing.

The housing may be provided with a battery cover 126 to cover the battery once installed. Alternatively, the battery may mount directly to the housing 100 without a cover. The battery, and therefore the battery cover 126, may be sized to not extend beyond the bottom wall 115 of the housing. Alternatively, the battery cover 126 may be longer and extend beyond the bottom wall 115 of the housing to accommodate a larger battery.

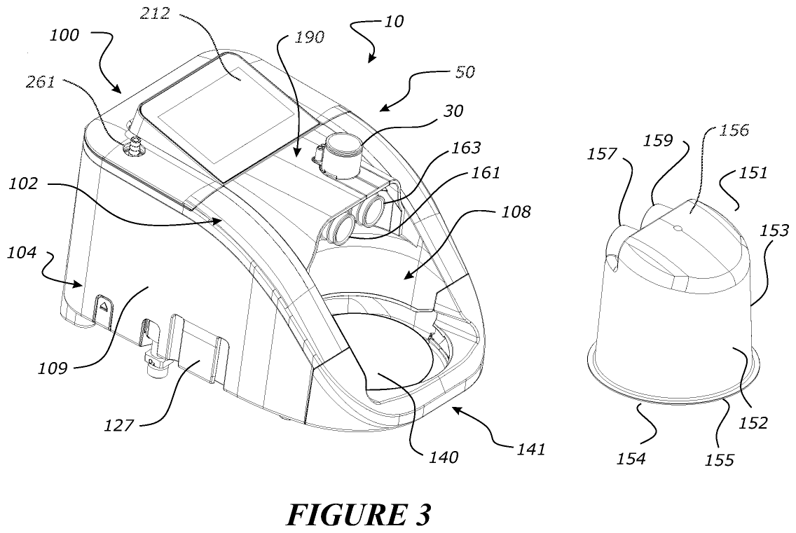

As shown in FIG. 3, the base unit 50 of the apparatus 10 has a mounting feature 127 for mounting the apparatus to a support apparatus.

The mounting feature 127 may be integrally formed with part of the main housing of the base unit 50 of the apparatus 10. In the form shown, the mounting feature 127 is integrally formed with the left side wall 109 the lower chassis 104 of the housing. The mounting feature 127 could instead be integrally formed with any of the other walls of the housing, such as a rear wall, right side wall, or other wall.

The main housing of the apparatus may be formed from any suitable material that will allow the mounting feature 127 to be integrally formed. For example, the housing may be formed from polycarbonate.

FIG. 3 shows a humidifier liquid chamber 151 (i.e. a reservoir) for use with the breathing assistance apparatus 10. The chamber 151 is a removable liquid chamber to be filled with liquid such as water for the humidification of respiratory gases. The liquid chamber 151 is removable from the base unit 50 of the breathing assistance apparatus 10 to be more easily re-filled or disposed of.

The liquid chamber 151 has a body 152 having a peripheral wall 153 and a roof 156. The body defines an internal chamber for receipt of a liquid. A base 154 is provided at the lower end of the peripheral wall, and comprises a base flange 155 that projects outwardly from the lower end of the peripheral wall 153. First and second base unit connection ports comprising a liquid chamber gas inlet port 157 and a liquid chamber gas outlet port 159 are in communication with the internal chamber of the liquid chamber 151. The breathing assistance apparatus base unit 50 comprises complementary chamber connection ports comprising a gas outlet port 161 and a humidified gas inlet port 163. When the liquid chamber is received in the recess 108 to engage with the housing 100, the liquid chamber gas inlet port 157 connects to the gas outlet port 161 that receives gases from the motor module via a gas flow passage, and the liquid chamber gas outlet port 157 connects to the humidified gas inlet port 163 to deliver humidified gases from the liquid chamber to the patient outlet port 30.

The liquid chamber could have a generally circular peripheral shape, or could be any other suitable shape, with the recess 108 shape modified accordingly if required.

In the form shown, the liquid chamber 151 has a substantially cylindrical shape.

The base 154 of the liquid chamber is heat conductive. In particular, the base 154 of the liquid chamber 151 is made from a highly heat conductive material, which allows heating of the liquid in the chamber when in contact with the heater plate 140 of the base unit 50 of the breathing assistance apparatus 10 during use.

The liquid chamber 151 can be fluidly coupled to the base unit 50 of apparatus 10 in a rearward insertion direction of the liquid chamber 151 into the recess 108, from a position at the front of the housing 100 in a direction toward the rear of the housing 100. The gas outlet port 161 is in fluid communication, via a fixed L shaped elbow, with a gas flow passage from the motor/impeller unit.

The humidified gas inlet port 163 is embodied in a removable component comprising removable elbow 171 that can be removably connected to the housing. The removable elbow 171 is L-shaped, and further comprises the upstanding patient outlet port 30 for coupling to the patient breathing conduit 16 to deliver gases to the patient interface 17. In different configurations, the removable component may not have an elbow shape, and could instead, for example, have aligned inlet and outlet ports.

The gas inlet port 157 of the liquid chamber is complementary with the gas outlet port 161 of the breathing assistance apparatus base unit 50, and the gas outlet port 159 of the liquid chamber is complementary with the humidified gas inlet port 163 if the breathing assistance apparatus base unit 50. The axes of those ports may be parallel and/or horizontal enable the liquid chamber 151 to be inserted into the recess 108 in a substantially linear movement to form gas connections between the ports.

The chamber connection ports 161, 163 are parallel cylindrical features extending from the housing of the breathing assistance apparatus base unit 50.

In some embodiments, the base unit connection ports 157, 159 of the humidification chamber (for example the liquid chamber) are pneumatically connected to the chamber connection ports 161, 163 of the apparatus 50 when the humidification chamber is provided to the liquid chamber bay (for example as described in more detail above).

The chamber connection ports 161, 163 (which in the form shown are male connection members) of the breathing assistance apparatus base unit 50 insert into the base unit connection ports 157, 159 (which in the form shown are female connection members) of the liquid chamber in a concentric manner. The recess 108 may comprise one or more guide rails to assist with holding the liquid chamber in position in the recess 108.

The breathing assistance apparatus 10 may have any one or more of the features and/or functionality of the breathing assistance apparatus described and shown in PCT Patent Application Publication WO2016/207838, filed 24 Jun. 2016. The contents of that specification are incorporated herein by way of reference.

In order to prevent gas leaking from either of the two connections (port 157 to port 161, and port 159 to port 163), one or more sealing elements are provided for each connection. The one or more sealing elements may be on the outer surface of male ports, and seal against the inner surface of female ports. In one configuration, the gas inlet port 157 of the liquid chamber and the gas outlet port 159 of the liquid chamber are the female ports, and the housing ports, i.e. the gas outlet port 161 and the humidified gas inlet port 163 are the male ports. Alternatively, the ports 157, 159 of the liquid chamber may be the male ports and the ports 161, 163 of the breathing assistance apparatus base unit 50 may be the female ports.

The screen 212 (refer to FIGS. 2 and 3) is located on the housing (e.g., upper side, lateral side) and is positionally fixed. However, the screen 212 can be positionally adjustable (e.g., hingedly). The screen may also be referred to as a ‘display’.

The screen 212 as shown in FIGS. 2 and 3 is rectangular, but can be shaped differently (e.g., square, circular). The screen 212 is mains or battery powered. The screen 212 is located on an upper surface of the housing and is also angled to improve visibility of the screen to a patient. Further the screen 212 being located on the upper surface of the housing makes the screen and the screen contents easier to view by a patient (e.g. within a home environment).

In some embodiments the screen 212 is a colour screen, and preferably a colour touchscreen (e.g., resistive, capacitive). The screen 212 is large in size which makes it easier to view content presented on the screen for patients. These patients often can be quite ill e.g. COPD patients and can often be elderly. Having a large, high resolution screen 212 helps to easily convey the inquiry and the content of the inquiry to the patient. It makes the inquiry more engaging since it is easier to view. The large touchscreen 212 also improves legibility of the presented content which further helps to make the content more engaging.

The large resolution and the colour touchscreen 212 allows presentation of various queries that may require colours or shades of colours to be presented (for example sputum colour). The screen 212 being a touchscreen also allows for easier interaction with the patient as the touchscreen is simpler to use and more intuitive to use since user is required to touch the screen 212 and perform gestures on the screen to input information. The touchscreen avoids the need to use buttons or dials that may require a complex sequence to be pushed to input data e.g. respond to the content of the inquiry.

The resolution of the screen 212 is sufficiently large to make the presented content easy to read and easy to interact with. This may be of particular importance for old patients, and/or unwell patients with comorbidities where a large screen more clearly presents the queries and answers relative to a smaller screen.

In one example the touchscreen comprises a resolution is at least 300×150 pixels. In one example the touchscreen comprises a resolution of 400×250 pixels. More preferably the screen comprises a resolution of 480×272 pixels.

The touchscreen may have a resolution of at least 600×400 pixels

The touchscreen may be at least 3.5 inches in diagonal measurement.

The touchscreen may be at least 4 inches in diagonal measurement.

The touchscreen may be 4.2 inches in diagonal measurement.

The touchscreen may be up to 7 inches in diagonal measurement.

The touchscreen may be an OLED or TFT LCD screen.

In an alternative form, the user interface may comprise a colour touchscreen or display along with a plurality of operable input elements such as buttons, dials, or switches. The input elements may be mechanical and/or touch-sensitive elements. The touchscreen in combination with the operable input elements allows a patient to input information using a combination of buttons or switches or dials and the touchscreen.

The removable elbow 171 is removable from the housing 100 when the shroud 190 is attached to the housing. The breathing assistance apparatus 10 may comprise the features of a breathing assistance apparatus, in particular a high flow apparatus as described in PCT Patent Application Publication WO2020/095186, filed 5 Nov. 2019, the contents of this specification are incorporated herein in its entirety by way of reference.

The apparatus may comprise a valve to allow supplementary gases to be introduced to the blower.

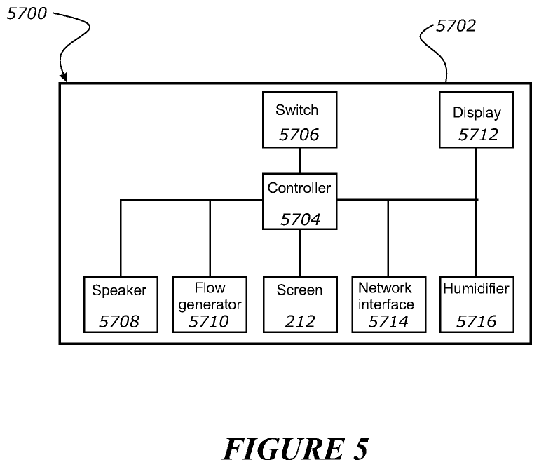

FIG. 5 is a schematic of a breathing assistance apparatus 5700. In particular, a breathing assistance apparatus (the breathing assistance apparatus may be the breathing assistance apparatus 10 as described above) can be similar to other respiratory or breathing apparatuses described above. The breathing assistance apparatus includes a housing 5702 (e.g., housing 100) including a controller 5704 (e.g., controller 13), a switch 5706, a speaker 5708, a flow generator 5710 (e.g., flow generator 11), a display 5712 (e.g., touch-enabled screen 212), a network interface 5714 (e.g., transmitter and/or receiver 15), and a humidifier 5716 (e.g., humidifier 12).

As described above in context of FIGS. 1 to 4B, the housing 5702 has a fluid inlet and a fluid outlet. Likewise, the flow generator 5710 is located within the housing 5702 downstream of and in fluid communication with the fluid inlet, whether mains or battery powered. Similarly, the humidifier 5716 is located within the housing 5702 downstream of and in fluid communication with the flow generator 5710 and upstream of and in fluid communication with the fluid outlet, whether mains or battery powered. Moreover, the humidifier 5716 includes a heater (e.g., heating plate, heating element), whether mains or battery powered.

The controller is in electronic communication with the flow generator, the display, the network interface, and the heater plate. The controller comprising an electronic processor (e.g., logic controller, multicore processor) and a non-transitory memory (e.g., flash memory) in communication with the electronic processor. The controller controls activation/deactivation and operation of the flow generator 5710, the heater plate of the humidifier 5716, and the screen 212.

The switch 5706 (e.g., analog, digital) is located on the housing 5702, but can be located not on the housing 5702 (e.g., on power cord), or can be incorporated as a software implement switch on, (e.g., screen 212). The switch 5706 is a power switch electrically and/or mechanically coupled to the controller 5704 and that switches between an on-mode and an off-mode, which can be based on a manual input (e.g., user input, patient input). The breathing assistance apparatus 5700 can be activated based on the switch 5706 switching from the off-mode to the on-mode and deactivated based on the switch 5706 switching from the on-mode to the off-mode. The switch 5706 can be embodied in many physical, electronic, or virtual ways. For example, the switch 5706 can be embodied as a physical or virtual button, a knob, a dial, a rocker, a toggle, or a lever. The switch 5706 can be omitted.

The speaker 5708 is located on the housing 5702 and is configured to output a sound content (e.g., tones, speech, music). The speaker 5708 can be mono or stereo. The speaker 5708 can be mains or battery powered. In some embodiments the speaker 5708 is not present.

The display 5712 (for example screen 212) is located on the housing 5702 (e.g., upper side, lateral side) and is positionally fixed. However, the display 5712 can be positionally adjustable (e.g., hingedly). The screen 212 can be grayscale or color. The screen 212 can be a touchscreen (e.g., resistive, capacitive). The screen 212 is rectangular, but can be shaped differently (e.g., square, circular). The screen 212 is mains or battery powered.

In some embodiments the apparatus allows for insertion of a USB or memory storage device. This allows for data (e.g. responses to the queries) to be downloaded and then plugged into a computer, such as a PC or laptop. The PC or laptop can then be used to transmit responses to the patient and device management platform e.g. a server for patient and device management.

In some embodiments the apparatus comprises a network interface 5714. As will be explained, the network interface comprises one or more communication modules that enable data communication or a data link to one or more external devices, systems, or servers. The network interface is located within the housing, but can be located externally on the housing. The network interface may include one or more of a wireless signal receiver, a wireless signal transmitter, and a wireless signal transceiver, each having a wireless signal radio antenna or a light signal modulator depending on signal modality (e.g., radio, light), although wired communication is possible (e.g., wired network card). The network interface is configured to communicate on a Wi-Fi, Li-Fi, Bluetooth, ZigBee, Z-Wave, cellular, or a satellite network, whether via a local, wide, personal or other area networks. For example, the network interface can communicate with a patient and device management platform (e.g. a server) or a computing device (e.g., smartphone, tablet, wearable, medical monitor) that is local or remote to the network interface. Note that the network interface can include a first transceiver of a first modality (e.g., Wi-Fi) and a second transceiver of a second modality (e.g., Bluetooth). For example, the network interface can include a cellular modem (3G, 4G, 5G, 6G), a Wi-Fi card, and a Bluetooth chip. In some embodiments the network interface is not present.

The memory of the controller includes instructions executable by the electronic processor that when executed by the electronic processor cause the controller to perform various operations, as further described below.

For example, the instructions can cause the controller to (a) activate the heater plate upon activation of the breathing assistance apparatus, (b) request the screen 212 to display a user interface presenting a plurality of user health queries (e.g., relating to patient disease progression or patient health condition) and a plurality of user input elements via which user inputs are received, (c) refrain from activating or prevent activation of the flow generator until a predetermined plurality of the user inputs have been received, and (d) send the predetermined plurality of user inputs to a patient and device management platform (e.g. a server, web application, database, virtual server, cloud service) via the network interface. The controller can receive the predetermined plurality of user inputs before the heater plate of the humidifier reaches a predetermined temperature (e.g., about 37 degrees temperature of gas). The user health queries and the user input elements can be displayed upon booting of the controller.

For example, the instructions can cause the controller to (a) request the screen 212 to display a user interface presenting a plurality of requests for user health information and a plurality of user input elements via which the user health information is received by the controller as user inputs. The requests for user health information and the user input elements can be displayed upon booting of the controller. The predetermined plurality of the user inputs can be all of the user inputs.

The controller may be configured to communicate two or more queries sequentially to the screen 212, the screen 212 presenting the two or more queries sequentially to the user. After receiving a response to each presented query at the screen 212, the screen 212 is configured to communicate each response to the controller.

The controller may also be configured to lock access to any other modes, functions until the controller receives a response to each query. Alternately, the controller may restrict access to an operative mode until the controller receives response to each query. Alternately, the controller may be configured to disable operation of the flow generator and heater plate until a response to each query is received. However, the controller may allow a user bypass responding to the enquiry or may automatically bypass presentation of the enquiry if the user (e.g., patient) has responded to the enquiry at least once in a day.

### 3. Examples of Data System and Method

### 3.1 Overview of Data Capture by Breathing Assistance Apparatus

The breathing assistance apparatus is configured to deliver a personal health enquiry to the patient or user. The personal health enquiry is a digital questionnaire comprising a set of queries presented to the user on the display screen of the breathing assistance apparatus, and which provides response options for the patient. The queries relate to a series of health parameters of the patient. The responses entered by the patient (e.g. via interaction with the user interface, e.g. touchscreen GUI or other user input element) is captured as the answer data for the personal health enquiry. Typically, this personal health enquiry is captured or presented on a periodic basis according to a time interval frequency, e.g. daily, weekly or some other configurable time interval.

In one configuration, the queries may be categorized into group. In one configuration there are queries in a symptoms category relating to the patients respiratory or related symptoms, and queries in a medication category relating to how much medication a patient is using. The answer data may comprise symptom data representing the answers to the symptom queries, and medication data representing the answers to medication queries. The captured answer data is then sent or transmitted to a data server for processing, as will be explained in further detail later.

In some embodiments, each time a patient activates the breathing assistance apparatus, the apparatus can present a series of queries on one or more touch screens.

The queries form part of the enquiry (i.e. enquiry form). The breathing assistance apparatus records the patient's answers to the queries. In one embodiment the query pertains to a “health parameter”. In some embodiments the query relates to non-health related parameters.

In some embodiments the health parameter can be a subjective factor related to a patient's perception of their health. For example, this may be a patient's general feeling.

In some embodiments, the health parameter may be based on a patient parameter (for example a parameter i.e. respiratory rate, oxygen saturation of the patient measured by the apparatus as described in more detail below.

The health parameter may be a subjective qualitative factor based on a patient's perception of their health such as throat soreness, degree of breathing difficulty and severity and/or type and/or frequency of coughing. The health parameter may be an objective quantitative factor that is directly measureable such as sputum colour (based on a colour chart), medicine use (e.g. frequency and amount of medicine such as for example antibiotic, steroid or inhaler).

The health parameter may be determined based on the answer to a single query, or based on the answers at least two queries.

At least some of the health parameters are indicative of COPD symptoms that a COPD patient can often suffer. COPD is chronic obstructive pulmonary disease. COPD is a respiratory disease characterised by inflammation of the airways. At least some of the health parameters may be indicative of other obstructive pulmonary diseases. At least some of the health parameters may also be indicative of dyspnea and/or respiratory distress, and/or bronchiectasis.

The breathing assistance apparatus may comprise one or more touch screens which display after the apparatus is turned on (e.g. booting). In some embodiments the, or each, touch screen displays a query. Alternately, the query may be implemented as multiple pages that can be scrolled through. Alternately, the query may be presented as a single document that is scrolled through to respond to the queries. Alternately each screen displays a separate query, and each time the patient answers a query, the next query is presented on the screen.

In some embodiments the queries are displayed on a user I/O interface (as described in more detail below).

In some embodiments, the breathing assistance apparatus may comprise an internal clock, the internal clock may maintain the current date and time. The internal clock may be part of the controller of the breathing assistance apparatus, and/or part of the patient and device management platform.

The enquiry may be presented based on the current date and time. For example, the enquiry may be presented once daily or weekly based on information from the internal clock, or any other configurable frequency set by a healthcare provider for example.

In one example implementation the enquiry is presented once when the device is switched ON.

The enquiry may be presented at an initial use each day (for example each day of the week), or each predetermined time period.

In some embodiments the internal clock is a real-time clock (RTC).

In some embodiments, after the patient answers the last query of the enquiry, the breathing assistance apparatus displays a therapy control screen, which the patient may use to initiate therapy.

In some embodiments, the patient may skip the enquiry if, for example, they are feeling too unwell to complete it. If the patient skips the enquiry, the device will enable therapy to begin without the need for the patient to answer the enquiry. For example, the touch screen will display a therapy control screen, which the patient may use to initiate therapy.

The therapy control screen may present multiple selectable operative parameters of the breathing assistance apparatus e.g. a flow rate or a temperature of gases provided to the patient (i.e. a patient end temperature), an 02 concentration or other such operative parameters.

The patient may be required to complete the enquiry (for example comprising a number of queries) and respond to each query of the enquiry presented on the screen of the breathing assistance apparatus. The patient may be able to skip the enquiry if the patient has completed the enquiry at least once per day.

In some embodiments, the controller may keep track of the answers to the queries from the patient. After all the queries of the enquiry are answered, the controller may determine the enquiry to be complete.

As described above after the patient answers the last query of the enquiry the controller may determine the enquiry complete.

In some embodiments the enquiry is presented on the touch screen while the breathing assistance apparatus is setting up.

### 3.2 Controller Operation to Present Health Enquiry and Capture Answer Data

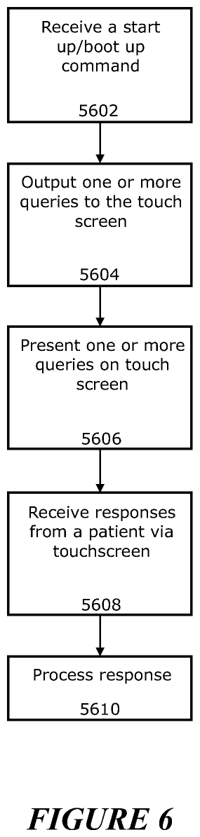

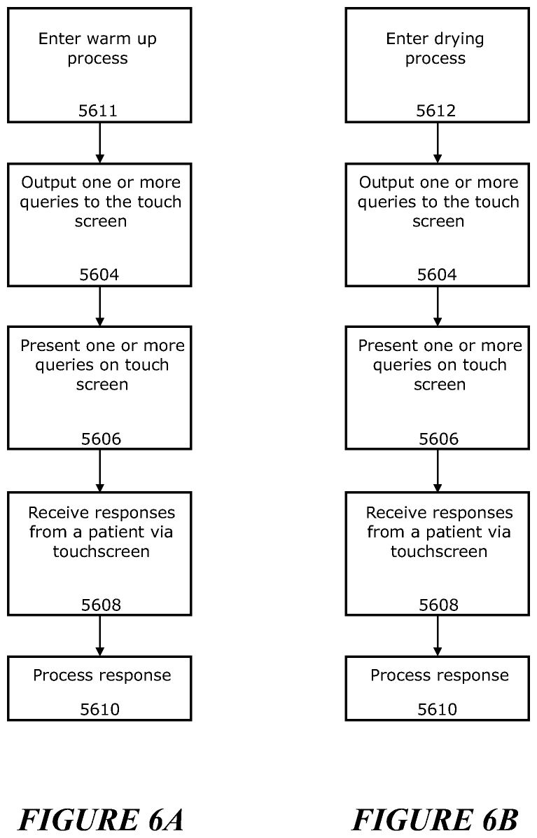

FIG. 6 is a flowchart for the controller operation. As shown in block 5602 of FIG. 6 the breathing assistance apparatus may receive a start-up/boot up command which initiates the device. The start-up/boot up command may be received by the apparatus via the user interface of the screen 212. In some embodiments, when power is provided to the apparatus the screen is configured to be activated, so that the user can interact with the screen. Additionally or alternatively, the start-up/boot up command may be received by the apparatus via a switch (for example a button).

This start-up can include booting of the breathing assistance apparatus or activation of the breathing assistance apparatus. As indicated by block 5604, the start-up/boot up activates the apparatus to initiate the enquiry to the user which leads to one or more queries being output to the screen 212 so that the one or more queries are presented on the touchscreen for the patient (see block 5606).

In some embodiments, entering a warm-up mode and/or a drying mode of the breathing assistance apparatus activates the apparatus to initiate the enquiry to the user.

In some embodiments, as described in more detail below the start-up may include presenting a therapy control screen, which the patient may use to initiate therapy.

The queries can have a first query and a second query, where the first query precedes the second query. Correspondingly, the potential answers include a first potential answer input and a second potential answer, where the first potential answer precedes the second potential answer and the first potential answer corresponds to the first query and the second potential answer corresponds to the second query. As such, the second query can be content-dependent on the first answer, whether the first potential answer and the second potential answer are from a same user session or not (e.g., the first potential answer is from a first user session and the second potential answer is from a second user session, where the first user session precedes the second user session). For example, this content dependency can be based on a for loop, a while loop, a counter, if-then logic tree, or other logical expressions, whether the second query is retrieved or generated from the memory of the breathing assistance apparatus 5700 (the breathing assistance apparatus 5700 may be the breathing assistance apparatus 10 as described above) or from the patient and device management platform (e.g. a server in communication with the breathing assistance apparatus 5700).

In some embodiments, the answer to an earlier query (for example the first query) may determine the content of a subsequent query (for example the second query). For example, the answer to earlier queries may update the particular queries in the health enquiry, and/or the update order of the queries in the queries in the health enquiry.

In some embodiments, after a query is answered (for example a first query) a health parameter may be updated, the determination of the content of a subsequent query (for example the second query) may be based on the health parameter (for example the updated health parameter).

The health provider (for example a doctor) may be able to generate a health enquiry for a patient by selecting one or more predetermined queries from a database of queries.

The database of queries may be presented to the health provider as a list (for example a tick box interface) and the health provider can select a number of queries from the database of queries to form the enquiry.

The database of queries may be located on the patient and device management platform and/or the apparatus.

In some embodiments the database of queries may be customised based on a worsening of one or more symptoms. For example, if a patient is indicating a worsening in symptoms related to sputum colour the database of queries may be customised to include queries related to upper airway health.

If the health provider (for example a doctor) is selecting the one or more predetermined queries from a database of queries remotely (i.e. not at the apparatus) once the healthcare provider selects the queries, the selected queries may be transmitted to the apparatus, and stored on the device.

In some embodiments, the apparatus may periodically communicate with the patient and device management platform to obtain any updates to the enquiry and/or any queries. Additionally, or alternatively, the patient and device management platform may communicate with the apparatus to notify the apparatus that updates to the enquiry and/or any queries are available, and transmitted the updated enquiry and/or queries.

In some embodiments the queries (as part of the health enquiry) may be based on a patient condition (for example COPD, bronchiectasis etc). Queries may be added to or removed from the health enquiry based on the patient condition. For example, if the patient has bronchiectasis then the health enquiry may include an enquiry relating to the colour of sputum and whether the patient is taking antibiotics.

The health provider (for example a doctor) may select queries from a list relating to a patient condition to be added to the health enquiry. Alternatively, or additionally, the health provider (for example a doctor) may select a patient condition and the health enquiry may be automatically updated based on the selection. In some embodiments, the database of queries may be customised based on a patient condition (for example chronic obstructive pulmonary disease (COPD) or respiratory distress syndrome or dyspnea, or bronchiectasis etc.) For example, if the patient has COPD the database of queries may be customised to include queries relating to COPD.

In some embodiments, the queries of the health enquiry are ordered such that queries related COPD condition are displayed first, followed by queries related to a bronchiectasis condition.

In some embodiments, general health queries are presented before queries related to a COPD condition and queries related to a bronchiectasis condition.

For example, queries relating to COPD may include queries about daily sputum production, sputum colour, and/or coughing.

For example, queries relating to bronchiectasis may include queries about steroid usage, and/or inhaler usage.

In some embodiments the specific condition (for example COPD or bronchiectasis) of the patient may be determined based on the answers to queries relating to the specific condition. For example, if the answers to the queries relating to bronchiectasis are below a baseline and/or worsening this may be indicative of the patient having bronchiectasis.

The health provider (for example a doctor) may be able to add custom queries to the health enquiry. The custom enquires may include custom questions and associated answers relating to information the doctor may wish to keep track of.

The health provider (for example a doctor) may be able to add custom queries to the database of queries.

The health provider may generate the health enquiry directly on the apparatus or from an ancillary device (such as a mobile device connected to the patient and device management platform).

The patient then enters their response via the touchscreen to each query (see block 5608). The patient's responses (answer data) are then processed 5910. This processing may include plotting or graphing the set of the responses, as will be explained later, and/or sending the data set of responses (answer data) to a patient and device management platform, external storage, mobile device, or insurance, equipment or healthcare provider. Analysis or processing of the data set (answer data) can be performed at any stage, for example, by the patient and device management platform. In some embodiments, after the patient answers the last query of the enquiry, the breathing assistance apparatus displays a therapy control screen, which the patient may use to initiate therapy.

In some embodiments, the patient and device management platform can store a patient profile that comprises one or more of: patient details, baseline data of the health parameters of a patient, serial number of the patient's breathing assistance apparatus and/or prescription settings.

### 3.3 Health Enquiry During Warm-Up Process

In some embodiments, for example as shown in FIG. 6A the enquiry is presented to the user for example on the touch screen during a warm-up process 5611.

The warm-up process comprises activating the heater plate of the breathing assistance apparatus to warm-up the contents of the humidification chamber.

The warm-up process may comprise warming the heater plate of the breathing assistance apparatus to specific temperature. For example, the specific temperature may be a predetermined temperature (for example a standby temperature). The predetermined temperature may be less that an operational temperature of the heater plate when the humidifier is providing humidification. The specific temperature may be about 35 degrees Celsius.

Controlling the heater plate comprises controlling a power provided to the heater plate. The apparatus heater plate may comprise a heater plate temperature sensor, and the controller may control the heater plate temperature to the specific temperature of the warm-up process via, for example, closed loop control.

In some embodiments, the warm-up process may comprise warming the heater plate of the breathing assistance apparatus to achieve a specific temperature.

In some embodiments, the warm-up process comprises warming the heater plate of the breathing assistance apparatus to control the chamber outlet temperature to a specific temperature.

The chamber outlet as described elsewhere is the outlet of a humidification chamber, and optionally measured in an elbow located after the chamber outlet of the humidification chamber.

The specific temperature may be based on one or more temperature set points (as one or more operative parameters of the apparatus for therapy) of the apparatus. The set points may be entered by the user via the therapy control screen as described elsewhere, for example a desired chamber outlet temperature, a desired dew point temperature (chamber outlet or at the end of the breathing conduit), or a desired patient end temperature (at the end of the breathing conduit.)

One or more temperature set points may correspond to a required relative humidity or a required absolute humidity, optionally the relative humidity is about 90% to about 100% or is about 100%.

In some embodiments, the warm-up process may comprise warming the heater plate of the breathing assistance apparatus to achieve a humidity of the gases (for example at the chamber outlet or at the end of the breathing conduit).

The apparatus can provide an audible or visual indication e.g. a visual indicia once the warm-up process is completed.

In some embodiments the warm-up process may comprise running the flow generator (for example a blower) at a predetermined flow rate or a predetermined flow generator output (for example a predetermined motor speed). During the warm-up process no therapeutic flow is being provided to the patient.

The enquiry and the queries comprising the enquiry are presented on the touchscreen (or other user I/O interface) of the breathing assistance apparatus during the warm-up process (for example as shown in FIG. 6A). This advantageous because the patient is often waiting until the warm-up process is completed.

The warm-up process can be several minutes e.g. between 5 mins to 25 mins. This provides a suitable time period where flow therapy is not being used by the patient, thereby allowing the patient free time to respond to the enquiry (i.e. respond to the queries presented on the touch screen).

Presenting the queries to the user during the warm-up process allows the patient to use the time where usually they would be waiting for the machine to warm-up to answer the queries. Utilising the warm-up process to present the queries may increase the likelihood of the patient answering the queries.

In some embodiments, the therapy control screen may be presented on start-up of the device.

In some embodiments, the warm-up process may be activated on start-up of the device.

In some embodiments, the warm-up process may be manually activated by a user.

In some embodiments, the warm-up process is activated once the user has initiated therapy (for example via the therapy control screen).

In some embodiments, the breathing assistance apparatus displays a therapy control screen on start-up, which the patient may use to initiate therapy (as described above). Once the user has entered one or more operative parameters of the breathing assistance apparatus on the therapy control screen (e.g. a flow rate or a temperature of gases provided to the patient (i.e. a patient end temperature), an 02 concentration or other such operative parameters) the apparatus may enter the warm-up process and optionally display the enquiry (for example as described above).

### 3.4 Health Enquiry During Drying Process

In some embodiments, for example as shown in FIG. 6B the enquiry is presented to the user for example on the touch screen during a drying process 5612.

The drying process may be configured to evaporate remaining condensate in the apparatus and/or patient breathing conduit and/or patient interface.

The drying process may be as described in PCT Patent Application Publication WO2006/126900, filed 26 May 2006. The contents of that specification are incorporated herein in its entirety by way of reference.

In one embodiment, a safety message or notification is presented on the display screen of the breathing assistance apparatus prior and/or during drying mode. The safety message may indicate to the patient or user to not put on the user interface (e.g. nasal cannula) during drying mode, i.e. do not use apparatus for respiratory therapy during drying mode. In one configuration, the safety message or notification may comprise an acknowledgement button or tab or icon or GUI element that is presented or available to the user to acknowledge the message or notification. By acknowledging the message with a response, the user is confirming that the cannula is off, i.e. not being used. Once the safety message is acknowledged by the user, the personal health enquiry may be presented to the user on the display for example.

The drying process may comprise activating the heater wire 16a in the patient breathing conduit 16 while the flow generator provides gases as a set flow rate or predetermined motor speed. The set flow rate may be lower than a therapeutic flow being provided to the patient. The predetermined motor speed may be about 1000 RPM to about 3000 RPM or less than about 2000 RPM.

Additionally, or alternatively, the drying process may comprise controlling the heater plate of the humidifier to a predetermined value. The predetermined value may be low enough to prevent the generation of humidity by the humidifier. Alternatively, the heater plate may be deactivated during the drying process.

The heater wire 16a may be controlled to a predetermined temperature at for example the end of the patient breathing conduit, or controlled to a predetermined duty cycle or to a predetermined power.

The predetermined duty cycle may be 100%.

The predetermined temperature may be greater than 45 degrees Celsius.

The drying process may be configured to operate for at about 20 minutes to about 40 minutes, or about 15 minutes.

The drying process may be undertaken at the end of a therapy session. The patient (or other healthcare professional) may indicate when the therapy session has ended, or in some embodiments the therapy session may have one or more conditions which are met to signal an end to the therapy session (for example a time elapsed).

The drying process may be manually activated by the patient or healthcare provider, or may be automatically activated at the end of a therapy session.

In some embodiments the end of therapy may be determined by the detection that the user has taken off the patient interface. The apparatus may detect that the user has taken off the patient interface based on a flow signal measured from the flow sensor. Optionally, the end of therapy may be determined by the detection that the user has taken off the patient interface and a predetermined period of time has elapsed.

In some embodiments, the apparatus may detect that the user has taken off the patient interface when breathing is not detected. Breathing may be detected based on a flow signal measured from the flow sensor.

### 3.5 Conditional Presentation of Health Enquiry

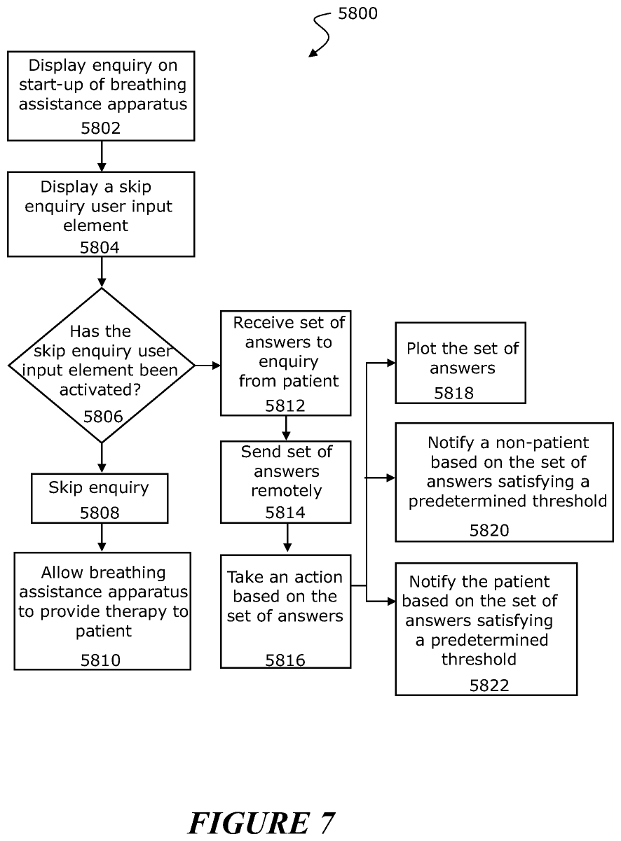

FIG. 7 is a flowchart of a process for conditionally presenting a personal health enquiry to a patient of a breathing assistance apparatus. In particular, a process 5800 is performed via the breathing assistance apparatus 5700 and the patient and device management platform, as described above. The breathing assistance apparatus 5700 may be the breathing assistance apparatus 10 as described above.

The term ‘block’ in the specification may refer to one or more steps undertaken by the apparatus 5700 (for example by the controller).

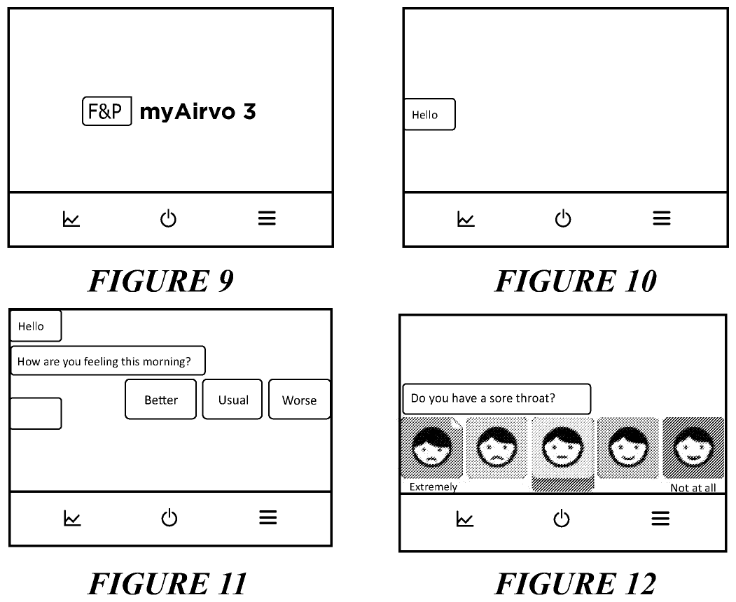

In block 5802, the breathing assistance apparatus 5700 (the breathing assistance apparatus 5700 may be the breathing assistance apparatus 10 as described above) displays a plurality of health queries, as shown in FIGS. 9-13, 13A, 14, 14A and 15-18, on the display 5712 (same as display 212) via the controller 5704 (same as controller 13) to a patient (or if the patient is incapable, a caretaker or doctor) upon start-up of the breathing assistance apparatus 5700. This start-up can include booting of the breathing assistance apparatus 5700 (e.g., controller 5704) or activation of the breathing assistance apparatus 5700 (e.g., flow generator 5710, humidifier 5716) prior to use if the breathing assistance apparatus 5700 (e.g., controller 5704) is already booted and running. In some embodiments start-up may include a warm-up process (as described above).

In some embodiments, the breathing assistance apparatus 5700 may be configured to present the personal health enquiry during a therapy session, i.e. when the apparatus is providing high flow therapy or pressure therapy to the patient.

In some embodiments, the breathing assistance apparatus 5700 may be configured to present the personal health enquiry, or prompt the patient to complete the enquiry, at least partially based on one or more patient parameters as measured by one or more sensors and/or determined by the controller of the apparatus during a therapy session. Such patient parameters may include, but are not limited to, respiratory rate, ROX index, and SpO₂ data that is measured or determined for the patient during a therapy session. In one configuration, the breathing assistance apparatus may be configured to present or initiate the personal heath enquiry based on the worsening of one or more of the measured or determined patient parameters. For example, the apparatus may be triggered to present the personal health enquiry if one or more of the patient parameters crosses one or more predetermined thresholds and/or if a worsening trend is detected. The thresholds may be general thresholds or configurable patient-specific thresholds. In some embodiments, additionally or alternatively, the worsening (e.g., relative to a threshold or trend analysis) of one or more measured or determined patient parameters may cause or trigger a change in the frequency at which the patient is presented with the health enquiry or prompted to complete the health enquiry. In some embodiments, additionally or alternatively, the worsening (e.g., relative to a threshold or trend analysis) of one or more of the measured or determined patient parameters may cause or trigger a change of the type, number and/or nature of the queries presented in the personal health enquiry.

In situations where the network interface 5714 is absent or the networking interface 5714 is unable to establish a network connection, the controller 5704 can still present the personal health enquiry and the answers can be later downloaded onto a removable memory (e.g., flash card, flash drive) or retrieved from the memory of the breathing assistance apparatus 5700 itself (e.g., maintenance). The humidifier 5716 may be the same as humidifier 12 as described earlier. The flow generator 5710 may be the same as flow generator 11 described earlier.

In block 5804, the breathing assistance apparatus 5700 (e.g., controller 5704) displays a skip enquiry user input element (e.g., graphic, text, icon) on the display 5712 via the controller 5704. Alternatively, the controller 5704 can determine whether the activation is a second or greater activation within a predetermined interval, e.g., a day, and then effect a bypass of presentation of the enquiry. Further, the controller 5704 can be programmed to request the display 5712 (e.g. a touchscreen like screen 212) to present the queries and the potential answers such that at least one of the queries or at least one of the potential answers is different between at least two instances of the flow generator 5710 being activated over a predetermined time period (e.g., at least two days).

In block 5806, the breathing assistance apparatus 5700 (e.g., controller 5704) determines (e.g., controller 5704) whether the skip enquiry user input element has been activated (e.g., via touch selection). If yes, then block 5808 is performed. If no, then block 5812 is performed. Note that this skipping functionality can be employed on a per enquiry basis (e.g., skip entire enquiry if the patient is feeling too unwell to complete it) or on a per query basis (e.g., skip specific or any queries).

Skipping information may be tracked, which itself may be a data point later identified by the patient and device management platform e.g. server. For example, a skipped enquiry (i.e. all queries in the enquiry) or a skipped query may be captured as skipped data. The skipped data may identify which queries within the enquiry were skipped or whether the entire enquiry for a time interval (e.g. specific day) was skipped. This skipped data forms part of the enquiry data for a time interval that is sent to the data server for processing. For example, the enquiry data for a daily or weekly enquiry may comprise answer data and any skipped data to the personal health enquiry.

Further, in certain situations, skipping is not enabled or is prevented. For example, if the enquiry is completed at the time the breathing assistance apparatus 5700 is first started or booted during a calendar day, then the controller 5704 will allow skipping of the query or the enquiry if the breathing assistance apparatus 5700 is switched off and started a second time in that calendar day. In some embodiments, the enquiry may need to be completed once a calendar day as controlled by the controller 5704.

In certain situations, a patient and device management platform e.g. server may send a signal to allow the controller 5704 to skip if the server has received the answers to the queries at least once a day. In certain situations, the queries may be required to be completed at predefined time intervals (e.g. every 2 days or every 3 days) or could be physician set or may be defined by clinical practice.

In block 5808, the breathing assistance apparatus 5700 (e.g., controller 5704) skips the personal health enquiry via the controller 5704 and presents a menu via the display 5712 for control (e.g., via touch selection) of the breathing assistance apparatus 5700 (e.g., flow generator 5710, humidifier 5716). For example, this allows skipping the enquiry before activating the flow generator 5710. However, the controller 5704 can be programmed to prevent or preclude skipping the enquiry before activating the flow generator 5710.

In block 5810, the breathing assistance apparatus 5700 (e.g., controller 5704) allows the flow generator 5710 or the humidifier 5716 to be used for provision of a breathing assistance apparatus to the patient. For example, the patient can operate the menu via the display 5712 to control (e.g., via touch selection) the breathing assistance apparatus 5700 (e.g., flow generator 5710, humidifier 5716).

In block 5812, the breathing assistance apparatus 5700 (e.g., controller 5704) receives a set of responses or answers (answer data) to the personal health enquiry from the patient. Such receipt can be performed via the patient touch selecting various user input elements (e.g., graphics, text, icons) displayed on the display 5712 via the controller 5704.

In block 5814, the breathing assistance apparatus 5700 (e.g., controller 5704) sends (e.g., wired, wireless, waveguide, encrypted, decrypted, unencrypted) the set of answers (answer data) via the network interface 5714 to the patient and device management platform. The patient and device management platform may be, or may be hosted by, any suitable platform for example a data server, server web application, database, cloud service, a virtual server.

The patient and device management platform is remote from the breathing assistance apparatus 5700 such that the patient and device management platform can receive the set of answers and process the set of answers, as described below. The controller 5704 can request the network interface 5714 to send the set of answers (e.g., predetermined plurality of user touch inputs) to the patient and device management platform one-by-one after each answer of the set of answers is received via the controller 5704 (e.g., answer-send followed by another answer-send). The controller 5704 can request the network interface 5714 to send the set of answers (e.g., predetermined plurality of user touch inputs) to the patient and device management platform after all of the answers (e.g., user touch inputs) are received via the controller 5714 (e.g., single send operation, single packet). The controller 5704 can request the network interface 5714 to send the set of answers (e.g., predetermined plurality of user touch inputs) to the server on a group-basis after a group of the answers is received via the controller (e.g., send after every two, three, four, five, six, seven etc. answers). Note that the controller 5704 can request the network interface 5714 to send the set of answers with a patient or machine identifier in order to enable effective identification of the set of answers and subsequent processing.

In some embodiments, the set of answers is stored in memory of the apparatus. Optionally the set of answers may be provided to the patient and device management platform for storage.

In some embodiments the apparatus may be configured to store answers, and transmit answers to the patient and device management platform once every predetermined time period (for example once a day). This may lead to lower data transmission costs relative to transmitting at a higher frequency as the data transfer required to generate a connection between the apparatus and the patient and device management platform needs to be undertaken less frequently.

In some embodiments the apparatus may be configured to store answers, and transmit answers to the patient and device management platform when a drying process is activated (either automatically or by the user).

In block 5816, the patient and device management platform takes an action based on the set of answers being processed. For example, such action can include identifying the patient or machine identifier (e.g., alphanumeric, barcode) associated with the patient answers, matching the patient or machine identifier associated with the patient answers with a patient or machine identifier (e.g., alphanumeric, barcode, or serial number) stored via or accessible to the patient and device management platform, locating a patient or machine profile (e.g., data structure, database record) based on the patient or machine identifier, writing the patient answers to the patient or machine profile, reading the patient or machine profile including the patient answers, and performing one or more analytic functions (e.g., plotting patient answers against time, predicting non-change or positive or negative change to patient health condition based on patient answers) on the patient or machine profile, as further described below. For example, the patient and device management platform may merge i.e. fuse enquiry data (e.g. answer data provided from the answers to the enquiry) and sensor data (e.g., sourced from the breathing assistance apparatus 5700), and such fusion can be actionable. For example, the patient and device management platform e.g. a server can be programmed to detect change to answers to queries over time and make that a data point, which can be actionable.

In some embodiments, the sensor data may be one or more patient parameters. For example, respiratory rate or oxygen saturation of the patient (as described in more detail elsewhere in the specification).

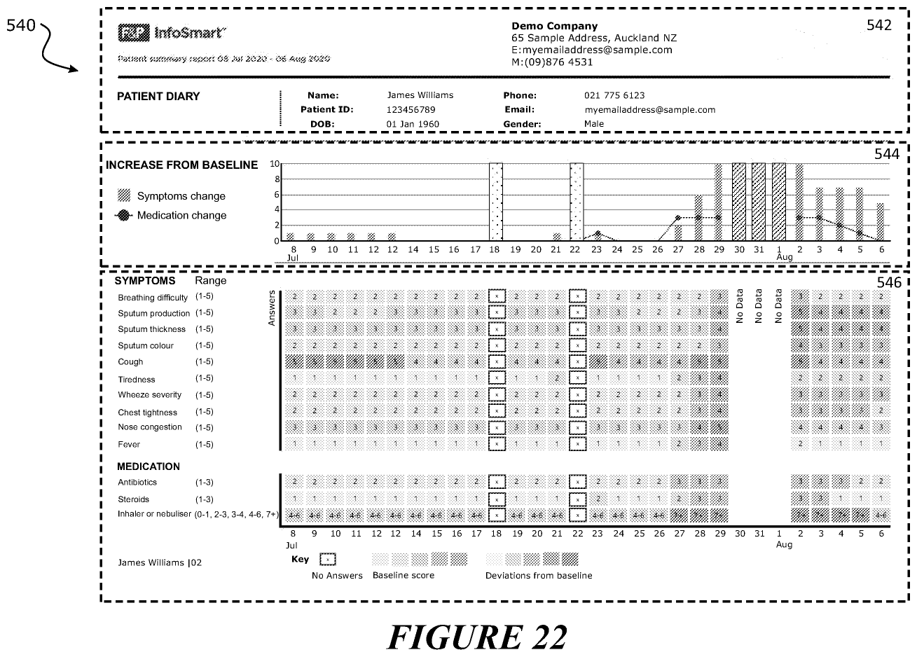

In block 5818, the action can include the server plotting or graphing the set of answers or processed answers relative to baseline data in one or more time-series charts, graphs or plots, as shown in FIGS. 22-26. The charts, graphs and/or plots can provide a graphical representation of the patient's health parameters and change in the patient's health parameters. The charts, graphs and/or plots can also indicate a patient health condition.

The charts, graphs, and/or plots may be part of a report (e.g. data report) presented to a user (for example a patient, caretaker, or healthcare provider).

In some embodiments the set of answers, and optionally a plurality of answers or sets of answers to a plurality of queries of a personal health enquiry (for example including historic health enquiries which may be stored in memory of the apparatus and/or the patient and device management platform) may be plotted, or graphed and/or form part of the report.

In some embodiments, the apparatus is configured to generate and/or display the report on the screen.

In some embodiments, the patient and device management platform is configured to generate the report, and the apparatus is configured to display the report on the screen of the apparatus.

In some embodiments, the patient and device management platform is configured to generate the report, and the apparatus is configured to provide the report to a healthcare provider.

In block 5820, the action can include the patient and device management platform e.g. server notifying non-patient (e.g., family member, doctor, caretaker) via a computing device (e.g. smartphone, tablet, wearable, workstation) in signal communication (e.g., wired, wireless, waveguide) with the patient and device management platform e.g. a data server. Such notice can include text, graphics, audio, video, or other forms of content. For example, such notice can be manifested via a mobile app, a dedicated software application, a browser-navigable portal, or other forms of software. The notice can be prompted or generated based on the set of answers satisfying or non-satisfying a predetermined threshold, as further described below. For example, the threshold can be satisfied based on a predetermined deviation from a baseline set by a doctor of the patient.

In some embodiments, the baseline may be set by the doctor (or other healthcare provider) based on an initial set of tests of the patient.

In block 5822, the action can include the patient and device management platform e.g. a data server notifying the patient (or caretaker in care proximity of patient) via a computing device (e.g. smartphone, tablet, wearable, workstation) in signal communication (e.g., wired, wireless, waveguide) with the patient and device management platform e.g. data server or the breathing assistance apparatus 5700 in signal communication with the data server via the network interface 5714. Such notice can be generated based on the set of answers and can include text, graphics, audio, video, or other forms of content. For example, such notice can be manifested via a mobile app, a dedicated software application, a browser-navigable portal, or other forms of software. For example, such notice can be output via the speaker 5708 or the display 5712. The notice can be prompted or generated based on the set of answers satisfying or non-satisfying a predetermined threshold, as further described below. For example, the threshold can be satisfied based on a predetermined deviation from a baseline set by a doctor of the patient. In some embodiments, when notice may be provided on the apparatus, or to a healthcare provider.

After the enquiry has been completed, the controller 5704 can control the display 5712 to present a screen (e.g., page) after the controller 5704 receives the set of answers, whether before, during, or after sending to the patient and device management platform e.g. data server. The screen presents a menu to control or activate the flow generator 5710 or the humidifier 5716 or to input an operational parameter of the flow generator 5710 or the humidifier 5716. Note that although the process 5800 is performed via the breathing assistance apparatus 5700 and the patient and device management platform, in certain situations, the process 5800 can be performed locally. For example, instead of sending the set of answers to the patient and device management platform, the breathing assistance apparatus 5700 (i.e. breathing assistance apparatus 10) can be programmed to perform similar processing locally and then take actions, as described herein. For example, the breathing assistance apparatus 5700 can perform blocks 5820 or 5822.

### 3.6 Determining Deviations in Answers Based on Preset Baselines

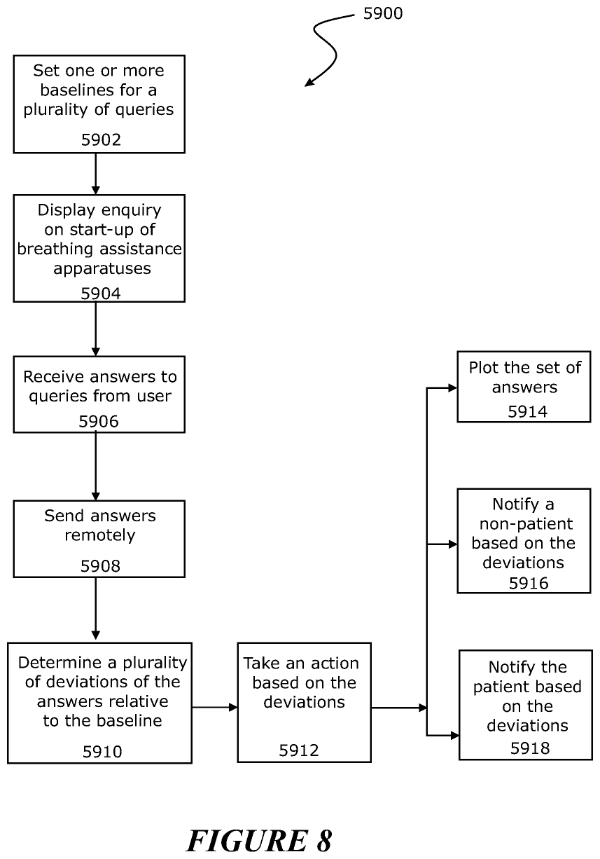

FIG. 8 is a flowchart of a process for determining deviations to answers of a personal health enquiry based on preset baselines. In particular, a process 5900 is performed via the breathing assistance apparatus 5700 and the server, as described above.

In block 5902, a user (e.g., doctor, nurse) operates a computing device (e.g., smartphone, tablet, wearable, workstation) in signal communication (e.g., wired, wireless, waveguide) with a patient and device management platform (e.g. a server web, application, database, cloud service). As such, the user accesses (e.g., browser session, dedicated software application session) a patient or machine profile via the server and sets a plurality of baselines for a plurality of queries of a personal health enquiry associated with the patient or machine identifier. For example, the patient and device management platform e.g. data server can present a browser-based user interface to the computing device operated by the user, where the user interface is programmed to receive a plurality of user inputs (e.g., alphanumerical, binary, or Boolean values entered or selected via textboxes, checkboxes, dropdown menus, radio buttons, sliders, dials) corresponding to the baselines as the baselines one-to-one correspond to the queries such that answers to the queries can be subsequently measured against the baselines, as set by the user.

In block 5904, the breathing assistance apparatus 5700 (e.g., controller 5704) displays the personal health enquiry, as shown in FIGS. 9 to 18, on the display 5712 via the controller 5704 to a patient (or caretaker or doctor) upon start-up of the breathing assistance apparatus 5700. This start-up can include booting of the breathing assistance apparatus 5700 (e.g., controller 5704) or activation of the breathing assistance apparatus 5700 (e.g., flow generator 5710, humidifier 5716) prior to use if the breathing assistance apparatus 5700 (e.g., controller 5704) is already booted and running. The enquiry comprises one or more queries that are presented onto the screen 212 i.e. display 5712.

In block 5906, the breathing assistance apparatus 5700 (e.g., controller 5704) receives a plurality of answers to the personal health enquiry from the patient. Such receipt can be performed via the patient touch selecting various user input elements (e.g., graphics, text, icons) displayed on the display 5712 via the controller 5704. The patient may also respond to the queries by performing a gesture or touching a portion of the display or pressing a portion of the display.

In block 5908, the breathing assistance apparatus 5700 (e.g., controller 5704) sends (e.g., wired, wireless, waveguide, encrypted, decrypted, unencrypted) the answers (e.g. answer data) via the network interface 5714 to the patient and device management platform (e.g. a server) remote from the breathing assistance apparatus 5700 such that the patient and device management platform can receive the answers and process the answers, as described below. The controller 5704 can request the network interface 5714 to send the answers (e.g., predetermined plurality of user touch inputs) to the server one-by-one after each of the answers is received via the controller 5704 (e.g., answer-send followed by another answer-send). The controller 5704 can request the network interface 5714 to send the answers (e.g., predetermined plurality of user touch inputs) to the patient and device management platform e.g. server after all of the answers (e.g., user touch inputs) are received via the controller 5714 (e.g., single send operation, single packet). The controller 5704 can request the network interface 5714 to send the answers (e.g., predetermined plurality of user touch inputs) to the patient and device management platform e.g. server on a group-basis after a group of the answers is received via the controller (e.g., send after every two, three, four, five, six, seven etc. answers). Note that the controller 5704 can request the network interface 5714 to send the answers with a patient or machine identifier in order to enable effective server-based identification of the answers and subsequent processing.

In block 5910, the patient and device management platform e.g. server receives the answers from the network interface 5714, identifies the patient or machine identifier (e.g. alphanumeric, barcode) associated with the patient answers, matches the patient or machine identifier associated with the patient answers with a patient or machine identifier (e.g. alphanumeric, barcode) stored via or accessible to the server, locates a patient or machine profile (e.g. data structure, database record) based on the patient or machine identifier, writes the patient answers to the patient or machine profile, retrieves the baselines that have been previously set, reads the patient or machine profile including the patient answers, performs a comparison between the baselines and the answers (e.g., baselines to answers or answers to baselines), and determines a plurality of deviations (e.g., binary value, Boolean value, alphanumeric value) of the answers relative to the baselines, if such deviations exist. Note that some of the deviations can include a degree of deviation (e.g., up by 5 points relative to baseline or down 10% relative to baseline).

Alternatively, the process of determining deviations relative to a baseline may be implemented and executed by the controller of the breathing assistance apparatus instead of the patient and device management platform.

In some embodiments, in block 5910, the patient and device management platform and/or the apparatus may determine deterioration of patient health.

The patient may be considered to be unstable if their health parameters are not stable.

Deterioration of patient health may be indicative of the patient being at risk of an onset of an exacerbation, or being at risk of an exacerbation.

The exacerbation may be a COPD exacerbation.

The deterioration of patient health of may be indicative of one other health issues for example a cold, hay fever, an allergic reaction etc.

The indication of a deterioration of patient health may allow for a health provider to act to mitigate further deterioration. Such action may help to avoid an undesirable outcome (e.g. hospitalisation).

The patient and device management platform and/or the apparatus may determine instability in patient health based on the answers to user queries, and one or more historic answers to the user queries.

The patient may be determined to be deteriorating if there is a worsening of at least one health parameter (optionally relative to a baseline).

The patient may be determined to be deteriorating if there is a worsening of two or more health parameters for at least two days (optionally relative to a baseline).

Additionally, or alternatively, the patient may be determined to be deteriorating if there is a worsening in one or more patient parameter and/or one or more patient parameter drops below a threshold and/or one or more patient parameter changes by more than a threshold (for example blood oxygen concentration or respiratory rate).

The patient may be determined to be deteriorating if there is a worsening in one or more patient parameters (for example blood oxygen concentration or respiratory rate) from a baseline.

For example, the patient may be determined to be deteriorating if SpO₂ gets worse or drops below a threshold of for example 85%.

For example, the patient may be determined to be deteriorating if respiratory rate increases by a threshold of for example 25%.

In some embodiments, the patient may be determined to be deteriorating if for at least two days there is both: a worsening of one or more health parameters (optionally from a baseline), and a worsening in one or more patient parameters (optionally from a baseline) and/or one or more patient parameters drops below a threshold and/or one or more patient parameters changes by more than a threshold.

For example, the SpO₂ and respiratory rate examples above may additionally require a worsening of at least one health parameter for the patient to be determined to be deteriorating.

In block 5912, the patient and device management platform (e.g. server) takes an action based on the deviations (or absence thereof) being processed. For example, the patient and device management platform can perform one or more analytic functions (e.g., plotting patient answers against time) as further described below.

In block 5914, the action can include the server plotting or graphing the set of answers or processed answer data relative to baseline data, as shown in FIGS. 22-26, such that the deviations are visually identifiable or visually distinct.

In block 5916, the action can include the patient and device management platform (e.g. server) notifying a non-patient (e.g., family member, doctor, caretaker) via a computing device (e.g. smartphone, tablet, wearable, workstation) in signal communication (e.g., wired, wireless, waveguide) with the server. Such notice can be generated based on the deviations (or absence thereof) and can include text, graphics, audio, video, or other forms of content. For example, such notice can be manifested via a mobile app, a dedicated software application, a browser-navigable portal, or other forms of software.

In block 5918, the action can include the patient and device management platform (e.g. server) notifying the patient (or caretaker in care proximity of patient) via a computing device (e.g. smartphone, tablet, wearable, workstation) in signal communication (e.g., wired, wireless, waveguide) with the server or via the breathing assistance apparatus 5700 in signal communication with the server via the network interface 5714. Such notice can be generated based on the deviations (or absence thereof) and can include text, graphics, audio, video, or other forms of content.

In some embodiments, the action can include notifying the patient and/or notifying a non-patient (e.g., family member, doctor, caretaker) based on a determination that the patient is deteriorating.

In situations where patient or caretaker notification is desired, then the network interface 5714 can receive a message (patient or caretaker notice) from the server, where the message is based on a predetermined plurality of user inputs (patient enquiry answers) previously sent to the server via the network interface 5714. For example, such notice can be manifested via a mobile app, a dedicated software application, a browser-navigable portal, or other forms of software. The message can include a video content for output via the display 5712. When the housing 100 houses the speaker 5708, then the message can include an audio content for output via the speaker 5708.

In situations where the breathing assistance apparatus 5700 has a plurality of network interfaces 5714 (e.g., Wi-Fi, Bluetooth), the breathing assistance apparatus 5700 can send a second message (e.g., patient or caretaker notice) to a computing device (e.g., smartphone, tablet, medical accessory) responsive to the first message (patient or caretaker notice from server responsive to patient enquiry answers). The second message can be sent by the second network interface (e.g., Bluetooth) and still be associated with the predetermined plurality of user inputs sent to the server via a first network interface (e.g., Wi-Fi). The second network interface (e.g., Bluetooth) can be local to the computing device, where the computing device is other than the server.

The message from the patient and device management platform can be informative of the patient health parameter worsening over a predetermined time period (e.g., at least two, three, four, five, six, seven days) as determined based on at least one of the predetermined plurality of user inputs. For example, the message can be informative of the patient health parameter worsening relative to a baseline, which may or may not be factory-set. For example, as explained herein, the doctor may be operating a computing device (e.g., phone, tablet, workstation) to set the baseline, where the computing device is in communication with the server, yet remote from the patient and device management platform e.g. server.

The message from the patient and device management platform may be a notification that the patient is deteriorating.

The steps of FIG. 8 have been described with reference to interactions between the breathing assistance apparatus and the patient and device management platform. However, in an alternative implementation steps described in blocks 5902, 5904, 5906, 5910, 5912 may be executed only by the breathing assistance apparatus 5700 (i.e. breathing assistance apparatus 10). For example, the processing in block 5910 may be performed by the controller of the breathing assistance apparatus and the options in block 5912 may be performed by the controller of the breathing assistance apparatus. For example, block 5918 may comprise presenting notifications to a patient upon the display of the breathing assistance apparatus.

In a further alternative implementation, the responses to the queries can be received at the controller of the device 5906. The queries may be transmitted to a user device (e.g. a tablet or a mobile phone) or the queries may be downloaded onto a USB and then transferred to a laptop or PC for further processing. The block 5910 may be executed by a user device or by the laptop or PC. Further, the functions described at block 5914 and 5918 may be executed by the user device or laptop or PC. The user device or laptop or PC may be in signal communication with a non-patient device e.g. a physician server or physician computing device. The notifications at block 5916 may be provided by the user device or laptop or PC to the non-patient device to notify a non-patient about the patient health condition.

### 3.7 User Interface Presenting Queries of Personal Health Enquiry

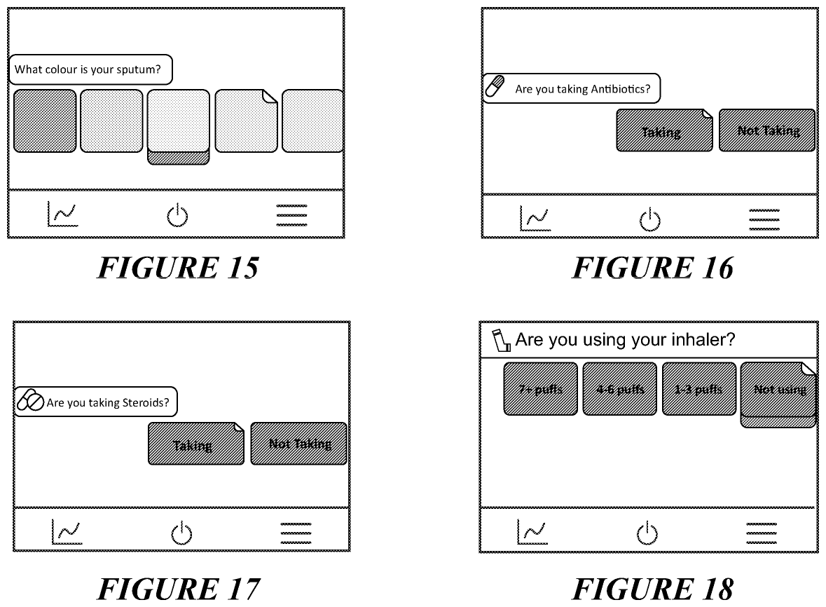

FIGS. 9 to 18 show a user interface presenting a personal health enquiry on a breathing assistance apparatus. The user interface includes a plurality of screens (e.g., pages) on which a plurality of queries and a plurality of potential answers are distributed. However, the user interface includes a single screen presenting the queries and the potential answers. Although FIGS. 9 to 18 show the user interface with a plurality of screens (e.g., pages) that are presented in an order, as shown in FIGS. 9 to 18, this order is illustrative and can vary. For example, the screen shown in FIG. 15 (or any other FIGS. 11 to 18) can be presented before the screen of FIG. 16 (or any other FIGS. 11 to 18) or after the screen of FIG. 18 (or any other FIGS. 11 to 18). The display 5712 displays the user interface with the screens of FIGS. 9 to 18, which are included in the personal health enquiry, as described herein. Further, FIGS. 9 to 18 can be embodied as a single screen that is vertically or horizontally touch scrollable. Alternatively, each query may be presented as a separate scrollable page.

FIG. 9 shows a booting screen. The booting screen can be a first screen that the patient sees when the patient turns on the breathing assistance apparatus, as described herein. The booting screen also shows a set of manufacturer and device identification information (e.g., text, graphics).

FIG. 10 shows an introductory screen after the booting screen. The introductory screen displays a hello message (or some other introductory or welcoming message). The hello message indicates that the enquiry has started. Note that the hello message is started at a midway of a left vertical plane of the display 5712 to case visibility, but this positioning can vary (e.g., non-midway, right vertical plane, spanning between left vertical plane and right vertical plane, top horizontal plane, display center).

FIG. 11 shows a general feeling screen that presents a query (e.g., request for user health information, health query) inquiring about a general feeling of the patient at a specific time of day. The query is alphanumeric, but can include pictorial content, whether additionally or alternatively. The query includes a concluding query mark, although this can be omitted (e.g., select one of following choices). The specific day or time of day is dynamic and changes based on when this query is displayed. This change can occur based on time/date tracked via the controller 5704 (e.g., internal clock). For example, a first set time period can correspond to morning, a second set time period can correspond to afternoon, and a third set time period can correspond to night. This time-of-day dynamic change functionality can be omitted (e.g., how are you feeling now). Regardless, this query is related to a general feeling parameter, as described herein, and an answer of ‘worse’ would suggest a worsening of the patient's health condition, whether this query is related to a baseline or not.

The general feeling screen presents a group of user input elements (e.g., rectangular graphics containing embedded or overlaid text) associated with the query and representing a group of potential answers to the query, one of which can be selected exclusive to others (although this can vary in certain situations where multiple answers can be non-exclusively input). As shown, a first input element that denotes a current position or a non-change state of the health parameter (e.g., usual), a second input element that denotes an improvement in the health parameter (e.g., better), and a third user input element that denotes a deterioration of the health parameter (e.g., worse). The group of user input elements is between 2 and 9, although lower or higher numbers of user input elements are possible. The group of user input elements is a group of text strings and a group of graphics, whether defining a single group of content (e.g., text integrally embedded within graphics) or a plurality of groups of content. Regardless, although these user input elements are visually identical to each other but for potential answer content, at least two user input elements in the group of user input elements can be visually distinct from each other. For example, such visual distinction can be present based on graphic background or foreground color, graphic background or foreground shade of color, graphic shape, graphic size, font type, font size, font color, font shade of color, font arrangement, or other graphic or font characteristics.

In one embodiment, the patient health enquiry may be configured to auto-populate or complete the enquiry data if the patient indicates they feel the same as a previous day or the last time they completed the enquiry. For example, in one such embodiment, the first query screen of the enquiry may be a comparative feeling question based on the most recent historic enquiry carried out by the patient. For example, in the case of daily health questionnaires being presented to the patient, the first query screen may simply ask “Do you feel any different to yesterday” or “Do you feel the same as yesterday?”, and the patient may be presented with binary answer options of ‘yes’ and ‘no’. If the patient's answer indicates they feel the same or no different than the prior day, then the health enquiry data may be automatically populated or completed with the stored answer data from the previous day or other relevant comparative time period (i.e. the previous answer data is copied over as the current questionnaire answer data).

The user interface can present the queries and the potential answers based on user scrolling via the touchscreen and the submitting the answers when the enquiry is completed.

FIG. 12 shows a sore throat screen that presents a query, similar to FIG. 15. However, unlike the query and the potential answers of FIG. 15, the query and the potential answers of FIG. 12 relate to a throat soreness parameter of the patient. The query of FIG. 12 comprises multiple possible answers each with an associated icon. The icons are colour coded relative to the patient condition associated with the answer and have faces with expressions related to the patient condition associated with the answer (as described in more detail below). If the patient's doctor sets a baseline of, for example, the middle (light green) answer, then a patient's selection of anything to the left of that answer (the yellow or the orange answer) would suggest a worsening of the patient's health condition. Note that the baseline may be visually distinct (e.g., green) relative to other potential answers.

At least one user input elements (e.g., leftmost or rightmost) of the group of graphics corresponds to an alphanumeric text content other than the query (e.g., extremely and not at all) and that alphanumeric content is positioned external (e.g., graphic tooltip, graphic label) to the at least one user input element (e.g., vertical or horizontal orientation).

The faces as illustrated in FIG. 12 may have a range of expressions corresponding to different indications of comfort. For example, the expressions of the faces may range from a sad or unhappy face (corresponding to a negative response to the query) to a smiling or happy face (corresponding to a positive response to the query). The expressions of the faces may have a neutral face (corresponding to a neutral response to the query).

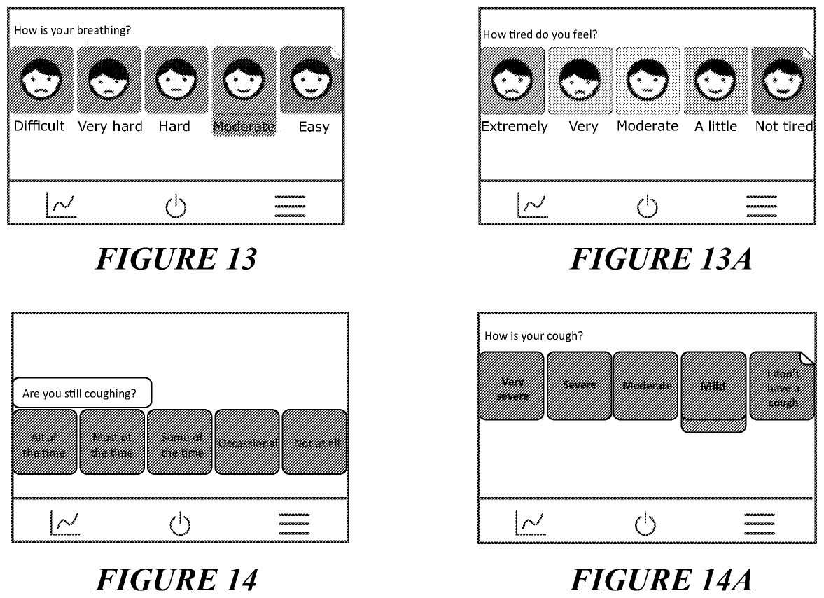

FIG. 13 shows a breathing screen that presents a query, similar to FIGS. 11 and 12. However, unlike the queries and the potential answers of FIGS. 11 and 12, the query and the potential answers of FIG. 13 relate to a breathing parameter of the patient.

FIG. 13A shows a tiredness screen that presents a query, similar to FIGS. 11-13. However, unlike the queries and potential answers of FIGS. 11-13, the query and the potential answers of FIG. 13A relate to how tired the patient feels.

FIG. 14 shows a coughing screen that presents a query similar to FIGS. 11 to 13A. However, unlike the queries and the potential answers of FIGS. 11 to 13A, the query and the potential answers of FIG. 14 relate to a coughing parameter of the patient.

FIG. 14A shows another coughing screen that presents a query similar to FIGS. 11-14. However, unlike the queries and potential answers of FIGS. 11-14, the query and potential answers of FIG. 14A related to the severity of the patient's coughing.

FIG. 15 shows a sputum color screen that presents a query similar to FIGS. 11 to 14A. However, unlike the queries and the potential answers of FIGS. 11 to 14A, the query and the potential answers of FIG. 15 relate to a query relating to a sputum color parameter of the patient.

FIG. 16 shows an antibiotic use screen that presents a query similar to FIGS. 11 to 15. However, unlike the queries and the potential answers of FIGS. 11 to 15, the query and the potential answers of FIG. 16 relate to a query relating to an antibiotic use parameter of the patient. If the patient has not been taking antibiotics, then a selection of the taking answer would suggest a worsening of the patient's health condition. Further, note that the potential answers are binary, although this can vary as needed. For example, FIG. 16 shows a pair of user input elements corresponding to the query relating to the antibiotic use parameter of the patient. The inputs from these user input elements are associated with a pair of data points that are mutually exclusive to each other (e.g., taking or not taking).

FIG. 17 shows a steroid use screen that presents a query similar to FIGS. 11 to 16. However, unlike the queries and the potential answers of FIGS. 11 to 16, the query and the potential answers of FIG. 17 relate to a query relating to a steroid use parameter of the patient. If the patient has not been taking steroids, then a selection of the taking answer would suggest a worsening of the patient's health condition.

FIG. 18 shows an inhaler use screen that presents a query similar to FIGS. 11 to 17. However, unlike the queries and the potential answers of FIGS. 11 to 17, the query and the potential answers of FIG. 18 relate to a query relating to an inhaler use parameter of the patient. If the patient's doctor sets a baseline of, for example, 1 to 3 times per day, then anything to the left of that answer would suggest a worsening of the patient's health condition.

It will be appreciated that a range of queries can be presented to the patient as part of the personal health enquiry. The queries may relate to a range of health parameters from any one or more categories, including a symptom category and a medication category for example. The queries may relate to any one or more of the following: patient breathing, patient tiredness, patient throat and muscles, where the patient has a temperature and/or shivers, patient infection, and any other subjective or objective health parameters in the symptom or medication categories or any other relevant health enquiry category or sub-category. Further examples of possible queries that may be presented to the user in or as part of the personal health enquiry are provided in the following paragraphs.

### Hospitalisation Queries

In some embodiments, the personal health enquiry may include a query asking about recent respiratory-related hospitalisation, or hospital admission or treatment, for example ‘Have you been to hospital?’ or a similar such query. The patient may be presented with the binary answer options indicative of ‘yes’ or ‘no’ for selection. If the patient answers ‘no’, then enquiry moves to the next health parameter query in the series. If the patient answer ‘yes’, indicating they have been in hospital or had recent hospital treatment, the enquiry may then present one or more additional follow-on queries relevant to the patient's recent hospitalisation. The one or more follow-on queries may include, for example, questions such as, but not limited to, ‘How long was your hospital stay?’, ‘What dates were you in hospital?’, ‘Are you able to go about your daily life?’, ‘How is your mobility?’, and the queries provide a range of response options for selection by the patient. In some configurations, one or more of these follow-on queries, which are triggered following the patient confirming recent hospitalisation, may be configured to be re-presented again in the personal health enquiry for a configurable period of time (e.g., for 2 weeks or 1 month following hospitalisation). The configurable period of time for presentation of these follow-on queries may be a default time period or may be adjusted or set by the patient's healthcare provider.

### Environment Queries

In some embodiments, the personal health enquiry may include one or more queries relating to the patient's environment and/or surroundings. Examples of such queries may include, but are not limited to, the following:
‘How is the weather?’—example answer options: ‘cold’, ‘hot’, ‘rainy’, ‘humid’ etc)
‘What kind of physical surroundings are you in?’—example answer options: ‘house’, ‘apartment’, ‘hospital’, ‘damp room’, ‘dry room’ etc
‘Is there flu in your community, family, workplace, and/or house?’—example answer options: ‘yes’ or ‘no’

The various one or more environmental questions may be accompanied by a range of selectable response options. As noted above, some queries may have binary response options indicating ‘yes’ or ‘no’, and others may provide a range of selectable response options, including categorical answers, rating or score answers, or any other suitable response options.

In some embodiments, the enquiry may have one or more primary environmental queries that have may have one or more associated follow-on or secondary queries that may be triggered for presenting to the patient depending or conditional on their answer to the primary environmental queries. In one example, a primary environmental query may be presented asking the patient ‘How is the weather?’, as above. If the patient answers ‘cold’ ‘or’ rainy, this may trigger one or more follow-on or secondary queries to be presented such as, but not limited to, ‘Do you think you feel worse because of this weather?’. In another example, a primary environmental query may be presented asking the patient ‘Is there flu in your community, family, workplace, and/or house?’, as above. If the patient answers indicating there is flu in their workplace or house, this may trigger one or more follow-on or secondary queriers to be presented such as, but not limited to, ‘Do you think you are experiencing any flu symptoms?’.

### Medication-Related Queries

In some embodiments, the personal health enquiry may be configured to automatically adjust or change or augment the queries depending or conditional on the processing and/or analysis of the enquiry data from the current and/or prior enquiries.

In one example configuration, the personal health enquiry may be adjusted to present one or more additional queries about the patient's perception of the efficacy or effectiveness of one or more medications being used by the patient, in response to a detection of an increase in medication use as indicated by the patient's previous answers relating to medication use.

For example, if the patient is using or has started using antibiotics, this information will be captured by the patient's responses or answers to queries presented about antibiotic use. The answer data relating to antibiotic use data may be processed relative to or based on one or more predetermined or configurable conditions, criteria, trend analyses, and/or thresholds (e.g., the criteria may be indicative of an increase or commencement of antibiotic use). If the criteria are met, the personal health enquiry may be automatically adjusted to include one or more additional follow-on queries about whether or not the patient feels that the antibiotics are helping their condition.

The above also applies to any other medication-related queries. For example, if the answer data for queries related to a specific medication indicate a significant change in use (e.g. an increase or decrease based on predetermined or configurable criteria or conditions), this may trigger the presentation of one or more additional follow-on questions relating to the patient's view on the effectiveness or efficacy of the medication for their condition or how they feel generally. In particular, the additional follow-on queries related to the effectiveness or efficacy of the medication are triggered conditionally or based upon analysis or processing of the patient's answer data relating to their use of the medication.

### Intended Use Queries

In some embodiments, the personal health enquiry may be configured to present queries to the patient about their intended use of the breathing assistance apparatus. The intended usage responses to such queries may be recorded in the enquiry data. The intended usage responses in the enquiry data may then be processed. In one configuration, one or more prompts, alerts, notifications and/or messages may be triggered for the patient and/or their healthcare provider based on the intended usage responses and optionally other enquiry data or other data relating to the patient or their therapy (e.g., prescription data, compliance data, and/or actual usage data). Additionally, or alternatively, one or more follow-on queries relating to the patient's usage of the apparatus may be presented to the patient in the enquiry based on the intended usage response and optionally other enquiry data or other patient data.

For example, at the beginning of a course of therapy treatment with the breathing assistance apparatus (e.g., in accordance with a prescription from a healthcare provider), the personal health enquiry may include one or more queries related to how often the patient intends to use the breathing assistance apparatus. The queries may relate to intended frequency of use (e.g., daily, twice daily, every 3 days etc), intended length of use in each therapy session (e.g., 3 hours per session, 8 hours per session etc), a combination (e.g. 8 hours per day cumulatively over one or more therapy sessions), and/or any other suitable intended use metric. The query or queries may be provided with a range of selectable response options or free-form text and/or numerical fields for the patient to enter their answer data. The patient's intended usage response may be more or less than their prescription for example.

In one configuration, the intended use answer data may be processed by the breathing assistance apparatus and/or data server (e.g., patient and device management platform), along with other data such as actual usage data recorded or stored in the breathing assistance apparatus and/or data server, and one or more notifications, prompts, messages, or alerts may be triggered if the data meets certain predetermined and/or configurable trigger criteria or conditions. For example, if the patient responds to the intended usage query indicating that they intend to use the breathing assistance apparatus for 8 hours per day, but the breathing assistance apparatus only detects usage for 3 hours per day as indicated by stored actual usage data, this may trigger a message, alert, notification or prompt to the patient and/or their healthcare provider that the patient's actual usage is below or significantly below their intended usage and/or the prescribed usage.

Additionally, or alternatively, the breathing assistance apparatus may trigger one or more additional intended usage queries to be presented to the patient in the personal health enquiry if their intended usage data and actual usage data meet certain predetermined and/or configurable trigger criteria or conditions. For example, if the patient's actual usage is identified or determined to be below or significantly below their intended usage in accordance with the trigger criteria or conditions, the system may be configured to present one or more additional queries to the patient in the personal health enquiry seeking information or data on why the patient is not using the breathing assistance apparatus as much as they intended.

### Questionnaire Feedback Queries

In some embodiments, the personal health enquiry may be configured to present one or more queries to the patient seeking their feedback on the usefulness of the queries. By way of example, the one or more feedback queries may include, but are not limited to, queries such as the following:
‘Are there any other questions you think should be asked?’
‘Have you been experiencing any symptoms that have not been captured by the questionnaire?’
‘Have you made any changes to your habits (e.g. exercise or food and drink intake) that have not been captured by the questionnaire?’

The above feedback queries may be presented with free-form text response fields for the patient to enter their feedback and/or predetermined answer options, depending on the nature of the feedback question.

### Patient Comfort Queries

In some embodiments, the personal health enquiry may be configured to present one or more queries related to the patient's comfort during respiratory therapy with the breathing assistance apparatus. For example, the patient may be presented with a query or queries such as, but not limited to, ‘Are you finding the therapy comfortable?’. The patient may be presented with a range of different selectable answer options. In some configurations, the answer options may be binary answers such as ‘yes’ and ‘no’, and in other configurations, the patient may be able to select their comfort level answer on a scale or from a range of comfort levels. This patient comfort answer data is included in the enquiry data and may be later processed for display or rendering in one or more reports or charts, similar to the other enquiry data.

### Other Example Queries

It will be appreciated that a range of different queries may be presented in the patient health enquiry. Some queries may be presented in every enquiry, while others may be configured to presented or included in the enquiry at a specified lower frequency based on time or the number of enquires (e.g., the query may be presented in the enquiry every 2 weeks or monthly, or every 5th enquiry).

Below are some further examples of queries that may be presented to a user, and possible answer options that may be presented for selection:
Since your last [biweekly/monthly] checkup, did you need any unscheduled health visit related to your COPD? Please select all relevant options. Answer options: Prefer not to answer, None, GP, A&E visit, In-hospital stay.
How would you rate your health? Answer options: Prefer not to answer, Poor, Fair, Good, Very Good, Excellent.
Since your last [biweekly/monthly] checkup, has your health affected your ability to perform physical tasks? Answer options: Prefer not to answer, Not at all, A little bit, Moderately, Quite a bit, Extremely.
Since your last [biweekly/monthly] checkup, how much has your condition affected your mental wellbeing? Answer options: Prefer not to answer, Not at all, A little bit, Moderately, Quite a bit, Extremely.
Since your last [biweekly/monthly] checkup, has your health affected your social activities? Answer options: Prefer not to answer, Not at all, A little bit, Moderately, Quite a bit, Extremely.
Please rate your experience with therapy. Answer options: Prefer not to answer, Poor, Fair, Good, Very Good, Excellent.

### Queries Presented Based on Prior Answers

As discussed above, there are various examples in which the answers to queries may then trigger the presentation of one or more additional or follow-on queries in the patient health enquiry. For example, the presentation of some queries may be a function of or conditional upon the patient's answers to one or more prior queries in the questionnaire.

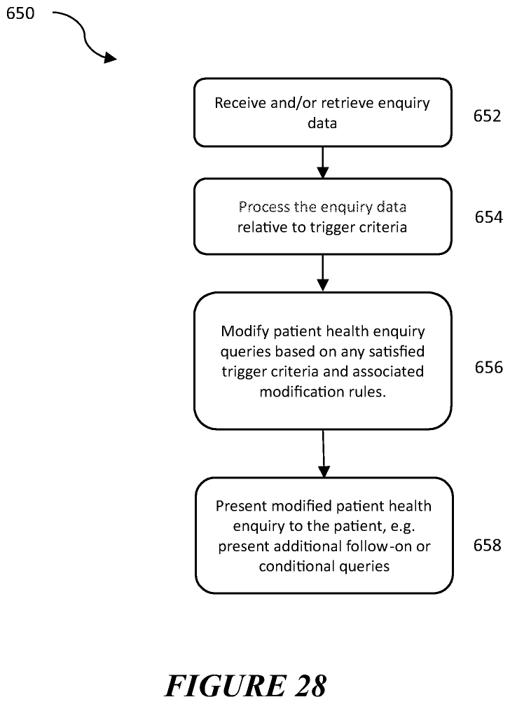

One example configuration of the above process will be explained with reference to FIG. 28. The example process 650 in FIG. 28 initiates with the processor (e.g. a process of the data server and/or breathing assistance apparatus) receiving or retrieving the incoming enquiry data which includes the patient's answers to the presented queries, as shown at step 652. The received enquiry data is then processed relative to one or more trigger criteria as shown at step 654. If the patient's answers in the enquiry data satisfy any one or more trigger criteria, then the controller is configured to modify the patient health enquiry presented to the patient in accordance with stored respective modification rules associated with the trigger criteria, as shown at step 656. For example, a specific main or primary query in the enquiry may have trigger criteria that triggers one or more additional follow-on secondary queries to be presented to the patient depending on their answer to the first primary query. In this example, the trigger criteria specifies the type of answer or answers that will trigger the follow-on or additional queries to be presented, and the associated modification rules define the specific follow-on or additional queries that are inserted into the enquiry for presentation if the criteria are satisfied. Once any modification rules have been implemented, the modified patient health enquiry is presented to the patient, as shown at step 658. This last step may involve the enquiry dynamically modifying the next query or queries presented, based on or as a function of the answers to the prior one or more queries, i.e. the content (nature and type of queries presented) of the enquiry may dynamically change as it progresses, depending on the patient answers.

### Battery Mode

If the breathing assistance apparatus is operating on battery power in a battery mode, the personal health enquiry may still be presented on the display of the breathing assistance apparatus and the patient responses captured and stored as answer data.

### 3.8 Patient Baseline Data

As described above, a user of the breathing assistance apparatus is provided an enquiry, formed of one or more queries, on an integrated touchscreen of a breathing assistance apparatus. The enquiry is received on one or more touch screens, through which the user also provides their response. The responses (answer data) are then processed, and one or more reports comprising charts, graphs, plots or other graphical formats of the data are then generated for display to visually illustrate the health parameters recorded by the patient.

As shown in FIG. 8, in some embodiments a baseline 5902 of health status for a patient is first established. For example, this baseline status of the patient's health parameters may be established independently by a healthcare provider, or established by the patient answering one or more queries of an enquiry from the apparatus. In such an embodiment where a baseline status of a patient's heath parameters is established, a healthcare provider (and/or the patient) may be able to determine a change from baseline, whether that be an improvement in one or more health parameters or a worsening in one or more health parameters. In one embodiment the specific baseline for a particular patient may be stored locally on the breathing assistance apparatus or may be accessed from a remote device (e.g. a server). In one configuration, the baseline may be represented by baseline data stored against the patient's profile in the patient and device management platform. This baseline data may then be accessed and/or retrieved by the breathing assistance apparatus from the patient and device management platform.

Each health parameter may comprise an associated baseline.

The baseline for a health parameter may be dynamically and/or automatically adjusted or updated (for example by the apparatus and/or patient and device management system).

The baseline may be updated based on the answers to one or more queries as part of the health enquiry over a period of time.

In some embodiments the baseline for a health parameter may be updated based on a period of consistent answers to a query or enquiry by patient. In one example configuration, the baseline for a health parameter may be updated if the answers to the query or queries related to the health parameter are all the same or within a predetermined deviation threshold of the baseline. In another example configuration, the baseline for a health parameter or parameters may be automatically updated if their respective answers (e.g. answer data for the queries related to the health parameter) indicate a consistent new value for a predetermined or configurable period of time and/or number of completed enquiries (e.g., the baseline for a health parameter may be updated to the latest answer value if that answer has been consistent for a predetermined number of consecutively completed questionnaires and/or for a predetermined number of days, weeks, and/or time intervals).

The baseline may be updated if, the answers to the query or queries related to the health parameter, indicate a different baseline. The baseline may be updated to the different baseline if the answers to the query or queries related to the health parameter indicate a different baseline over a predetermined time, or predetermined number of responses to the health enquiry.

In some embodiments, the baseline may be iterated towards the different baseline.

For example, if the patient answers that they have an occasional cough (i.e. FIG. 14) ten times in a row, then the baseline associated with this patient parameter may be updated to be an occasional cough.

In some embodiments, before the baseline is automatically updated the patient may be prompted as to whether they would like the baseline to be updated. For example, in some configurations, any proposed automatic adjustment or updates to the baseline data may require approval from the patient.

In some embodiments, before the baseline is automatically updated the healthcare provider (for example a doctor) may be prompted as to whether they would like the baseline to be updated. For example, in some configurations, any proposed automatic adjustments or updates to the baseline data may require approval of the healthcare provider before being updated.

In some embodiments, the baseline data may be updated periodically (for example once every 6 months). For example, new or fresh baseline data for a patient may be obtained by a healthcare provider, and/or may be established from the patient completing a patient health enquiry, and/or may be determined based on analysis of previous historical enquiry data related to previously completed patient health enquiries.

In some embodiments, the healthcare provider may be able to select a specific set of enquiry data relating to a specific completed patient health enquiry to be used as the new patient baseline data. In one configuration, the healthcare provider may be able to select the desired set of enquiry data via interaction with one or more patient data reports or graphical data extracts (e.g., time-series charts, time-series graphs, heatmaps, or the like, examples of which are further described later). In one example, the healthcare provider may select or designate a specific column of the patient's heatmap (see FIGS. 22-24 explained later) as the patient's new baseline data. The healthcare provider may interact with the GUI displaying the heatmap, and interact with one or more GUI elements to designate or select a desired column of data from the patient's heatmap as the new patient baseline data. The enquiry data from the selected column of the heatmap relates to the patient's questionnaire answers for a specific day, week or time interval. The healthcare provider may select or designate the enquiry data for a specific time interval as being representative of the patient's current baseline. The enquiry data associated with the completed questionnaire from the selected column or time interval (e.g. specific day or week along the time axis) of the heatmap is then loaded as the patient's new baseline data, i.e., the baseline associated with each health parameter query is then reset to be equal to its respective answer in the selected column of the heatmap. It will be appreciated that the healthcare provider could also operate or instruct the system to reset a patient's baseline data to correspond to a specific set of enquiry data from a specific day, week or other time interval via other input commands or interaction with a control or configuration interface of the patient and device management platform.

A healthcare provider may update or adjust the baseline data via interaction with the breathing assistance apparatus and/or remotely via interaction with the patient and device management platform.

In some embodiments, a temporary event-based baseline may be used for a patient, conditional on specific events, and may remain the baseline for a configurable or predetermined time-period before reverting to the patient's normal or typical baseline data. For example, a healthcare provider may be able to configure the system to load or designate a ‘post-hospital’ baseline for a patient that applies for a specific time period (e.g., a specific number of days or weeks) after the patient leaves or is discharged from hospital following an exacerbation event. Once the specific time period lapses, the baseline data for the patient may revert or change back to the patient's usual or normal baseline data.

In some embodiments, the system (e.g., breathing assistance apparatus and/or the patient and device management platform) may process the enquiry data and detect or identify that the patient's answers (e.g., answer data) for one or more queries in the patient health enquiry have stabilised according to predetermined or configurable criteria or conditions. In one example, the answer data may be considered to be stable or to have stabilised based on analysis of the deviation of the answer data relative to a moving average. If the deviation from the moving average is detected as being below a threshold, the answer data may be determined as being stable, for example. In one configuration, the system may be configured to trigger an alert, notification, or message to be sent or pushed to the healthcare provider prompting or asking the healthcare provider about whether they wish to update the patient's baseline data (or portions of the baseline data) to the latest enquiry data, if a certain number of answers (e.g., one, some, or all) have been detected or identified as being stable. The alert, notification, or message may be sent or pushed to an electronic device (e.g., smartphone, tablet, wearable, computer or the like) of the healthcare provider and/or provided on the user interface, portal or dashboard of the patient and device management platform. In response to the notification, the healthcare provider may then instruct the system to update some or all of the baseline data to the identified stabilised enquiry data.

In some embodiments, the data reports generated by the system may plot a time-series chart or graph displaying the baseline data over time. For example, one or more graphs may be generated that plot the change in the baseline data for one or more health parameters over time to illustrate long-term changes or trends in the patient's health. These baseline time-series charts may be plotted separately as their own charts, or may be integrated into the other enquiry data time-series charts (e.g., the time-series graphs and/or heatmaps described later with respect to FIGS. 22-26).

### 3.9 System Architecture

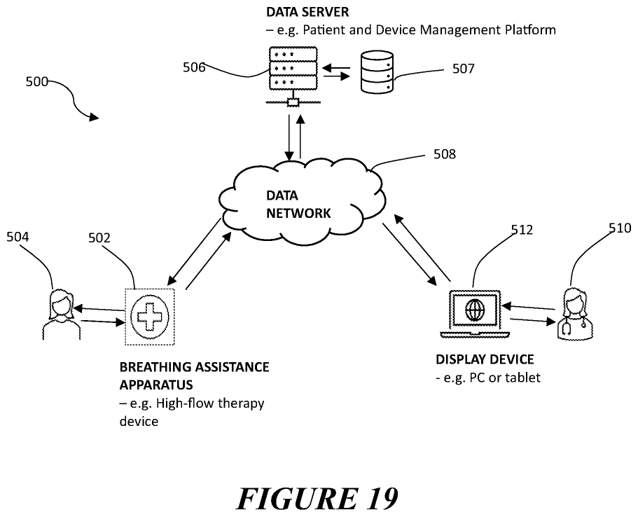

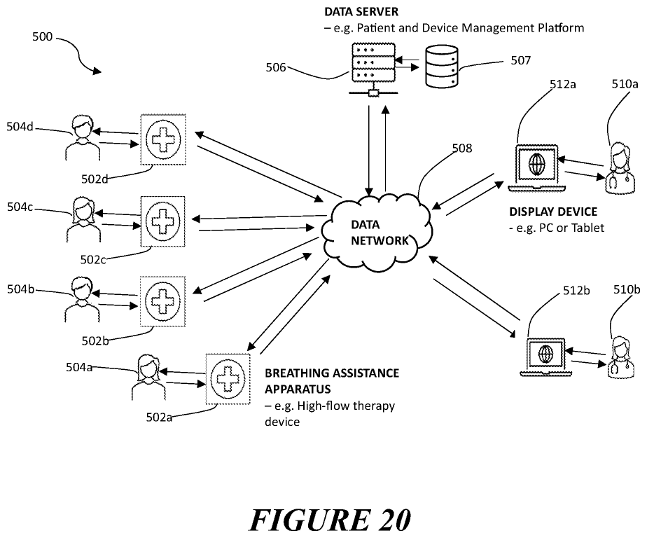

Referring to FIGS. 19 and 20, an example configuration and/or architecture of the overall system 500 for implementing the capture, transmission, processing, storage, retrieval and/or display data relating to the personal health enquiry described above will be explained in further detail. It will be appreciated that any one or more of the features, aspects, and alternatives of the system and process described above may apply to this example configuration.

In this embodiment, the system 500 comprises one or more breathing assistance apparatuses 502 (herein also referred to as ‘respiratory therapy device’). The breathing assistance apparatus 502 may be the breathing assistance apparatus 10 or 5700 described above for example. The breathing assistance apparatus 502 is operable to delivery respiratory therapy to a patient or user 504 as previously described.

The breathing assistance apparatus 502 is operable to present the personal health enquiry to the patient 504 as described. The personal health enquiry may comprise a series of queries as previously described. The user input to the queries via the user interface of the breathing assistance apparatus 502 is captured as answer data on the apparatus. Additionally, if the entire enquiry or specific queries within the enquiry are skipped by the user, such data is captured as skipped data.

The breathing assistance apparatus 502 is in data communication with a data server 506 over a data network 508 or data link as previously described. The breathing assistance apparatus 502 is configured to transmit or send the captured answer data and/or skipped data for each personal health enquiry to the data server 506 for storage and processing. The answer data and/or skipped data can be collectively referred to as the enquiry data. The phrases ‘data server’ and ‘patient and device management platform’ may be used interchangeably in this disclosure, unless the context suggests otherwise.

The data server 506 may be a cloud service or platform for example, or any other suitable data processing server, service or platform. The data server 506 may be a subscription-based 3^{rd} party platform or may be a proprietary system, or a combination. The data server 506 may perform, provide or host the functions of the patient and device management platform previously described. In some configurations, the data server comprises at least one processor or processor device, and memory.

The data server 506 is configured to retrieve, access and/or store baseline data representing the baseline health condition of the patient in regard to one or more health parameters corresponding to the queries in the patient health enquiry. As previously discussed, the initial baseline data may be provided from the patient via the breathing assistance apparatus or from the healthcare provider or may be preset or preconfigured baseline data. The data server may dynamically or periodically retrieve or receive updated baseline data, or may be configured to update and/or adjust the baseline data itself over time during processing of the incoming enquiry data based on automatic updating rules or algorithms, either periodically, on demand, or upon request.

In this embodiment, the data server 506 receives or retrieves the captured raw enquiry data for each personal health enquiry carried out or a representative set of enquiry data for the configurable time-interval of interest (e.g. daily or every 3 days, weekly, or any other suitable time interval frequency). The enquiry data may comprise identification information or data relating to either the breathing assistance apparatus and/or patient, so that the data sever can store or link the raw enquiry data to the profile of the relevant patient in a database 507 or other electronic storage of or linked to the data server. The enquiry data may also comprise time-stamp or time data representing or indicative of the time interval (e.g. date and/or time) in which the enquiry data was captured.

In this embodiment, the data server 506 is configured to process the enquiry data and associated baseline data for a patient to generate one or more data reports representing the personal health of the patient as represented by the enquiry data. In one configuration, the data report or reports comprise one or more time-series charts, e.g., graphs or plots, of the enquiry data over a series of time-intervals over a specific time period or a rolling window of enquiry data for the patient.

The data server 506 may be configured to process the enquiry data in real-time to generate the one or more data reports dynamically on request. Additionally, or alternatively, the data sever 506 may pre-process the enquiry data, as it arrives, into a processed form that is ready to render into one or more data reports for display or into a static or updated pre-compiled data report ready for retrieval and display.

In this embodiment, the one or more data reports relating to the patient may be accessed or retrieved by a healthcare provider 510 via a display device 512 that is in data communication with the patient and device management platform provided on the data server 506. The healthcare provider 510 may access that data report via any electronic device with a display screen (e.g. PC, tablet, smartphone, wearable or the like) and which has data access to the data server 506. By way of example, the display device 512 may access the patient and device management platform via a web browser or website interface, or via a software application program running on the display device, a user-access portal or interface, or via any suitable host-client architecture configuration. It will be appreciated that in some configurations a patient 504 may also access for display their own data reports via the GUI of the breathing assistance apparatus and/or any other suitable electronic device with a display screen and data communication with the data server 506.

The display device 512 of the healthcare provider may retrieve or access the data reports generated (pre-compiled or real-time generated) by the patient and device management platform 506 for the patient 504 for display or may retrieve or access raw data or pre-processed data representing the data report for further local processing into the data report for display.

Referring to FIG. 20, the overall system 500 will be explained further in the context of a system for managing the personal health enquiry data for multiple different patients. Like numerals represent like components. As shown, in some embodiments, the patient and device management platform 506 is configured as a data server for multiple patients 502a-502d, each operating their own breathing assistance apparatus 504a-504d. The patient and device management platform 506 may be configured to serve any number of individual patients. Likewise, the patient and device management platform 506 may be accessed by multiple different healthcare providers 510a,510b via their respective display devices 512a,512b. It will be appreciated that the number of different healthcare providers accessing the patient and device management platform may vary as desired.

By way of example, each different healthcare provider 512a,512b may have a group of one or more patients they are monitoring and/or treating, and may have access or permission to access or request the records and/or data reports associated with each of their respective patients via the patient and device management platform 506. In one configuration, the data reports may be generated in real-time or dynamically by the patient and device management platform 506 in response to a data request from a display device 512 operated by the healthcare provider.

In some configurations, the patient's breathing assistance apparatus 502 may be in data communication with one or more other local electronic devices, whether portable or non-portable. For example, the breathing assistance apparatus 502 may be data linked or in data communication (e.g., over Bluetooth, or Wifi, or any other suitable data communication protocol) with the patient's smartphone, tablet, wearable, and/or Personal Computer (PC). The linked electronic device may itself also be in data communication with the data server 506 over the data network 508. In some embodiments, the patient's linked electronic device (e.g., smartphone, tablet and/or PC) may be configured to receive or retrieve the enquiry data captured by the breathing assistance apparatus. The linked electronic device may be configured to process the enquiry data (along with stored or retrieved baseline data) either partially or entirely (as per the processing described in the data server), and may then present the processed data (e.g., data reports and/or time-series charts) to the patient on a display screen of the electronic device. Additionally or alternatively, the processed data (e.g., data reports and/or time-series charts) on the linked electronic device may be sent back to the breathing assistance apparatus 502 for display on the user interface (e.g., touchscreen display).

Additionally or alternatively, the linked electronic device may be configured to relay or send on the raw enquiry data and/or partially processed data and/or fully processed data to the data server 506 over the data network, so that the data server 506 can further process the data, store the data, and/or enable access to the data or processed data (e.g. data reports and/or time-series graphs) for review (e.g. by the healthcare provider on their display device 512).

Additionally or alternatively, the linked electronic device may be configured to receive processed data (data reports and/or time-series charts) from the data server 506. The processed data may be requested by the linked electronic device or pushed to the electronic device automatically, periodically, or based on adhoc or manual triggers. The processed data received by the linked electronic device may be presented or displayed on a display of the linked electronic device for viewing or interaction, and/or may be relayed or sent on to the breathing assistance apparatus 502 for display and/or interaction on the touchscreen display of the apparatus.

In some configurations, the patient's linked electronic device may be in data communication with the breathing assistance apparatus 502, data server 506, and the healthcare providers display device 512, either directly or via the data network 508.

In some configurations, the patient's linked electronic device (e.g., smartphone, tablet, wearable, computer or the like) may be configured or operable to present the patient health enquiry to the patient or user in a similar manner to the breathing assistance apparatus. In this configuration, the patient can complete the patient health enquiry via their linked electronic device, which may receive, store, process and/or transmit the enquiry data to the breathing assistance apparatus and/or data server for further processing. The digital patient health enquiry may be presented on a display of the linked electronic device via a dedicated software application program or via an interface such as a website or browser interface. In some configurations, the software application running on the linked electronic device (e.g., a smartphone application for example) may transmit the patient's enquiry data to the breathing assistance apparatus, which then relays the enquiry data onto the data server for processing. In other configurations, the linked electronic device may send the patient's enquiry data directly to the data server for processing.

3.10 Processing of Enquiry Data into Data Reports

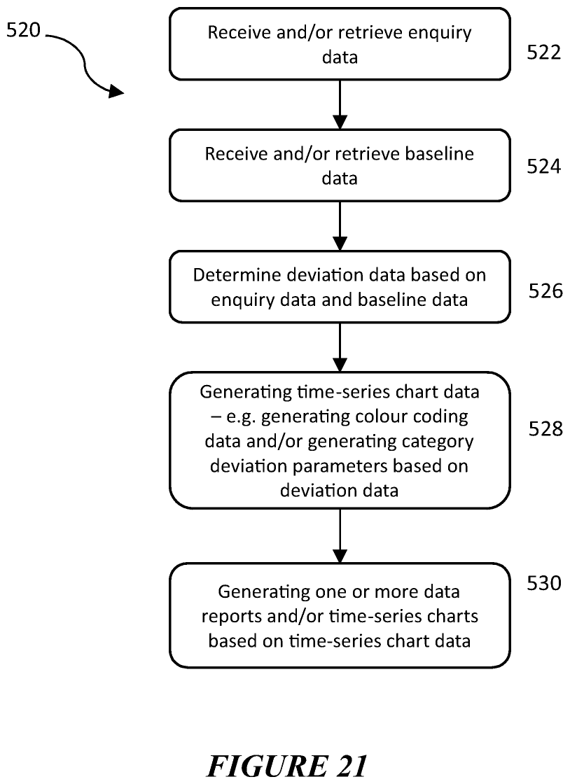

Referring to FIG. 21, an embodiment of the data processing flow 520 carried out by the system 500 for generating a patient data report comprising one or more time-series charts will be described. In this example embodiment, the data processing flow 520 is implemented in the patent and device management platform (e.g. data server 506), although it will be appreciated that the data processing flow, or one or more stages of it, may also be carried out locally at the breathing assistance apparatus 502 or display device 512 in alternative configurations. It will be appreciated that one or more of the steps or stages of the data process flow may occur in a different order or in parallel, in alternative configurations.

The data processing flow 520 may start in response to a data request and/or automatically in response to new incoming enquiry data. For example, in one mode, the data processing flow 520 executes dynamically in real-time upon request for a data report or data access by a display device 512 operated by a healthcare provider 510. In another mode, the data processing flow 520 automatically execute upon receiving fresh incoming enquiry data, so that updated data reports or processed data for such reports are updated and synchronised to the latest incoming enquiry data, ready for retrieval and display at a later time.

Upon execution, the data process flow 520 commences by receiving and/or retrieving enquiry data at step 522 for a patient. The enquiry data comprises enquiry data associated with a series of time intervals over a time period. In one configuration, the time period may extend from the latest or most recent enquiry time interval back to a configurable or preset earlier time interval. In other configurations, the time period may be configurable via configurable start and end time intervals (e.g. start date and end date).

For the purpose of this explanation, the time interval will be daily, and the time period extends over a calendar month of data. However, it will be appreciated that the data process flow can be applied to any configurable time interval frequency and time period (e.g., weekly over a number of weeks or months). For example, the time-interval frequency and time period settings may be configured or selected by the healthcare provider depending on their desired viewing preferences as to the time period of data to view and/or the granularity and/or resolution of data to view.

The data server 506 may receive or retrieve the enquiry data for the patient from the database 507 or electronic data storage or of associated with the data server. In this example, each set of enquiry data for a time interval (e.g. day) in the time-series may comprise the answer data comprising the answers to each of the queries in the enquiry and/or skipped data identifying any one or more queries that were skipped by the patient.

In some instances, the skipped data may indicate the entire enquiry (i.e. all queries were skipped by the patient). As mentioned, each day of enquiry data also comprises a time-stamp identifying the time interval it was captured. In this example, the time-stamp may be the date and/or time the enquiry data was captured. The enquiry data also comprises ID information that is sufficient to directly or indirectly link or associate the data with the patient (e.g. a patient ID, or breathing assistance apparatus ID or serial number).

In this embodiment, the personal health enquiry comprises a series of queries that are categorised or grouped into one or more categories of health parameters. In one configuration, the queries are grouped into a symptom category relating to the patient's symptoms, and a medication category relating to the medication the patient is taking. In this configuration, the answer data may be delineated into symptom data which is the answer data relating to the symptom category queries, and medication data which is the answer data relating to the medication category queries.

By way of example, in one embodiment, the symptom category queries may relate, but are not limited to, any one or more of the following health parameters: breathing difficulty, sputum production, sputum thickness, sputum colour, cough, tiredness, wheeze severity, chest tightness, nose congestion, and fever.

By way of example, in one embodiment, the medication category queries may relate, but are not limited to, any one or more of the following health parameters: antibiotics, steroids, and inhaler or nebuliser usage.

Once the enquiry data for the patient is received, the data process flow 520 then proceeds to receive or retrieve the baseline data for the patient, as shown at step 524. The baseline data for the patient may be stored in the database 507 or other electronic data storage of or accessible to the data server 506. The baseline data for each patient may comprise a baseline value or score or answer for each query in the personal health enquiry. As discussed above, the baseline data for each patient may be customised to the patient's baseline health condition. The baseline data may be periodically or continually updated or refreshed based on incoming enquiry data, as previously discussed.

The data process flow 520 then processes the enquiry data relative to the baseline data to determine deviation data, as shown at step 526. The deviation data may be indicative of any deviation of the enquiry data from the baseline data, for each time-interval. The deviation data may be on a query-by-query basis, and/or category basis. The deviation data may indicate a worsening, no change, or an improvement in the health parameter(s) and/or overall health condition of the patient, based on comparing the answer data to the baseline data at a query and/or category level.

In this example embodiment, each query presented to the patient provides a number or range of answers that can be selected, as previously described. The answer data to each query either directly represents or corresponds to a score or rating on a respective scale or answer scale, or is configured to be mapped or transformed to a corresponding score or rating on a scale. The scale comprises a series of scores or ratings ranging from a desirable score at one end (e.g. indicative of a ‘good’ health condition) to a non-desirable score at the other end (indicative of a ‘bad’ health condition). The scores or ratings scale may be numeric, or categorical, or may be a Likert-type scale, depending on the nature of the query and metric for assessing. The scales may be linear, progressive, logarithm, or arbitrary. The baseline data is provided against corresponding scales for each query. In this embodiment, the data process flow 520 is configured to determine the deviation data based on comparing the answer data relative to the baseline data with reference to the scale of each query. In one configuration, the deviation data may represent a compressed version or representation of the enquiry data relative to the baseline data for each patient.

After determining or calculating deviation data, the data process flow proceeds to generate time-series chart data based on the enquiry data, baseline data, and/or deviation data, as shown at step 528. The data process flow may generate one or more different formats of time-series chart data depending on the nature and type of time-series charts to be generated for display. In one configuration, the time-series chart data comprise a coded or processed data set of the enquiry data, baseline data, and/or deviation data. In one configuration, the time-series chart data is in the form of metadata that is associated or tagged or coded to the enquiry data, baseline data, and/or deviation data.

In one configuration, the data process flow 528 is configured to colour-code the enquiry data based on the deviation data. The colour-coding data may represent or form part of the time-series chart data. The colour-coding is selected according to a baseline colour spectrum and one or more deviation colour spectrums. In one configuration, there is a ‘worsening’ deviation colour spectrum indicative of a worsening health parameter relative to the baseline, and an ‘improvement’ deviation colour spectrum indicative of an improving health parameter relative to the baseline. The colour coding of the enquiry data selects a distinct colour spectrum for applying to the answer data in a colour-coded time-series plot, as will be explained in further detail below.

In another configuration, the data process flow 528 is configured to generate category deviation parameters that represent or are a function of the total number of health parameters in each category that have deviated (worsened or improved). In one form, there is an ‘improvement’ category deviation parameter representing the total number of health parameters in the category that have improved relative to the baseline, and a ‘worsening’ category deviation parameter representing the total number of health parameters in the category that have worsened relative to the baseline. The category deviation parameters may represent a data set for each time-interval of enquiry data. The category deviation parameters may represent or form part of the time-series chart data.

The time-series chart data may be stored in the database against the patient profile as a distinct data set or as metadata or related data to the enquiry data, baseline data, and/or deviation data. The time-series chart data can be retrieved or accessed at any point for the generation or rendering of one or more data reports, including the generating or rendering of one or more time-series charts based at least partly on the time-series chart data.

The data processing flow 520 includes generating one or more data reporting and/or one or more time-series charts of the enquiry data based at least partly on the time-series chart data, as shown at 530. These data reports and/or time-series charts may be pre-compiled in advance of retrieval or may be generated dynamically in real-time upon request, for example. Examples of the data reports and/or time-series charts will be explained in further detail below.

### 3.11 Data Reports Comprising a Plurality of Time-Series Charts

FIG. 22 shows an example of one form or format or layout of a data report 540 that can be generated or rendered based at least partly on the processed enquiry data, baseline data, and/or deviation data. In this example, the data report comprises a plurality of time-series charts, although it will be appreciated that the data report may comprise a single time-series chart in alternative configurations.

In this example data report 540, a patient information section if provided as indicated in the dotted area designated 542. This patient information includes patient data such as name, date of birth, contact details, patient ID data, or any other desired patient information.

A first time-series chart in the form of a colour-coded time-series plot is provided or rendered in the section indicated in the dotted area designated 546. In this embodiment, the colour-coded time-series plot is configured to depict the daily enquiry data over a time period (e.g a month) and each answer is displayed according to a colour spectrum that depends or is a function of any deviation from the baseline as determined or according to the colour coding data previously explained. The colour coding spectrums represent or convey both the general desirability of the answer on the scale, and any deviation relative to the baseline.

As previously described, the time interval between the patient health enquiry being presented to capture new enquiry data may be configured by a healthcare provider. In the embodiments described below, the time interval is a daily questionnaire representing daily captured enquiry data, but alternatively the time interval may be a weekly questionnaire representing weekly enquiry data, or any other suitable configurable time interval or frequency. The time interval or frequency for presenting the questionnaire may be a function of any time unit such as, but not limited to, days (e.g., once a day or every 3 days etc), weeks (e.g., once a week or every 2 weeks etc), or months (e.g., once per month) etc.

A second time-series chart in the form a time-series plot is provided or rendered in the section indicated in the dotted area designated 544. In this embodiment, the time-series graph plots one or more category deviation parameters representing the overall deviation of the answers in each category in each time-interval.

Further explanation of each time-series chart follows below.

### 3.12 Colour-Coded Time-Series Plot (Colour Heatmap)

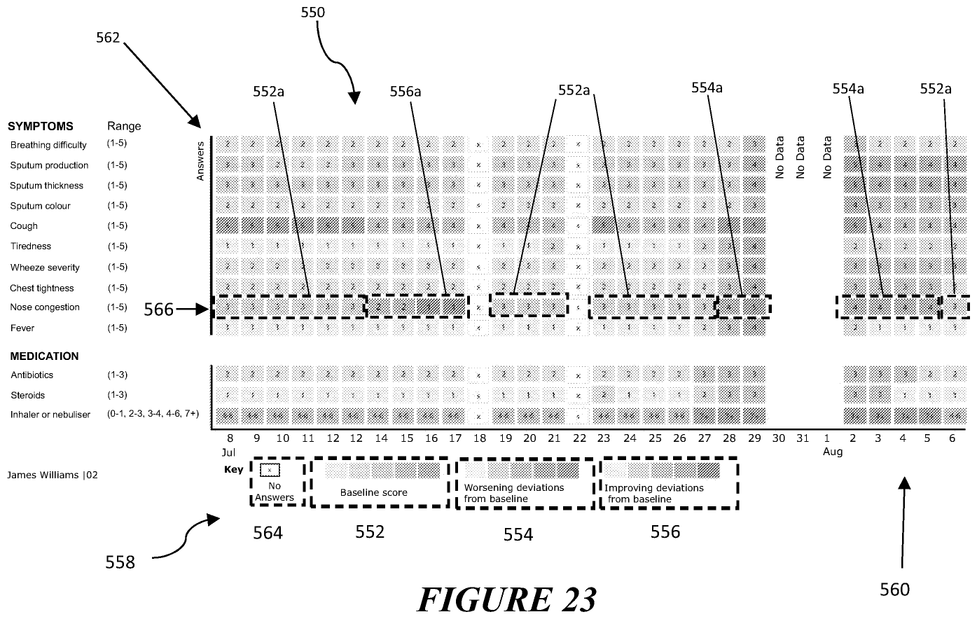

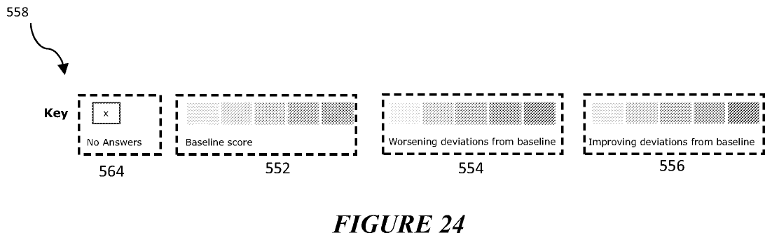

Referring to FIGS. 23 and 24, an example colour-code time-series plot 550 of the type shown in area 546 of the data report of FIG. 22 will be explained in more detail.

### Answer Data

In this example, the colour-coded time-series plot 550 is configured to plot the answer data for a patient for the one or more health parameters over a time period. In this embodiment, the colour-coded time-series plot 550 is in the form of a heatmap, e.g. a colour heatmap. The heatmap may be a cluster heatmap for example.

Each plotted answer of the answer data in the heatmap 550 is selectively colour-coded for display based on a baseline colour spectrum and one or more deviation colour spectrums. The colour spectrum selected for each answer is a function of or based on any deviation of the answer data relative to the baseline data. As explained, the colour-coding data defines the colour spectrum selected for each answer.

In this example, the answer data is selectively colour coded according to one of three colour spectrums: a baseline colour spectrum 552, a worsening deviation colour spectrum 554, and an improvement deviation colour spectrum 556, as indicated in the dotted sections of the key section of heatmap 550. In other examples or configurations, the selective colour coding may be according to a baseline colour spectrum and only one general deviation colour spectrum (which may represent a worsening deviation, an improving deviation, or both).

The baseline colour spectrum 552 represents answer data that corresponds to the baseline data for the patient for the relevant health parameter. The worsening deviation colour spectrum 554 represents a worsening or deterioration of the health parameters represented by the answer data relative to the baseline data. The improvement deviation colour spectrum 556 represents an improving of the health parameters represented by the answer data relative to the baseline data.

The baseline colour spectrum 552 enables a healthcare provider to quickly see how severe a patient's symptom and medication-usage baselines are. The worsening and improvement deviation colour spectrums 554 and 556 enable a healthcare provider to quickly see how severe or improved a patient's super-baseline or sub-baseline symptoms and medication usages are respectively.

In this example, the baseline colour spectrum 552 is visually different or distinctive to the one or more deviation colour spectrums 554,556. The baseline colour spectrum 552 has a high visual contrast to the one or more deviation colour spectrums 554,556. The deviation colour spectrums 554,556 are also visually different or distinctive from each other.

In this example, answer data for a health parameter that corresponds to or matches the baseline data is represented as a plotted answer that is colour-coded according to the baseline colour spectrum 552. Answer data that represents a worsening deviation of a health parameter from the baseline data is represented as a plotted answer that is colour-coded according to the worsening deviation colour spectrum 554. Answer data that represents an improving deviation of a health parameter from the baseline data is represented as a plotted answer that is colour-coded according to the improvement deviation colour spectrum 556.

By way of example, referring to FIG. 23, the colour-coding of the answer data for symptom health parameter ‘nose congestion’ will be explained in further detail. The answer data related to the ‘nose congestion’ health parameter is plotted in the data row indicated generally at 566 along the time-axis 560 of the heatmap 550. The answer data in the data cells indicated within the dotted boxes 552a are those colour-coded into the baseline colour spectrum 552. The answer data in the data cells indicated by the dotted boxes 554a are those colour-coded into the worsening deviation colour spectrum 554. The answer data in the data cells indicated by the dotted box 556a are those colour-coded into the improvement deviation colour spectrum 556.

As discussed, the answer data for each health parameter query comprises or is convertible to a score or rating or level on a scale. As will be appreciated, the answer data for each query (depending on the nature of the query) may be numerical or non-numerical, including Likert scale type answers, and each can directly represent or be mapped or transformed or convertible to a score or rating on a scale.

In one configuration, the scale may be multi-level numerical scale of discrete values. For example, the scale may be a 5-level numerical scale from 0 to 4 (e.g., scores or ratings selected from 0, 1, 2, 3, or 4) or 1 to 5 (e.g., scores or ratings selected from 1, 2, 3, 4 or 5), or any other suitable or desirable multi-level discrete scale, whether numeric or non-numeric.

Each score or rating on the scale corresponds to or is assigned a respective discrete colour value on each of the baseline colour spectrum 552 and the one or more deviation colour spectrums 554,556. For example, if the scale is a 5-level scale from 1 to 5, each respective score 1, 2, 3, 4, and 5 corresponds to or is assigned or mapped to a corresponding discrete colour value in each of the colour spectrums. Queries which comprise non-numerical answer options or Likert scale type answers, are also converted to a score or rating on a scale.

In this embodiment, the size of each of the colour spectrums is at least as large as the largest scale associated with any of the queries. For example, if the largest scale comprises 5-levels, each colour spectrum also have 5-levels of colour value. Queries with answers that are mapped to smaller scales or binary values are still mapped to assigned discrete colour values in the larger colour spectrums. In one configuration, the smaller scales (e.g. a 3-level scale) may be mapped to the middle of a colour spectrum, or may be mapped to the lower or upper end of the scale, or may be equi-spaced mapped across the scale. In another configuration, a query having a binary answer or binary value (e.g. ‘yes’ or ‘no’) may be mapped to the respective extreme end colour values of each colour spectrum.

In this embodiment, each of the colour spectrums 552,554,556 is the same size, i.e. comprises the same number of discrete colour values or levels.

In one configuration, the scale extends from or is defined between a first desirable score or rating or level at one end (e.g. start of scale) to a non-desirable score or rating or level at the other end of the scale (e.g. end of scale). The desirable score or rating or level may represent a good or better health condition for the health parameter associated with a query. The non-desirable score or rating or level may represent a bad or worse health condition for the health parameter associated with a query. Depending on the size of the scale, there are also one or more intermediate scores or ratings or levels that progress between each end score or rating or level.

In this embodiment, each of the baseline colour spectrum 552, and the one or more deviation colour spectrums 554,556 comprises a range of discrete colour values of a base or dominant colour extending from lighter colour values at one end of the spectrum to darker colour values at the other end of the spectrum.

In one configuration, desirable scores or ratings or levels on the scale correspond to or are assigned or mapped to discrete colour values on the baseline colour spectrum 552 and one or more deviation colour spectrums 554,556 that are lighter compared to non-desirable scores or ratings or levels.

In another configuration, desirable scores or ratings or levels on the scale correspond to or are assigned or mapped to discrete colour values on the baseline colour spectrum 552 and one or more deviation colour spectrums 554,556 that are darker compared to the non-desirable scores or ratings or levels.

In another configuration, one or more of the colour spectrum may map or assign desirable scores or ratings or levels to the lighter discrete colour values compared to non-desirable scores or ratings or levels, and one or more of the other colour spectrums may map or assign desirable scores or ratings or levels to the darker discrete colour values compared to the non-desirable scores or ratings or levels. For example, the baseline colour spectrum 552 and the worsening deviation colour spectrum 554 is configured to map or assign desirable scores or ratings or levels to lighter discrete colour values compared to the non-desirable scores or ratings or levels, and the improvement deviation colour spectrum 556 is configured to map or assign desirable scores or ratings or levels to darker discrete colour values compared to non-desirable scores or ratings or levels.

In this embodiment, the base or dominant colour associated with each respective baseline colour spectrum 552 and the one or more deviation colour spectrums 554,556 comprises a configurable or predetermined colour, hue, tint, tone, or shade. By way of example, the base or dominant colour may be selected from any of the following or combination of the following: primary colours; any colour defined or formed from a mixture of primary colours; black; and white. In the accompanying drawings, example colour spectrums are shown (when viewed as colour drawings) that are formed from blue, orange, and green base or dominant colours. If the accompany drawings are viewed (converted or rendered) as black and white drawings or in gray scale, the colour spectrums are less visually distinctive and appear as various shades of black, white, and/or grey, i.e. may be viewed or perceived as ‘shade spectrums’ in this disclosure for example.

In some configurations, the colour spectrums may have associated indicia or text or numerals or markers, representing a position, level, score or rating on an associated scale. In some configurations, the indicia coded to each answer data may be indicative of an improvement or worsening of the answer on the scale. In one example, the indicia or test or numeral or marker may change size dependent on whether it represents an answer that is improving or worsening. In another example, indicia in the form of crosses may be presented to represent worsening, while ticks may represent improving. In some configurations, the number of crosses or ticks may represent the degree of improvement or worsening. In another example, the brightness of the shade may be indicative of improvement or worsening of the answer. For example, a darkening shade may indicate worsening, and a lightening shade may indicate improvement, or vice versa, depending on the colour spectrum.

In one configuration, the base or dominant colour associated with the baseline colour spectrum 552 is a cool colour, hue, tint, tone or shade, and the base or dominant colour of the worsening deviation colour spectrum 554 is a warm colour, hue, tint, tone or shade, and the base or dominant colour associated with the improving deviation colour spectrum 556 is a cool colour, hue, tint, tone or shade.

In one configuration, the colour spectrums 552,554,556 may be defined by a base or dominant colour that is either warm or cool, provided the colour spectrums are all visually distinct or different from each other.

In one configuration, the colour spectrums 552, 554, 556 may be defined by a base or dominant colour that is symbolic or representative of the spectrum itself. For example, the baseline colour spectrum 552 may be defined by a base or dominant colour such as blue or another neutral colour, the worsening colour deviation spectrum 554 may be defined by a base or dominant colour such as red, and the improving deviation colour spectrum 556 may be defined by a base or dominant colour such as green.

As discussed above, each plotted answer in the heatmap 550 is colour coded according to a colour spectrum that is selected dependent on whether the answer data corresponds to or deviates from the baseline data. The answer is colour coded within the selected colour spectrum based on an assigned colour value that corresponds to or is a function of the desirability or position of a score or rating or level associated with the answer data relative to the scale. As discussed, the answer data for each health parameter query comprises or is convertible to a score or rating or level on a scale, and each score or rating or level on the scale corresponds to or is assigned or maps to a respective discrete colour value on each of the baseline colour spectrum 552 and the one or more deviation colour spectrums 554,556. The discrete colour values in each colour spectrum represent a discrete score or rating or level on the scale.

In some embodiments, optional indicia in the form of text or numerals representing each answer, or the score, rating or level of the answer on the scale may be displayed within each plotted colour-coded data cell, as shown in the example heatmap 550 of FIGS. 22 and 23. In other embodiments, the plotted data cells may simply be coloured data cells that are coloured according to the colour coding of the answers they represent.

In one configuration, the scale may be defined by lighter colour values at one end representing desirable scores or ratings or levels to darker colour values representing non-desirable scores or ratings or levels, or vice versa. In some configurations, the colour values within each colour spectrum correspond or are consistent in format, i.e. lighter colour values representing better or more desirable answers and darker values representing worse or non-desirable answers, or vice versa. In other configurations, there may be a mixture of colour spectrum formats, with some colour spectrums progressing from lighter to darker colour values representing a worsening of the answer, and other colour spectrums progressing from lighter to darker values representing an improvement of the answer. In some configurations, the nature of the colour spectrum may determine the nature of the colour spectrum format. For example, the baseline 552 and worsening deviation colour spectrums 552,554 may progress from lighter to darker colour values to represent a worsening of the answer, while the improvement deviation colour spectrum may progress from a lighter to darker colour values to represent an improvement in the answer.

In one configuration, each discrete colour value within each respective colour spectrum is defined by the variance of one or more colour properties of the base or dominant colour. For example, the colour property may be intensity, tint, shade, brightness or the like. In such configurations, each distinct colour value within the colour spectrum is a function of or defined by a respective variance in one or more colour properties of the based or dominant colour. In one example, the colour spectrum may comprise five distinct colour values, each corresponding to a respective one of five distinct ratings, scores or levels on the answer scale. By way of example, if the colour values are defined based on the colour property of brightness, then colour value 1 corresponding to level 1 on the answer scale may correspond to 20% brightness, colour value 2 corresponding to level 2 being 40% brightness, colour value 3 corresponding to level 3 being 60% brightness, colour value 4 corresponding to level 4 being 80% brightness, and colour value 5 corresponding to level 5 being 100% brightness of the base or dominant colour defining the colour spectrum, for example.

As shown in FIG. 23, and a close-up in FIG. 24, in this embodiment each of the baseline colour spectrum 552 and one or more deviation colour spectrums 554,556 are each represented in the data report visually by respective colour keys that depict the discrete colour values of each colour spectrum with respect to their position on the scale associated with the answer data. The keys show the order or progression of each discrete colour value within each respective colour spectrum.

Reverting to FIG. 23, in this embodiment, the colour heatmap 550 comprises answers that are plotted in columns according to a time-interval frequency along the time-axis 560 of the plot and rows along a health-parameter axis 562 according to the one or more health parameters to which the answers relate. The colour heatmap 550 may be considered to be a 2D array or matrix of data cells.

In one configuration, the answer data has associated time-interval data representing the time interval the answer data was input by the user, and wherein the answers are plotted as data cells in an array, stack, or column along the time-axis 560 of the time-series plot according to their respective time interval, and with the answers across time intervals being aligned in rows according to their associated health parameter on the health-parameter axis 562.

In this example, the horizontal time-axis 560 extends over the time period of a month, but it will be appreciated the length or duration of the axis may be configurable by the end user to capture different ranges (specified by start and end dates, or a specific number of days or time window for example) of personal health enquiry data. As shown, the time-axis comprises tick labels representing the time interval in which its respective column of data was captured. In this example, the tick labels represent the date or calendar day on which the data was captured.

In this example, the vertical health-parameter axis 562 defines the respective health parameter query to which the answer data relates. As shown, axis tick labels for each row of data define the health parameter associated with each row of answer data. Optionally, the text or information indicative of the range of the answer scale or response options available to the user for each health parameter query may also be displayed next to the health parameter label.

In this example, the vertical health-parameter axis 562 is configured to group the health parameter labels or rows of answer data by category. As shown, the health parameter queries relating to symptoms are grouped together and labelled with a category heading, and health parameter queries relating to medication are grouped together and labelled with a category heading.

In this example, the heatmap comprises individual plotted data cells that are slightly spaced apart. In alternative configurations, the data cells may be contiguous with no spacing between data cells of the rows and/or columns.

In this example, the answer data is plotted into its corresponding data cell in the heatmap 550 based on its associated time interval and health parameter co-ordinates. The data cell of each answer is colour coded according to its selected colour spectrum based on its deviation, if any, from the baseline data. In this example, an answer is coded to the baseline colour spectrum 552 if it matches the baseline, to the worsening deviation colour spectrum 554 if it is worse than the baseline, and to the improving deviation colour spectrum 556 if it is an improvement over the baseline. The specific colour value assigned to the answer from the selected colour spectrum is then dependent on the colour value assigned to the score, rating or level the answer represents on the answer scale. As previously discussed, the selection of the colour spectrum and assignment of the colour value within the selected colour spectrum is based on the answer data and the baseline data and/or deviation data representing any deviation from the baseline data. As previously discussed, this colour assignment for the data cell may be represented by the colour coding assigned to the processed answer data.

In this example, each data cell is colour coded to display the colour value of its selected colour spectrum, and also optionally displays a representation of the answer (e.g. text, number, score, level or rating) within the data cell. However, it will be appreciated that the data cells may simply be colour coded without any additional representations within the data cell in alternative configurations, as the colour coding itself may represent both the nominal answer relative to the answer scale, and any deviation (e.g. worsening or improvement) relative to the baseline.

The colour-coded data cells representing answers in the heatmap 550 may be any suitable shape or size. In FIG. 23 the individual data cells or data markers are plotted as small boxes, but they may alternatively be any other shape, size, or form.

### Skipped Data and No Data

In this embodiment, the heatmap 550 is also configured to optionally display representations or indications in the data cells indicative of skipped data, i.e. queries or entire enquiries that were skipped by the user. In this example, with reference to FIGS. 23 and 24, the skipped data representation is depicted in the key at 564. As shown in the example heatmap, dates 18 and 22 July comprise skipped enquiries (i.e. all queries skipped), as represented by a cross and no colour coding in each of the data cells for those days. In other scenarios, it may be that only one or a few of the queries for a day are skipped and represented by a cross in the column of data cells representing each day. It will be appreciated that any suitable symbol, text, icon and/or colour coding can be used to depict or represent skipped data.

The skipped data indications in the heatmap may allow a healthcare provider (e.g. doctor) to quickly see if a patient has skipped the questionnaire on a given day but has still used their apparatus for therapy on that day. If a patient who normally completes the questionnaire choses to skip it for a series of consecutive days, this could indicate that their condition has rapidly deteriorated, which may be a trigger for their healthcare provider to intervene.

In some configurations, an increase in questionnaire or patient health enquiry compliance by a patient may be detected or identified based on the enquiry data comprising the answer data and any skipped data (i.e., skipped enquiries and/or skipped queries within enquiries). For example, a patient may change from a lower compliance or interaction with completing the questionnaire to a higher compliance or interaction with completing the questionnaire, and this change may be detected or identified. In one configuration, lower compliance with completing the questionnaire may be represented by a high level or frequency of skipped enquiries and/or a consecutive series of skipped enquiries over a specific time period (e.g., specific number of consecutive days or weeks or other relevant time interval) for example. A higher compliance with completing the questionnaire may be represented by a low rate or frequency of skipped enquiries and/or a consecutive series of completed or at least partially completed enquiries over a specific time period (e.g., specific number of consecutive days or weeks or other relevant time interval) for example.

In some embodiments, the data server 506 may process the enquiry data (e.g., answer data and any skipped data) to identify or detect any significant changes in enquiry completion compliance. For example, the data server 506 may be configured to identify a significant increase in enquiry completion compliance based on one or more thresholds, and/or if the patient moves from a low-compliance status or category to a high-compliance status or category based on either objective criteria or relative to their own baseline or historical data for enquiry completion compliance. These thresholds and/or categories and/or criteria for identifying significant changes in enquiry completion compliance may be pre-programmed or configurable by a healthcare provider for example. The date and/or time data associated with any such detected change in enquiry completion behaviour may be recorded or marked for subsequent reporting, alerts and/or notifications to a healthcare provider. For example, an increase in enquiry compliance could indicate a worsening in the patient's condition. Patients may be more likely to engage with the patient health enquiry when they are feeling worse than usual.

In one example configuration, the data server 506 may generate data indicating the detection of a change (e.g., increase) in enquiry completion compliance for the patient, and may flag, mark, and/or notify this change in patient compliance or engagement behaviour visually in any of the generated data reports, time-series plots (e.g., heatmap), and/or time-series graph, or may send a message, alert or notification to the healthcare provider indicating the change. In one example, the column of answer data or data cells in the heatmap 550 for the day or time interval associated with the detected increase in enquiry completion behaviour may be highlighted, shaded, or otherwise visually distinguished or marked with a symbol, overlay element, background element, or graphical indications to alert a healthcare provider to the change. In another example, a message or notification of the change in behaviour (and therefore the potential worsening of the patient's condition) may be displayed on the healthcare providers dashboard or homepage when they log-in or otherwise access a healthcare provider portal of the data server 506 (e.g., patient and device management platform).

In this embodiment, the heatmap 550 is also configured to optionally display representations or indications in the data cells to indicate ‘no data’ or ‘no device interaction’. Referring to FIG. 23, dates 30 July to 1 August depict textual representations that no data is associated with those days. This ‘no data’ indication is distinct from the skipped data. Skipped data represents that the user or patient has interacted with their breathing assistance apparatus that day, but has elected to skip the enquiry or specific queries within the enquiry. In contrast, a ‘no data’ indicates that the user or patient did not interact (e.g. start or use) the breathing assistance apparatus at all during that day. The heatmap may represent this non-interaction with any form of suitable no-data graphical indicator, whether textual, symbols, icons, markers, colour coding or the like. In the example shown in FIG. 23, the no-data graphical indicators are depicted by blank columns of data cells for the relevant days or time intervals and a textual indicator ‘No Data’.

The no-data indicators enable a healthcare provider to quickly see if a patient has not interacted with their breathing assistance apparatus at all on a given day. This suggests that the patient may have been hospitalised, which could indicate to the healthcare provider that the apparatus settings need to be adjusted when the patient returns home and starts using their apparatus again. The no-data indicators enable a healthcare provider to assess the patient's engagement with the breathing assistance apparatus. The healthcare provider may be able to quicky determine that there has been no use of the apparatus over one or multiple successive days based on the displayed no-data indicators in the chart, and may take appropriate actions based on this acquired knowledge.

In some configurations, the data server 506 is configured to receive data from the breathing assistance apparatus indicating which time intervals had no-user interaction, i.e. no data. This may be a separate data set sent when the breathing assistance apparatus is next ‘online’ and in data communication with the data server. Alternatively, the no data information may be deduced or determined by the data server if no enquiry data is received for a specific time interval or intervals. In one configuration, each time interval (e.g. day) may have a default ‘no data’ status, until this is subsequently updated with a set of enquiry data received at the data server.

In some embodiments, the colour coding of the answer data based on the baseline data and/or deviation data represents a compressed data set of the answer data and baseline data. The colour coding data associated with each answer encodes both the answer and any deviation relative to the baseline. For example, the colour value coded to the answer can represent both the selected colour spectrum (indicating any deviation from baseline) and the actual answer on the answer scale (as each colour value is mapped to a rating, score or level on the answer scale).

Patient Physiological Parameter Data, Therapy Setting Data, and/or Gases Flow Data

In some embodiments, any of the time-series charts disclosed herein (e.g., colour-coded time-series plot or colour heatmaps such as 550, and/or time-series plots or graphs such as 570) may be configured to additionally plot data or data types relating to or indicative of one or more measured, sensed, or determined patient parameters, and/or therapy settings, and/or gases flow characteristics.

By way of example, as previously explained, the controller of the apparatus may be configured to sense, receive or determine data over time indicative of one or more physiological parameters of the patient such as respiratory rate, oxygen saturation, tidal volume, spirometry data and/or other related parameters or indexes (e.g., ROX index), and/or therapy setting data and/or gases flow data (e.g. pressure, flow rate, fraction of inspired oxygen FiO₂ data, and/or fraction of delivered oxygen FdO₂ data).

In one configuration, the colour heatmaps such as 550 may additionally plot any one or more of the patient parameter data and/or apparatus therapy setting data and/or gases flow data alongside the answer data from the patient health enquiry in the colour heatmap.

In one configuration, the one or more additional data rows in the heatmap for the patient parameter data, therapy setting data, and/or gases flow data may be grouped and plotted under their own category heading in the colour heatmap such as ‘patient parameters’, ‘therapy settings’, ‘gases flow parameters’ or the like, similar to the format used for the symptom and medication data rows and headings of the heatmap. In other configurations, the patient parameter data, therapy setting data, and/or gases flow data may be plotted against the time axis 560 at any other suitable or desired position or data row along the vertical axis 562 of the colour heatmap 550.

In one configuration, the time-series graphs or plots such as 570 (e.g., line and/or bar plots or graphs) may additionally plot any one or more of the patient parameter data and/or apparatus therapy setting data or gases flow data as extra lines and/or bar plots alongside the answer data from the patient health enquiry.

Some specific examples of the plotting or rendering of this additional data in their own respective rows in the colour heatmap 550 alongside the answer data to the patient health enquiry are described below, and it will be appreciated that the examples could be applied or used for any of the additional data types described. It will also be appreciated that any of the additional data types mentioned above or in the examples below (e.g., patient parameters, therapy settings, and/or gas flow parameters) may also be plotted in any of the other time-series graphs (e.g., the bar and/or line plots such as 570).

In one embodiment, the system (e.g., patient and device management platform or dashboard) is provided with a user interface that enables a user, such as a healthcare provider, to select or configure which parameters to display or plot in the time-series charts (e.g., colour heatmaps such as 550 and/or time-series graphs such as 570). For example, the healthcare provider may configure which health parameters from the symptoms and medication categories to display. The healthcare provider may also configure which one or more of the additional data types or parameters to plot or display in the time-series charts from the measured or determined patient parameter data and/or apparatus therapy setting data and/or gases flow data, examples of which are described above and further below.

### Respiratory Rate Data

In one example configuration, the colour heatmap 550 may be configured to additionally plot data representing the patient's respiratory rate in a data row of the heatmap. In one example, the raw respiratory rate data may be processed, transformed, mapped or converted into a format suitable for plotting into the data cells of a row of the heatmap. In one configuration, respiratory rate data of a patient for a therapy session or discrete time interval corresponding to the heatmap time-axis may be converted into a discrete respiratory rate value representative of that therapy session or time interval. In one example, the discrete respiratory rate value may represent the average respiratory rate of the patient over the therapy session or other discrete time interval corresponding to the data cells of the colour heatmap.

The discrete respiratory rate value may be plotted into is associated data cell in the heatmap in raw format (e.g., a numerical average respiratory rate value), or it may be converted or transformed into a score or rating on a scale, similar to the answer data scale previously described. By way of example, the average respiratory rate value may be categorised or mapped into a discrete respiratory rate score or rating on a multi-level rating scale of the type previously described. In one configuration, respiratory ratings at one end of the scale may represent a good or healthy respiratory rate and ratings at the other end of the scale may represent an unhealthy respiratory rate. For example, the average respiratory rate value for a therapy session or time interval may be mapped or transformed to a 5-level scale (e.g., 0, 1, 2, 3, 4) or any other suitable rating scale of the type previously described in relation to the answer data of the patient health enquiry.

In some configurations, the respiratory rate score or rating data may be plotted into its respective data cell of the respiratory rate data row in the colour heatmap 550. In some configurations, each respiratory rate score or rating may also be colour-coded for display based on the baseline colour spectrum and one or more deviation colour spectrums, in a similar manner to the answer data of the patient health enquiry. For example, the stored baseline data for the patient may include baseline respiratory rate data (e.g., the patient's average respiratory rate for a therapy session or relevant time interval). Each respiratory rate score may be colour-coded for display in the colour heatmap 550 as a function of or based on any deviation of the respiratory rate score relative to the patient's baseline respiratory rate score data.

Additionally or alternatively, the respiratory rate data may be plotted or displayed in any of the time-series graphs (e.g., bar and/or line plots such as 570) with its own respective bar or line or other plotted indicator or marker.

Oxygen Saturation Data (e.g., SpO₂ Data), FiO₂ Data, ROX Data

In one example configuration, the colour heatmap 550 may be configured to additionally plot data representing the patient's oxygen saturation (e.g., SpO₂ data) in a data row of the heatmap. As previously described above, the SpO₂ data may be provided to the controller by an external sensor such as, but not limited to, a pulse oximeter or a wearable sensor. Like the respiratory rate data example, the raw SpO₂ data may be processed, transformed, mapped or converted into a format suitable for plotting into the data cells of a row of the heatmap. In one configuration, SpO₂ data of a patient for a therapy session or discrete time interval corresponding to the heatmap time-axis may be converted into a discrete SpO2 value representative of that therapy session or time interval. In one example, the discrete SpO2 value may represent the average SpO₂ of the patient over the therapy session or other discrete time interval corresponding to the data cells of the colour heatmap.

Like the respiratory rate data example, the discrete SpO₂ value may be plotted into its associated data cell in the heatmap in raw format (e.g., a numerical average SpO₂ value), or it may be converted, transformed, or mapped into a score or rating on a scale. The scale may be a multi-level rating scale (e.g., 5-level scale of 0, 1, 2, 3, 4) with one end of the scale representing a good or healthy SpO₂ and the other end of the scale representing an unhealthy SpO₂.

In some configurations, the SpO₂ score or rating data may be plotted into its respective data cell of the SpO₂ data row in the colour heatmap 550. In other configurations, each SpO₂ score or rating may also be colour-coded for display based on the baseline colour spectrum and one or more deviation colour spectrums, in a similar manner to the answer data of the patient health enquiry. For example, the baseline data for the patient may include baseline SpO₂ data (e.g., the patient's average SpO₂ for a therapy session or relevant time interval). Each SpO₂ score may be colour-coded for display in the colour heatmap 550 as a function of or based on any deviation of the SpO₂ score relative to the patient's baseline SpO₂ score data.

In another example configuration, the colour heatmap 550 may be configured to additionally plot data representing the fraction of inspired oxygen FiO₂ and/or ROX index in a respective data row or rows of the heatmap. In such configurations, the plotted FiO₂ and/or ROX index data in the heatmap may be more informative in scenarios where the controller of the apparatus is configured to maintain or keep the SpO₂ constant or is targeting an SpO₂ set point. By way of example, displaying FiO₂ data in the heatmap may illustrate how much oxygen the apparatus is having to provide during closed loop flow therapy in order to maintain a target SpO₂ set point. Likewise for plotting the ROX index data, as the ROX index value as a function of FiO₂. In particular, the ROX index data is calculated with the following equation:

$$ROX = \frac{{SpO}_{2} / {FiO}_{2}}{RR}$$

Where RR is respiratory rate.

The FiO₂ data or ROX index data may be plotted in respective data rows of the heatmap in the same manner or formats described with respect to the respiratory rate data and SpO₂ data described above. For example, the FiO₂ data or ROX index data may be averaged for a therapy session or relevant time interval to generate a discrete FiO₂ data value and/or discrete ROX index data value. The discrete FiO₂ values or ROX index values may then be plotted in raw format into the heatmap, or may be categorised, mapped or transformed into a respective FiO₂ score or rating or ROX index score or rating according to a relevant multi-level rating scale of the type previously described. The FiO₂ score and/or ROX index score may then be plotted into respective data cells of a respective FiO₂ data row and/or ROX index data row of the heatmap. In some configurations, the FiO₂ score and/or ROX index score may be colour-coded for display based on the baseline colour spectrum and one or more deviation colour spectrums, based on any deviation of the scores relative to stored respective FiO₂ and/or ROX index baseline data for the patient.

Additionally or alternatively, the SpO₂ data, FiO₂ data, and/or ROX index data may be plotted or displayed in any of the time-series graphs (e.g., bar and/or line plots such as 570) with its own respective bar or line or other plotted indicator or marker.

### Tidal Volume Data

In some configurations, the breathing assistance apparatus may also be selectively operable in an NIV mode to provide a flow of gases for NIV therapy. In such configurations, the controller of the apparatus may be configured to sense, measure or determine tidal volume data representing or indicative of the tidal volume of the patient during a therapy session.

In another example configuration, the colour heatmap 550 may be configured to additionally plot data representing the patient's tidal volume in a data row of the heatmap. Similar to any of the examples above, the tidal volume data may be converted into a discrete tidal volume value for each therapy session or relevant time interval (e.g. an average tidal volume per therapy session or relevant time interval may be determined). In one configuration, the discrete tidal volume values may then be plotted as raw numerical values in their respective data cells of the tidal volume data row in the heatmap. In another configuration, the discrete tidal volume values may be categorised, transformed or mapped to a corresponding tidal volume score or rating according to a relevant multi-level rating scale of the type previously described. The tidal volume score or rating may then be plotted into respective data cells in the tidal volume data row of the heatmap. In some configurations, the tidal volume scores may be colour-coded for display based on the baseline colour spectrum and one or more deviation colour spectrums, based on any deviation of the scores relative to stored tidal volume baseline data for the patient, in a manner similar to the other displayed data.

In some embodiments, if the breathing assistance apparatus is operating in NIV mode to deliver NIV therapy, the controller of the apparatus may be configured to determine respiratory rate data for the patient based on the detected inspiratory and expiratory phases of the patient's respiratory cycle, and this controller-derived respiratory rate data may be plotted in a respiratory rate data row of the colour heatmap or as a bar or line in the time-series graph in a manner similar to that described in the previous respiratory rate data example above.

Additionally or alternatively, the patient's tidal volume data may be plotted or displayed in any of the time-series graphs (e.g., bar and/or line plots such as 570) with its own respective bar or line or other plotted indicator or marker.

Spirometry and/or Lung Performance Data

In one example configuration, the colour heatmap 550 may be configured to additionally plot spirometry or spirometry-type data representing lung performance measurements or lung function diagnostics for a patient.

In one embodiment, the breathing assistance apparatus and/or data server may be operatively connected to an off-the-shelf or external spirometer device or system, and configured to receive spirometer data or other lung function measurement data determined or sensed by the spirometer device or system. The breathing assistance apparatus and/or data server may be operatively connected to the spirometer device or system via a wired or wireless data connection for example.

In another embodiment, the breathing assistance apparatus may be operable in a diagnostic or spirometry mode or as a spirometry device. In one example, the breathing assistance apparatus may be configured to operate in a spirometry mode with the use of a removable measurement device or accessory component that is attachable to the end of the breathing conduit. The patient may perform expiratory manoeuvres into the measurement device against a flow of gases. Spirometry data and/or lung performance measurement data may be extracted from sensor data generated by apparatus sensors measuring one or more characteristics of the flow of gases during the expiratory manoeuvres.

The lung performance measurement or spirometry data from either of the above configurations may be received, stored and processed for display within the heatmap 550 alongside the other heatmap data. Any one or more useful lung performance measurement or spirometry data, values or features may be processed and displayed. Such features or data may include, but are not limited to, peak flow and tidal volume, for example.

In some embodiments, the lung performance measurements or spirometry measurements may be performed prior, during or after the patient health enquiry or questionnaire, or as part of the questionnaire process. In some embodiments, each set of enquiry data for the questionnaire may comprise an associated set of lung performance measurement or spirometry data, i.e. the lung performance measurements or spirometry data may be captured at the same frequency as the questionnaire data. In other embodiments, the lung performance measurements or spirometry data may be captured with a lower frequency than questionnaire data, such that only some questionnaires have associated lung performance or spirometry data, such that the data is updated less frequency that the enquiry data.

In an embodiment, the one or more lung performance measurement or spirometry data features may be plotted and/or represented in a respective lung performance or spirometer data row of the heatmap 550. Similar to any of the examples above, the lung performance measurement or spirometry data may be converted into a discrete lung performance measurement or spirometry value for each relevant time interval (e.g., a discrete peak flow value or tidal volume value for or representing a particular time interval, e.g., a specific day or week represented in the heatmap).

In one configuration, the discrete lung performance measurement or spirometry data values may then be plotted as raw numerical values in their respective data cells of the lung performance measurement or spirometry data row(s) in the heatmap. In another configuration, the discrete lung performance or spirometry data values may be categorised, transformed or mapped to a corresponding lung performance measurement or spirometry score or rating (e.g., peak flow score and/or tidal volume score) according to a relevant multi-level rating scale of the type previously described. The one or more lung performance measurement or spirometry scores or ratings may then be plotted into respective data cells (according to the time period or interval they were captured) in their respective lung performance measurement or spirometry data rows of the heatmap. In some configurations, the one or more lung performance measurement or spirometry scores may be colour-coded for display based on the baseline colour spectrum and one or more deviation colour spectrums, based on any deviation of the scores relative to stored lung performance measurement or spirometry baseline data for the patient, in a manner similar to the other displayed data.

Additionally or alternatively, one or more of the lung performance measures or spirometry data may be plotted or displayed in any of the time-series graphs (e.g., bar and/or line plots such as 570) with its own respective bar or line or other plotted indicator or marker.

### Medication Prompts

Patients are often given or prescribed antibiotics or other medications when they are discharged or sent home from hospital or after visiting healthcare facilities. However, they may choose to only use them or may be instructed to only use them if they start to feel worse.

In some configurations, the enquiry data may be processed by the breathing assistance apparatus and/or data server to determine if the patient's health condition is worsening (based on one or more thresholds, criteria, and/or trend analysis). If a worsening condition is detected or determined based on the enquiry data, then the controller of the breathing apparatus may be triggered to prompt the patient to start using their prescribed medication via a notification or message on the display of the apparatus or a message or notification sent or pushed to the patient's smartphone, tablet, wearable, computer or other electronic device.

Additionally or alternatively, the detection of a worsening condition may trigger a message, notification or alert to the patient's healthcare provider to consider contacting the patient to recommend they commence taking the medication or to prescribe the required or suggested medication to the patient. The healthcare provider alerts or notifications may be displayed on a dashboard or notification panel of their interface to the patient and device management platform, or otherwise pushed or sent directly to an electronic device of the healthcare provider (e.g., computer, smartphone, tablet, wearable or the like).

The time stamp (e.g. time interval, time, and/or day) associated with the triggered patient and/or healthcare provider medication prompt may also be marked or displayed or highlighted as a feature or graphical element in a data cell of the heatmap such as 550 or as an indicator or marker on the time-series graph such as 570, or otherwise plotted as a data point or marker or indication relative to the time-line axis of the heatmap and/or time-series graph.

### Prompt to Contact Healthcare Provider

In some configurations, the breathing assistance apparatus may prompt the patient to contact their healthcare provider in response to certain changes in their enquiry data and/or time-series charts (e.g., heatmap such as 550 and/or time-series graphs such as 570). For example, the prompt may visual and/or audible. The prompt may be displayed on the breathing assistance apparatus and/or pushed to a patient's personal electronic device (e.g., smartphone, tablet, wearable, computer) in the form of a notification, alert, or message, and/or an audible notification, alert or message.

In one example embodiment, the enquiry data and/or time-series chart (e.g. heatmap and/or time-series graph) is processed by the breathing assistance apparatus and/or data server (e.g., patent and device management platform), and the prompt to contact their healthcare provider may be triggered if the data meets one or more predetermined or configurable triggering criteria or thresholds. In some scenarios, the triggering criteria may specify a general worsening of the patient's condition (e.g., 3 or more worsening health parameters) or the triggering criteria may relate to the worsening of a selected one or more health parameters by a specified degree. The triggering criteria may be general or patient-specific. The triggering criteria may be customised or configured for each patient or a category, group, or class of similar patients.

### Hospital Stay Data

As described earlier, in some configurations, the personal health enquiry may present a query or queries to the patient about hospitalisation or hospital visits. The response or answer data entered by the patient may comprise hospitalisation date data indicative or representative of the dates and/or time periods the patient was in hospital receiving respiratory-related treatment.

In some configurations, this hospitalisation date data may be presented, displayed and/or represented in the colour heatmap 550. In one example, the column or columns of data or data cells in the heatmap relating to the days, weeks or other relevant time interval in which the patient was in the hospital according to the hospitalisation date data may be highlighted, shaded, or otherwise visually distinguished or marked with a symbol, overlay element, background element, or other graphical indications or markers. This may provide a healthcare provider with additional context to the heatmap enquiry data, as they can visually distinguish the patient's colour-coded answer data in the heatmap from between when they are at home (or any other non-hospital location) and in hospital.

Additionally or alternatively, the hospitalisation date data may be plotted or displayed in any of the time-series graphs (e.g., bar and/or line plots such as 570) with its own respective bar or line or other plotted indicator or marker.

### Queries Presented in Enquiry Based on Patient Parameter Data

In some configurations, the queries selected for presentation in the personal health enquiry may be at least partly based on or a function of any one or more aspects of sensed, measured or determined patient physiological parameter data such as, but not limited to, those discussed above. For example, some of the queries presented in the personal health enquiry may be at least partly based on or a function of any one or more of the following: respiratory rate data, SpO₂ data, FiO₂ data, ROX data, tidal volume data, spirometry or lung performance data, or any other measured or sensed patient parameter data. In one example, some of the queries may be triggered for presentation or inclusion in the personal health enquiry based on or as a function of one or more of these example patient parameters satisfying associated trigger thresholds, criteria or conditions. For example, some of the queries may be presented in the personal health enquiry conditional upon one or more aspects of the patient parameter data satisfying pre-programmed or configurable trigger criteria or conditions. In such configurations, the content or set of queries in the personal health enquiry may be at least partly dependent or a function of one or more aspects of the measured or determined patient parameter data examples.

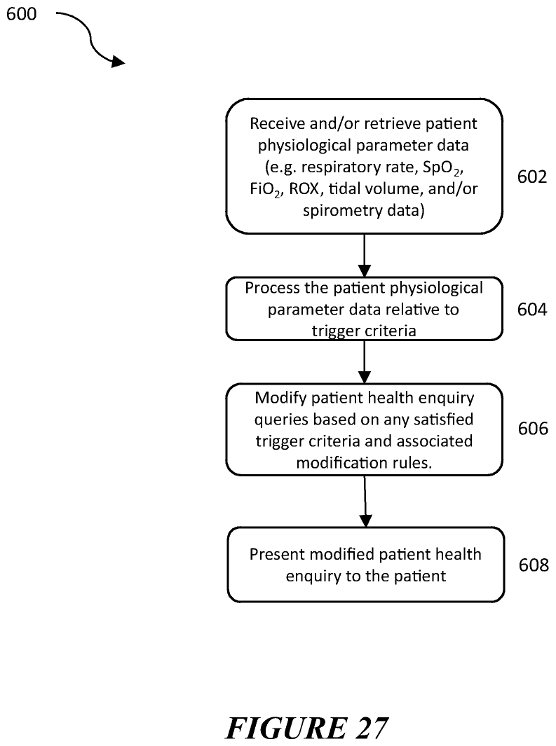

One example configuration of the above process will be explained with reference to FIG. 27. The example process 600 in FIG. 27 initiates with the processor (e.g. a processor the data server and/or breathing assistance apparatus) receiving or retrieving one or more aspects of patient physiological parameter data such as, but not limited to, any one or more of: respiratory rate data, SpO₂ data, FiO₂ data, ROX data, tidal volume data, spirometry or lung performance data, or any other measured or sensed patient physiological parameter data, as shown at step 602. The received or retrieved patient physiological parameter data is then processed relative to one or more respective trigger criteria as shown at step 604. If any of the trigger criteria are satisfied by the patient physiological parameter data, then the controller is configured to modify the patient health enquiry that is to be presented to the patient in accordance with stored respective modification rules associated with the trigger criteria, as shown at step 606. For example, the trigger criteria associated with a certain parameter may comprise a modification rule that dictates one or more additional queries to be inserted into the enquiry, and/or deletion of certain queries, if the trigger criteria are satisfied. Once any necessary modifications to the patient health enquiry are made, the modified health enquiry may then be presented to the patient either immediately, at the next scheduled questionnaire time, or at some other configurable time, as shown at step 608.

### Data Processing

The processing of the patient parameter data, therapy setting data, gases flow data, and/or any other data for display or rendering in the time-series charts (e.g., heatmaps such as 550 and time-series graphs such as 570) and/or triggering of alerts or notifications or prompts, as described in the above examples, may be carried out by the controller of the breathing assistance apparatus and/or the data server (e.g., the patient and device management platform).

### 3.13 Time-Series Graph

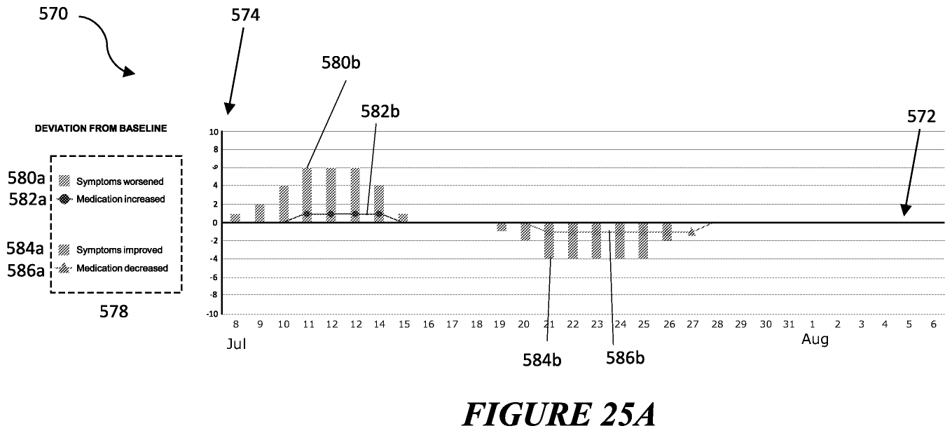

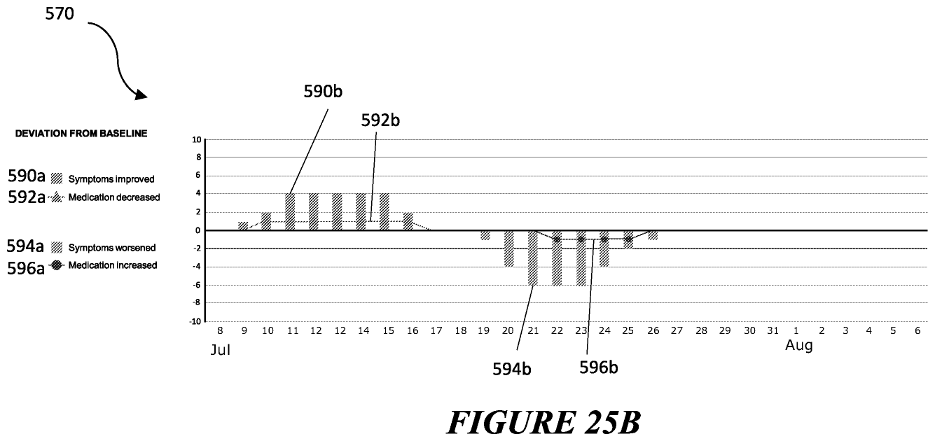

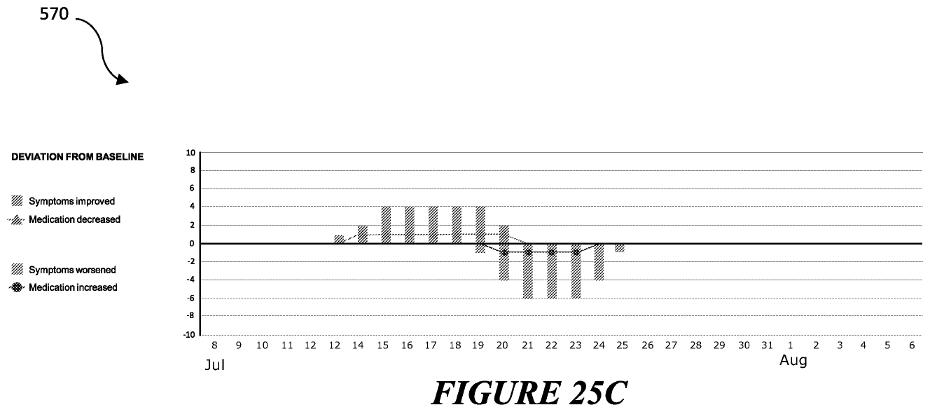

Referring to FIGS. 25A-25C, examples of a time-series graph 570 of the type shown in area 544 of the data report of FIG. 22 will be explained in more detail.

In this example, the time-series graphs 570 are configured to plot one or more category deviation parameters or values calculated based on or represented by the deviation data. As discussed, the deviation data represents any deviation in the answer data from the baseline for each health parameter query.

In this example, the horizontal-axis is a time-axis 572 of a similar nature and configuration to that described in respect of the heatmap 550. In particular, the time-axis represents a time-series over a time period (e.g. month) and with tick labels representing individual time intervals (e.g. days or dates) within the time period associated with the captured enquiry data. The vertical-axis is a deviation value axis 570 representing a category deviation parameter.

In this example, the deviation data represents symptom data deviation and/or medication data deviation in the form of one or more category deviation parameters for plotting that represent health parameters in each respective category that have deviated from their respective baseline data for each time-interval. The time-series graph 570 plots the one or more category deviation parameters for the time intervals over the time period.

In this example, the data server 506 is configured to generate a symptom category deviation parameter or parameters representing or being a function of the health parameters that deviate from their baseline for each time-interval, and a medication category deviation parameter or parameters representing or being a function of the health parameters that deviate from their baseline for each time-interval.

In one configuration, each category has a category improvement deviation parameter and a category worsening deviation parameter. The category improvement deviation parameter represents or is a function of the number of health parameters with an improved deviation relative to the baseline. The category worsening deviation parameter represents the number of health parameters with a worsening deviation relative to the baseline. For example, in one configuration, if the symptoms category had two answers with improved deviations relative to the baseline, the symptom category improvement deviation parameter will be two. Likewise, if the symptoms category had three answers with worsening deviations relative to the baseline, the symptom category worsening deviation parameter will be three.

In one configuration, the category improvement deviation parameters and category worsening deviation parameters may have opposite polarities assigned, to differentiate them when plotting the parameters in the graph 570. For example, in one configuration, the category improvement deviation parameters may be assigned a positive polarity, and the category worsening deviation parameters may be assigned a negative polarity. In another configuration, the category improvement deviation parameters may be assigned a negative polarity, and the category worsening deviation parameters may be assigned a positive polarity.

In one configuration, the data server 506 is configured to calculate or determine the category deviation parameters for each time-interval as a function of or based on a colour-coding of the answer data related to the heatmap 550. For example, the category deviation parameters for each time-interval are based on or determined as a function of the number of answers in each respective category that are colour coded into the worsening deviation colour spectrum and/or improving deviation colour spectrum. For example, with reference to the heatmap in FIG. 23, the symptom category worsening deviation parameter for 28 July is 6, represented by the 6 data cells coded into the worsening deviation colour spectrum 554 for 28 July, and the symptom category improvement deviation parameter for days 14-17 July is 1, represented by the 1 data cell coded into the improving deviation colour spectrum 556 for those days.

In another configuration, the category deviation parameters may be independently calculated or determined as an independent data set for each time interval from the answer data, baseline data and/or deviation data, without reference to the colour coding data.

As shown in FIGS. 25A-25C, the data server is configured to generate or render a time-series graph 570 that plots both symptom category deviation parameter(s) (improving and worsening) and medication category deviation parameter(s) (improving and worsening). The plots of the symptom category deviation parameter(s) and medication category deviation parameter(s) are visually distinguishable from each other or visually represented as separate data sets within the same time-series graph (i.e. plotted with respect to the same vertical and horizontal axes 572,574).

In one configuration, the plots of the symptom category deviation parameter(s) and medication category deviation parameter(s) are represented by the same or different types of plots.

In one configuration, the symptom category deviation parameter(s) and medication category deviation parameter(s) are represented by independent plots within the same time-series graph or as combined plots within the same time-series graph.

The plots of the symptom category deviation parameter(s) and medication category deviation parameter(s) are selected from any one or more of the following types of plots: dot plot, line chart or line graph, bar chart, lollipop chart, stacked bar chart, and grouped bar chart.

In one configuration, the time-series graph comprises the symptom category deviation parameter(s) being plotted as bar charts and the medication category deviation parameter(s) are plotted as line charts (as shown in FIGS. 25A-25C), or vice versa.

As shown in the sample data of FIGS. 25A-25C, the bar charts and line charts representing the symptom category deviation parameter(s) and medication category deviation parameter(s) are plotted relative to the same deviation axis 574 such that they may overlay each other visually.

In one configuration, the bar charts and line charts representing the symptom category deviation parameter(s) and medication category deviation parameter(s) are further visually distinguished from each other by colour, pattern, or shade.

A key indicated at 578 in FIG. 25A may define the type of chart and any other distinguishing features or characteristics (e.g. colour, pattern, or shade) for the plots of each of the respective category deviation parameters. By way of example, in FIG. 25A, key item 580a for ‘symptoms worsened’ in the key is linked or corresponds to the bar charts coloured like bar 580b. Key item 582a for ‘medication increased’ in the key is linked or corresponds to the line charts coloured like line 582b. Key item 584a for ‘symptoms improved’ in the key is linked or corresponds to the bar charts coloured like bar 584b. Key item 586a for ‘medication decreased’ in the key is linked or corresponds to the line charts coloured like line 586b.

In the example of FIG. 25A, the category worsening deviation parameters for each of the symptoms and medication categories are assigned a positive polarity with respect to the deviation axis 574 such that they are plotted above the horizontal axis 572. The category improving deviation parameters for each of the symptoms and medication categories are assigned a negative polarity such that they are plotted below the horizontal axis 572. FIGS. 25B and 25C shows an example of an opposite polarity assignment to FIG. 25A, such that improvement parameters are plotted above the horizontal axis, and worsening parameters are plotted below the horizontal axis 572. By way of example, in FIGS. 25B and 25C, key item 590a for ‘symptoms improved’ in the key is linked or corresponds to the bar charts coloured like bar 590b. Key item 592a for ‘medication decreased’ in the key is linked or corresponds to the line charts coloured like line 592b. Key item 594a for ‘symptoms worsened’ in the key is linked or corresponds to the bar charts coloured like bar 594b. Key item 596a for ‘medication increased’ in the key is linked or corresponds to the line charts coloured like line 596b.

FIG. 25C shows an example of sample data in which there are time intervals (days 19 and 20 July) in which there is a mixture of improvements and worsening of one or more health parameters in each category in the same day.

The time-series graphs 570 may also optionally be configured to display representations and/or indicators of time intervals containing skipped data, and/or no data, in a similar manner to that described with respect to the heatmap. By way of example, FIG. 22 shows in area 544 a version of a time-series graph of the category deviation parameter(s) which depicts time intervals (e.g. days) with skipped data with graphical indicators in the form of dotted bars (see days 19 and 22 July), and days with no data (i.e. the user or patient did not interact with the breathing assistance apparatus) with graphical indications in the form of striped bars (see days 30 July-1 August). It will be appreciated that any suitable form of graphical indication or representation may be used in the plot, and this need not be bars.

In one configuration, the time-series graphs 570 may further be configured to depict or plot graphical indicators or representations indicating time intervals or days upon which an updated patient treatment prescription occurred or when the prescription changed. For example, the data server may be configured to receive or retrieve prescription data or may otherwise determine when the prescription has changed, and may plot or mark this against the relevant time interval (e.g. day) on the time-axis of the graph.

### 3.14 Graphical Dashboard of Data for Multiple Patients

As described with reference to FIG. 20, the patient and device management platform hosted by the data server 506 is configured to receive and process data for multiple patients. As previously discussed, a healthcare provider may access the patient and device management platform to access personal health enquiry data associated with their patients or a sub-set of their patients. The data server 506 is configured with access rights enabling or permitting the healthcare provider to only access data associated with their patients, for example based on authorization data.

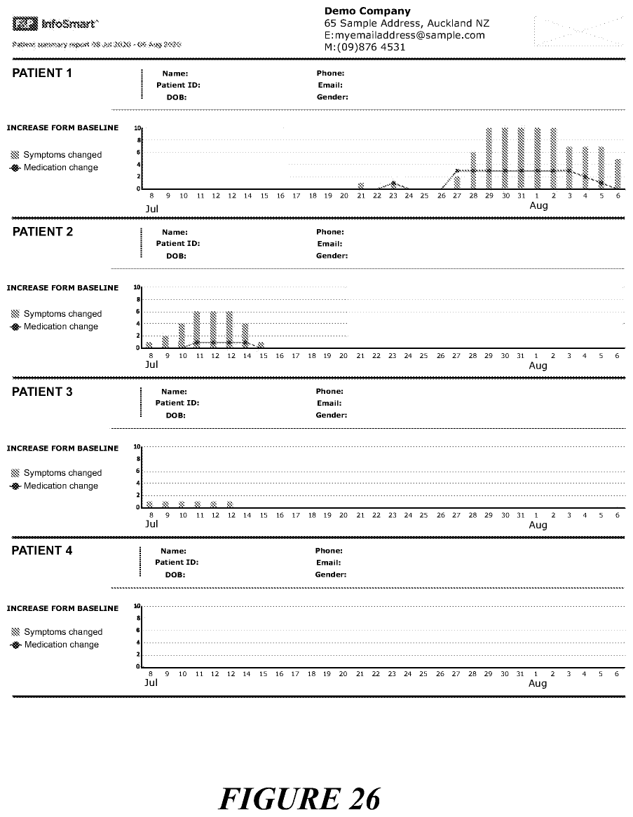

Referring to FIG. 26, in one configuration, the healthcare provider may be configured to access a graphical dashboard that depicts a summary time-series chart for each of their patients. By way of example, FIG. 26 shows a graphical dashboard showing the time-series graph of the type described with respect to FIGS. 25A-25C for each patient in a group of four patients being monitored by the healthcare provider. In alternative configurations, the summary time-series chart may be the colour heatmap for each patient described in relation to FIGS. 23 and 24. In a further alternative configuration, the summary for each patient may display both their times-series graph and colour heatmap.

In one configuration, the graphical dashboard may comprise a configurable sorting or ordering setting or settings. For example, the healthcare provider may configure the graphical dashboard to layout or order the presentation of the patient summary graphs at least partly based on a determined priority status or other sorting criteria.

In some embodiments, the patient data may be sorted or ordered according to a single sorting criteria. In some embodiments, there may be a plurality or multiple cascading sorting criteria. For example, the patients may be ordered first in regard to a first sorting parameter or setting, and ordered secondly with regard to a second sorting parameter, and so on.

In one example, the data server is configured to process the enquiry data (including answer data, skipped data, and no data) and baseline data to determine a priority ranking amongst a group of patients to determine a ranking or priority order in regard health condition or potential for exacerbation or some other priority rules. Those patients with higher priority data (e.g. worse health condition) will be presented first or in a more prominent position in the graphical dashboard (e.g. at the top, or in a special frame or window, or may otherwise be highlighted, e.g. with an overlay or similar).

By way of example, in FIG. 26, the patients are ordered in priority based on most recent worsening of the answer data relative to the patient's baseline.

In some configurations, the healthcare provider can configure the dashboard to pin or lock the position of the summary graph for one or more patients in place or in a particular desired position in the dashboard. For example, the healthcare provider may pin data relating to patients prone to exacerbation in a prominent or high-priority location within the graphical dashboard, relative to the data of the other patients.

In some configurations, the healthcare provider can configure the dashboard to select one or more different sections, groups or elements of the enquiry data for display in the one or more patient summary time-series charts. For example, a healthcare provider can customise or select a subset of the enquiry data relating to one or more desired health parameters (e.g., specific symptoms, specific medication, or other answer data) for display in the summary time-series charts (e.g., time-series graph and/or colour heatmap). In one embodiment, the dashboard of the data server is operable by a healthcare provider to select one or more sections (e.g. data rows) of the heatmap for display in the patient summary time-series charts. The customised summary time-series charts for multiple patients may be presented on a main page or homepage of the healthcare provider's dashboard. In one embodiment, the dashboard is operable to enable the healthcare provider to customise or select which sections or subsets of data to display in the summary time-series charts for each patient, i.e. the summary time-series charts displayed may be different for each patient, depending on the healthcare providers preferences or configurations.

The graphical dashboard provides a ‘lower resolution’ or summary data set for each patient the healthcare provider is monitoring, and enables the healthcare provider to easily scroll, search, traverse and/or review the summary data quickly to get a sense of any desired actions that might be required for each patient and/or to triage each patient as to priority, without needing to download or retrieve the full data reports in the first instance, thereby reducing data bandwidth, memory and/or processing required initially.

In one configuration, the summary data set for each patient may be interactive in that a healthcare provider may interact with the graphical user interface (GUI) presenting the dashboard, e.g. by selecting with a mouse click or touchscreen input upon a patient summary extract, which then pulls up or links to a new screen depicting a more detailed or full data report on the patient, i.e. a ‘higher resolution’ data set with more information, so the healthcare provider can jump or dive into more detailed data to make further decisions on patient treatment. For example, the dashboard may only display the time-series graph for each patient, as shown in FIG. 26, but clicking or selecting that graph for further interaction may pull up or switch to a new display screen with a full data report that includes the time-series graph presented alongside the colour heatmap, as per the data report shown in FIG. 22.

### 3.15 Graphical Rendering Engine for GUI or Display Device

In some embodiments, the data reports, time-series charts, and/or graphical dashboard may be rendered or displayed on the display device 512 of a user by a graphical rendering engine. This rendering engine may be performed or implemented in the data server 506, locally on the display device 512 and/or breathing assistance apparatus 502, or functions of the rendering engine may be spread across devices.

The display device 512 may be any electronic device with a display screen and data communication and access to the patient device and management platform 506. The graphical rendering engine may be performed entirely by a single processor or device, or spread across multiple devices in data communication. As discussed above, in one configuration, the graphics rendering engine may operate on data server 506, and output data reports, time-series charts, and/or the graphical dashboard for access via web browser on a web-platform or via another software application. In another configuration, the display device 512 or breathing assistance apparatus 502 may comprise a local graphical rendering engine that is configured to carry out the processing and rendering functions for raw data, pre-processed and coded data, and/or pre-compiled data reports, time-series charts, and/or graphical dashboard. In such configurations, the data may be locally cached and processed on the display device 512 or breathing assistance apparatus 502, for example.

The rendering engine receives the raw data and/or processed data from the data server and generates the data reports, time-series charts, and/or graphical dashboard in accordance with rendering instructions. The rendering instructions determine how the processed data is interpreted and/or decoded, and then presented and/or laid-out on the display screen, including format and positioning.

In some configurations, the rendering engine may process raw data into the data reports, time-series charts, and/or graphical dashboard, according to rendering instructions.

In some configurations, the rendering engine may render pre-processed and/or coded data into the data reports, time-series charts, and/or graphical dashboard, according to rendering instructions.

In some configurations, the rendering engine may render pre-compiled data reports, time-series charts, and/or graphical dashboards, according to rendering instructions.

3.16 Clinician Interactivity with Data Reports and/or Actions in View of Reports

As described above, the healthcare provider or other user accessing the data reports, time-series charts and/or graphical dashboard for display may interact with the displayed data in a number of ways.

In one operation, the user may use a zoom function to zoom in and out of the data.

In another operation, the user may use a click-through or linking function that providers click-through interaction in which the user can click or select elements or items of presented data, which then automatically links or jumps to related or associated data, including cascading between higher and lower-level data, or switching between graphs and plots, some examples of which are described above.

As shown in FIGS. 22-26, the answer data may be plotted in a graphical format that visually illustrates the health parameters recorded by the patient. In one embodiment the plotted health parameters allow a health provider to more easily review the health status of the user (patient). Thus, the plotted data (i.e. visual presentation of the processed responses to the enquiry relative to baseline data) allows a healthcare provider to track a change in the health parameters and determine a health condition or change in the health condition of the patient.

The answers given by the patient to the enquiry and/or the generated data reports may be used by a healthcare provider to determine a change in a health parameter (e.g., worse, same, better) of the patient.

In some embodiments, based on the answers given by a patient to one or more enquiries and/or the generated data reports, the server may alert a computing device (e.g., smartphone, tablet, wearable, workstation) operated by a healthcare provider.

In some embodiments a healthcare provider has access to the patient's answers or the generated data reports, and is able to use those answers and/or data reports, whether from a single session or multiple sessions of answers to enquires, to make decisions on doing any one or more of the following:
Change the patient's treatment prescription provided by the breathing assistance apparatus.
Customize the enquiries presented to that patient.
Prescribe a pharmacological intervention.
Provide or push messages or notifications to the breathing assistance apparatus and/or a patient's linked electronic device (e.g. Smartphone).
Trigger medical intervention, e.g. sending an ambulance to the patient.
Contacting the patient to check-in, e.g. by phone, email, digital message, and/or in-person visit.

In some embodiments, based on the patient's answers to the enquiries and/or generated data reports, from a single session or multiple sessions, the apparatus and/or the patient and device management platform is configured to contact a healthcare provider (for example a doctor) or alert an emergency service (for example an ambulance service).

In some embodiments, based on the determination that the patient's condition is getting worse, the apparatus and/or the patient and device management platform is configured to contact a healthcare provider (e.g. a doctor) or alert an emergency service (e.g. an ambulance service).

The healthcare provider may base their action on the plotted results of the patient's health condition and/or generated data reports.

The healthcare provider may receive the answers to the queries provided by the patient and/or generated data reports, and upon reviewing those answers and/or data reports may remotely change one or more treatment settings (the prescription) of the breathing assistance apparatus. Such as, the flow rate, percentage O2, treatment pressure, and/or duration of treatment.

The healthcare provider may customize the queries that are provided to the patient. For example, the queries may be customised to more effectively provide health care parameters targeted to their health needs, or the queries may be varied to maintain the patient's interest.

Based on the answers provided by the patient to the queries and/or generated data reports, the healthcare provider may prescribe pharmacological interventions such as prescribing antibiotics, steroids and/or inhaler use.

In some embodiments the queries may change as the queries asked may be dependent on the answers given previously (in the same session or in previous sessions). For example, the controller may be configured to selectively present particular queries based on the previous responses of the patient or based on a change in one or more health parameters over time or a change in the health condition of the patient over time.

The healthcare provider may determine if a patient's health condition is going to deteriorate based on the plotted results of the patient's medical condition and/or generated data reports. Alternatively, the change in a patient's health condition may be presented on the screen of the device or an alarm may be generated by the breathing assistance apparatus to indicate to a user that the patient needs to visit a doctor or hospital.

If the patent is particularly unwell, as indicated by a high or non-desirable score to the enquiry or based on the processed answer data or generated data reports, then that data may be sent with an alert, and/or sent to a healthcare provider's mobile device for urgent attention.

The present disclosure enables an engaging, easy-to-use enquiry that is displayed on the touch screen of a breathing assistance apparatus, along with subsequent data processing into data reports for rendering on a display screen for viewing by a healthcare provider.

To enable timely and informed use of the responses to the enquiry, it is helpful for the responses to be processed either locally on the breathing assistance apparatus or remotely, and then made available in a useable format. For example, the enquiry can be presented on the integrated touchscreen upon start-up of the breathing assistance apparatus before any other selectable options are presented to the user.

The presented enquires may be intuitive to engage the user and capture the user's attention. Since the patient is already accustomed to using the breathing assistance apparatus for receiving therapy, the patient is more likely to complete the answers to the queries of the enquiry. Further, making the enquiry intuitive and engaging encourages the patient to regularly interact with the enquiry, while ensuring that the enquiry is not overly tedious to complete.

The enquiry being presented on the integrated touchscreen of the apparatus also makes the enquiry easier to access since the user does not need to start or handle a second device (e.g. a phone). This makes the user more likely to complete the enquiry due to easier use (i.e. a single device for therapy and answering queries).

3.17 Patient Access and Review of Data Reports and/or Time-Series Charts

In some embodiments, as discussed, the patient may be able to access the data server (e.g. patient device and management platform) through a patient portal or similar to access their own processed answer data, data reports and/or time-series charts explained above, or a version of such data, reports or charts.

In some embodiments, the patients or user may additionally or alternatively view their processed answer data, data reports and/or time-series charts explained above. For example, at the end of the personal health enquiry, the breathing assistance apparatus may locally process the answer data or aspects of the answer data relative to stored or retrieve baseline data (similar to the data processing at the data server), and may be able to present on the display of the breathing assistance apparatus a data report and/or one or more of the time-series charts discussed above (e.g. colour heatmap and/or time-series graph of the category deviation parameters).

The patient or user may be able to navigate to such data reports and/or charts via menu screens or drop-down menus on the display, or data report graphical elements or touch buttons presented on the GUI of the touchscreen or via other physical buttons or user input elements provided near or adjacent the touchscreen display. In one configuration, an updated patient data report and/or time-series chart may automatically pop-up on the touchscreen display at the completion of each personal health enquiry.

In alternative configurations, the breathing assistance apparatus may retrieve the data reports and/or time-series charts from the data server for display on the touchscreen of the apparatus, rather than locally processing the answer data to generate the data reports and/or time-series charts.

In some embodiments, the breathing assistance apparatus and/or data server can be configured to send the patient data report and/or time-series charts to a patient email address and/or any number of nominated email addresses. The system may be configured to send emails in response to a received data request, automatically on a periodic basis, and/or on an adhoc basis, for example a healthcare provider may manually trigger the processed data to be emailed to the patient or other nominated email addresses.

In some embodiments, the breathing assistance apparatus and/or data server can be configured to send or push the patient data report and/or time-series charts directly to a patient's (or any one or more other nominated persons) smartphone or tablet or wearable device and/or other portable electronic device for display. For example, the patient's smartphone or portable electronic device may run a software application that is configured to receive or retrieve updated data reports and/or time-series charts from the breathing assistance apparatus and/or data server. The system may be configured to send the processed data in response to a received data request from the software application, automatically on a periodic basis, and/or on an adhoc basis, for example a healthcare provider may manually trigger the processed data to be pushed to the device(s) of the patient and/or other nominated persons.

### 3.18 Custom and Configurable Notifications

In some embodiments, the system disclosed may be configured to provide a healthcare provider and/or patient with custom and/or configurable notifications that are triggered based at least partly on the processing of the enquiry data received from the patient health enquiry and configurable trigger criteria and/or conditions. For example, one or more notifications may be configured to be sent to the healthcare provider if respective configurable triggering criteria are satisfied.

A healthcare provider is typically monitoring many patients, and the ability to customise and configure specific notifications for individual patients and/or groups or classes of patients with similar profiles, may increase the efficiency of patient monitoring and assessing patients, in some applications.

### Notifications

The term ‘notifications’ is intended to cover any electronic notification, alert, message, or alarm. The notifications may be visual, audible and/or tactile. Visual notifications may include text, imagery, symbols, icons, graphics, animations or any combination thereof for conveying the desired notification information. Audible notifications may include sounds, speech or any other suitable audio for conveying the desired notification information.

The notifications can be displayed or presented on the breathing assistance apparatus, a user interface of the data server (e.g., patient and device management platform), and/or sent or pushed to an electronic device of the healthcare provider and/or patient (e.g., smartphone, tablet, PC, wearable, or the like).

The processing of the enquiry data to trigger the notifications based on the respective trigger criteria may be carried out on the breathing assistance apparatus, data server (e.g., patient and device management platform), and/or another connected or linked electronic device in data communication with either the breathing assistance apparatus and/or data server.

### Configuration of Notifications

The process of configuring one or more notifications for a patient and/or patient group may be automatic, semi-automatic, and/or manual, as will be explained further in the following.

In an embodiment, each triggerable notification may be defined by a notification profile. The notification profile may comprise or have associated notification configuration data that comprises or defines the notification message data and the trigger criteria data. Either or both of the notification message and trigger criteria may be configurable or customisable.

The notification message data defines the message, content, or information to be conveyed in the notification. The trigger criteria data defines the trigger criteria or conditions that must be satisfied when processing the enquiry data in order to trigger the notification.

In an embodiment, one or more of the notifications may be automatically configured. For example, one or more default notification profiles may be loaded or assigned automatically for each patient based on the patient's profile and/or whether they belong to a specific patient group. These default notification profiles will cause default notifications to be triggered if the patient's enquiry data and/or other data satisfy the default trigger criteria.

In an embodiment, one or more of the notifications may be configured semi-automatically. For example, one or more default notification profiles may be loaded or assigned automatically for each patient as above based on the patient's profile and/or whether they belong to a specific patient group. The healthcare provider may then alter, adjust or re-configure any one or more of the loaded default notification profiles as desired. For example, the healthcare provider may delete or disable one or more of the default notifications, or they may alter, adjust or re-configured one or more aspects of the notification message data and/or trigger criteria data of one or more of the default notifications.

In an embodiment, one or more of the notifications may be configured manually by the healthcare provider for a patient. For example, the healthcare provider may be presented with a range or set of default notifications for selection, and may select one or more of these default notifications to apply to the patient. In one configuration, the system may suggest one or more notifications that may be applicable or recommended for the patient based on their patient profile and/or any patient group they may belong to. The healthcare provider may also modify, alter or adjust any of the selected default notifications, to further customise the notifications for a specific patient. In particular, the healthcare provider may modify any one or more aspects of the notification message data and/or trigger criteria data for each selected default notification. Additionally or alternatively, the system may provide an interface for the healthcare provider to generate and configure new notifications with custom notification message data and trigger criteria.

In one configuration, the system may provide the healthcare provider with all notification configuration options or modes described above, i.e., automatic, semi-automatic, and manual configuration options for the notifications are provided via a user interface (e.g., GUI or dashboard interface of the patient and device management platform). In another configuration, the system may restrict the notification configuration options to any one or more of the automatic, semi-automatic, and/or manual options or modes.

### Individual Patient Notifications

In an embodiment, the system is operable by a healthcare provider to create, modify, configure, delete and/or add individual patient notifications for any specific patient via the user interface. For example, the healthcare provider may tweak or modify the settings (e.g. notification message and/or trigger criteria) associated with any one or more of the existing notifications associated with a patient, and may create or generate new individual notifications for the patient.

### Patient Groups and Patient Group Notifications

As discussed previously, the patient and device management platform may store a patient profile for each patient. The patient profile may store various data about the patient including, but not limited to, patient details (e.g. name, address), demographic details (age, date of birth, gender), health conditions or history, treatment history, baseline data for the patient health enquiry, prescription data or therapy settings, patient unique identifier or ID, serial number or device ID for the patient's breathing assistance apparatus, or the like.

In an embodiment, the system may be provided or configured with one or more default patient groups, each defined by a patient group profile. Each patient group profile defines one or more group criteria that defines or classifies which patients fall within the patient group. For example, a patient will be linked or assigned to a patient group if the data from their patient profile matches or satisfies the group criteria for a patient group. The patient may be linked or associated with one or more defined patient groups, i.e. they may be a member of one or more patient groups.

In an embodiment, the system is configured to enable a healthcare provider to generate or create new patient groups, or modify the group criteria of existing patient groups. For example, the healthcare provider may define a new group and the associated group criteria based on any one or more of the data elements or categories contained within a patient profile. This enables the healthcare provider to generate custom patient groups that capture patients having specific combinations of similar patient profile data.

In an embodiment, each patient group may be configured with one or more patient group notifications. These patient group notifications each have a notification profile with notification configuration data as described earlier that defines the notification message and trigger criteria for the notification. As with the individual patient notifications, user interface of the system may allow a healthcare provider to create, modify, delete, configure and/or add patient group notifications for any one or more patient groups.

The system may be provided with one or more default patient group notifications for any one or more default patient groups. A healthcare provider may be able to modify or adjust any aspect of the default patient group notifications (e.g., the notification message and/or trigger criteria). The system may also be operable to enable a healthcare provider to generate new patient group notifications for any existing default patient groups and/or newly created patient groups, in which the healthcare provider is able to configure and customise the notification message and trigger criteria.

As discussed above, one or more notifications or notification profiles may be linked or assigned to a patient depending on which patient group or groups they belong to, if any. Each patient may have linked notifications comprising individual patient notifications and/or patient group notifications for any patient group they belong to. In this example embodiment, the system enables a healthcare provider to create new custom patient groups and/or modify and create new associated patient group notifications. This means a healthcare provider may modify the configuration of a patient group notification, and this change is rolled-out or globally updated for all patients in the relevant patient group, which is an efficient mechanism to update and customise notification settings the healthcare provider's patients.

By way of example, a healthcare provider may to create a patient group defined by the following group criteria: age (e.g., 80-84 years), gender (e.g., male), smoker. The healthcare provider may have knowledge that patients fitting this profile are prone to exacerbation if three specific health parameters (e.g., cough, sputum production, and tiredness) worsen by a certain degree. The healthcare provider is then able to configure a specific patient group notification for this group of patient that triggers based on trigger criteria specifying a worsening of the mentioned health parameters to a specified degree. The system is then configured to compare the incoming enquiry data for each of the patients fitting the patient group profile to the trigger criteria, and triggers a notification to the healthcare provider if any of the patients in the group appear vulnerable to exacerbation with their enquiry data satisfying the trigger criteria.

### Trigger Criteria for Notifications

Each notification (whether individual and/or patient group) has default and/or configurable trigger criteria. If these trigger criteria are satisfied by the enquiry data and/or other data for the patient, the notification is triggered. In some examples, the trigger criteria are also based on the patient's baseline data.

The trigger criteria for each notification may require the enquiry data for the patient and/or other related patient data (e.g. baseline data) to satisfy any one or more criteria or conditions. The criteria or conditions may compare one or more health parameters or elements or data sets of the enquiry data and/or deviation data (relative to the patient's baseline data) to one or more thresholds, maximum thresholds, minimum thresholds, range thresholds, trend parameters or thresholds or the like. The criteria or conditions may also include associated time period thresholds or parameters. The trigger criteria may compare the enquiry data to absolute thresholds, and/or may compare the enquiry data to the patient's baseline data.

To illustrate the trigger criteria further, some examples of trigger criteria are provided in the following:
Trigger notification if health parameter x and y get worse for patient, where x and y may be selected from any of the symptom and/or medication health parameters captured in the enquiry data—examples of which are displayed and described in FIGS. 22 and 23.
Trigger notification if all health parameters get worse for patient.
Trigger notification if health parameter z gets worse and stays worse for a specified time period (e.g., four consecutive days) for patient, z may be selected from any of the symptom and/or medication health parameters captured in the enquiry data-examples of which are displayed and described in FIGS. 22 and 23.
Trigger notification if 5 or more health parameters get worse for the patient.
Trigger notification if health parameter x deviates from its baseline by 3 levels for the patient.
Trigger notification is health parameter x equals 4 and health parameter y deviates negatively/adversely from its baseline.

The trigger criteria may further specify what qualifies as ‘worse’. In one example, this may simply mean deviating negatively from the patient's baseline by any level, or by a certain number of levels. In another example, this may simply mean the rating or score getting worse compared to the previous questionnaire by any level, or by a certain number of levels on the rating or scale.

As discussed above, some trigger criteria may be based on comparing the enquiry data for one or more selected health parameters to one or more absolute thresholds, agnostic of the patient's baseline data. Other trigger criteria may be based on comparing the enquiry data to thresholds based on the baseline data. For example, the trigger criteria may be based on comparing the deviation data for a health parameter (e.g. how much the health parameter score, rating or answer deviates from the patient's baseline) to one or more thresholds.

In some examples, the trigger criteria for one or more of the notification profiles may additionally or alternatively be at least partly based on or a function of the patient physiological parameter data, therapy setting data, and/or gases flow data described previously. For example, such data may be compared to one or more thresholds or conditions as part of the trigger criteria.

### Additional Notification Settings—Presentation and Recipients

In some embodiments, the notification profiles may further comprise data or configuration data or settings that define the nature of the presentation of the notification and/or intended recipients or recipient devices of the notification. A healthcare provider may create or adjust these additional settings for each notification profile via the user interface, in a similar manner to configuring the notification message and trigger criteria.

In one example, the presentation or format of each notification may be configured or defined by presentation data associated with the notification profile. For example, some or all of the notifications may provide one or more selectable presentation options for the notification, such as visual presentation (e.g. as a message or alert on a display), audible presentation (e.g. via a speaker of a device), and/or tactile (e.g. via a vibration component of the device). The nature of the presentation may also be customised or configured to depend or be a function of the intended recipient or recipient device that presents the notification.

In another example, the recipient data of each notification may be configured or defined. The recipient data may define the intended recipient or recipient device that the triggered notification is sent or pushed to. For example, the recipient data may define that the notification is to be sent or pushed to any one or more of the patient, healthcare provider, and/or one or more other third-party recipients. Additionally or alternatively, the recipient data may define one or more specific recipient devices or systems to send or push the notification to. For example, the recipient data may be configured to selectively send or push the triggered notification to any one or more of the breathing assistance apparatus, data server, and/or any electronic device associated or in data communication with the breathing assistance apparatus and/or data server (e.g., a smartphone, tablet, PC, wearable or other electronic device of the patient and/or healthcare provider.

Patient Notified that Healthcare Provider Alerted

In some configurations, the notifications can be configured to alert the patient as to when a notification has been sent to their healthcare provider. For example, as discussed above, notifications may be configured to notify the healthcare provider if their associated trigger criteria are satisfied. These healthcare provider notifications can also be optionally configured to alert or notify the patient that their healthcare provider has been notified or alerted. For example, when a triggered notification is sent or presented to their healthcare provider, a notification, message or alert indicating this may be displayed or presented on the breathing assistance apparatus (e.g., on its display and/or audibly via a speaker of the apparatus). Such a configuration may be useful in re-assuring a patient that their doctor or healthcare provider has been alerted of their worsening condition for example.

### 3.19 Pre-Programmed or Customisable Trigger Thresholds or Conditions

Any of the trigger thresholds or trigger criteria described above in relation to any aspect or action may be pre-programmed or customisable. For example, trigger thresholds or trigger criteria may be associated with triggering, changing, and/or causing any one or more of the following: notifications, messages, prompts, presentation of the personal health enquiry, presentation of specified queries or follow-on queries or conditional queries, frequency of presentation of the personal health enquiry, transmission of data for processing. Any of the trigger thresholds, criteria or conditions associated with these example aspects may be pre-preprogrammed or configurable by a patient and/or healthcare provider, e.g., via a user interface of the breathing assistance apparatus and/or data server (e.g., patient and device management platform), or a user interface associated with an electronic device in data communication with the breathing assistance apparatus or data server. By way of example only, any of the trigger thresholds, criteria or conditions may be at least partly based on or a function of any one or more of the following: enquiry data, answer data, skipped data, baseline data, deviation data, hospital stay data, patient physiological parameter data, therapy setting data, gases flow data, sensor data, and/or any other data or parameters discussed.

### 4. Terminology and Definitions

The phrase ‘colour spectrum’ as used in this specification and claims is intended to mean, unless the context suggests otherwise, a spectrum, band, scale or continuum of colours or shades, typically having an associated base or dominant colour with the spectrum comprising a plurality or range of colour values of the base or dominant colour that vary across the spectrum in one or more colour or spectral characteristics such as, but not limited to, shade, brightness, tone, tint, wavelength, and/or frequency, and wherein the base or dominant colour may be any one or a combination of the following: a primary colour; any mixture of primary colours; traditional colours such as red, orange, yellow, green, cyan, blue, and violet; black; white; and/or grey, and in some embodiments the colour spectrums may be considered or perceived as ‘shade spectrums’, and in some embodiments a colour spectrum may represent or comprise a spectrum, band, scale or continuum of changing indicia (e.g. text, numeral, icon, symbol or similar) in which the indicia may change in size, shape, or other aspect depending on improvement or worsening of the answer, or a combination of any such spectrums above.

The term ‘heatmap’ as used in this specification and claims is intended to mean, unless the context suggests otherwise, is intended to mean a representation of data in the form of a diagram or chart or graphic in which the values or data are represented at least partially by colours or colour values. In one illustrated example shown in FIG. 23 for example, the heatmap comprises the data represented as colours or coloured data cells over a time scale or time-series.

The phrase ‘patient and device management platform’ as used in this specification and claims is intended to mean, unless the context suggests otherwise, any electronic or software platform, system, portal, or interface that a patient, healthcare provider, device manufacturer, insurer, and/or any other authorised party, may access to perform tasks or functions such as, but not limited to, any one or more of the following: reviewing and interacting with patient data or information, including usage data, compliance data, health enquiry data, and/or data reports; reviewing and interacting with breathing assistant device treatment settings and/or configurations, and/or fault and diagnostic information; and/or other general or specific data processing, filtering, analysis, storage, reporting, downloading, uploading, transmission, and typically, but necessarily, such an electronic or software platform, system, portal or interface being hosted by a data server (e.g. Cloud-server and/or website server) and accessible via a software application such as a website interface on a website browser application, and/or via a software application in a host-client type system architecture.

The phrase ‘healthcare provider’ as used in this specification and claims is intended to include, unless the context suggests otherwise, any party that provides healthcare, such as a hospital system, physician, clinician, doctor, medical consultant, or any other healthcare professional.

The phrase ‘health parameter’ as used in this specification and claims is intended to mean, unless the context suggests otherwise, a measurable (objective or subjective) factor that relates to a patient's mental and/or physical condition. In some embodiments the health parameter can be a subjective factor related to a patient's perception of their health. For example, this may be a patient's general feeling.

The phrase ‘health condition’ as used in this specification and claims is intended to mean, unless the context suggests otherwise, a collective determination of the patient's health based on the health parameters of the patient.

The phrase ‘data server’ as used in this specification and claims is intended to mean, unless the context suggests otherwise, any type or form of server, server system, data service, network of servers, server platform or server service, including remote or cloud-based platforms, systems and servers, such as those that typically provide for data processing, communication, hosting, service or server architectures and/or data storage services and functionality, whether proprietary systems, third party systems and services, or a combination.

The phrases ‘computer-readable medium’ or ‘machine-readable medium’ as used in this specification and claims should be taken to include, unless the context suggests otherwise, a single medium or multiple media. Examples of multiple media include a centralised or distributed database and/or associated caches. These multiple media store the one or more sets of computer executable instructions. The phrases ‘computer-readable medium’ or ‘machine-readable medium’ should also be taken to include any medium that is capable of storing, encoding or carrying a set of instructions for execution by a processor of a computing device and that cause the processor to perform any one or more of the methods described herein. The computer-readable medium is also capable of storing, encoding or carrying data structures used by or associated with these sets of instructions. The phrases ‘computer-readable medium’ and ‘machine readable medium’ include, but are not limited to, portable to fixed storage devices, solid-state memories, optical media or optical storage devices, magnetic media, and/or various other mediums capable of storing, containing or carrying instruction(s) and/or data. The ‘computer-readable medium’ or ‘machine-readable medium’ may be non-transitory.

The term ‘comprising’ as used in this specification and claims means ‘consisting at least in part of’ or ‘including, but not limited to’ such that it is to be construed in an inclusive sense as opposed to an exclusive or exhaustive sense. When interpreting each statement in this specification and claims that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term ‘and/or’ means ‘and’ or ‘or’, or both.

The use of ‘(s)’ following a noun means the plural and/or singular forms of the noun.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

### 5. General

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

In the above description, specific details are given to provide a thorough understanding of the embodiments. However, it will be understood by one of ordinary skill in the art that the embodiments may be practiced without these specific details. For example, software modules, functions, circuits, etc., may be shown in block diagrams in order not to obscure the embodiments in unnecessary detail. In other instances, well-known modules, structures and techniques may not be shown in detail in order not to obscure the embodiments.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc., in a computer program. When a process corresponds to a function, its termination corresponds to a return of the function to the calling function or a main function.

Aspects of the systems and methods described above may be operable on any type of general purpose computer system or computing device, including, but not limited to, a desktop, laptop, notebook, tablet, smart television, gaming console, or mobile device. The term “mobile device” includes, but is not limited to, a wireless device, a mobile phone, a smart phone, a mobile communication device, a user communication device, personal digital assistant, mobile hand-held computer, a laptop computer, wearable electronic devices such as smart watches and head-mounted devices, an electronic book reader and reading devices capable of reading electronic contents and/or other types of mobile devices typically carried by individuals and/or having some form of communication capabilities (e.g., wireless, infrared, short-range radio, cellular etc.).

Aspects of the systems and methods described above may be operable or implemented on any type of specific-purpose or special computer, or any machine or computer or server or electronic device with a microprocessor, processor, microcontroller, programmable controller, or the like, or a cloud-based platform or other network of processors and/or servers, whether local or remote, or any combination of such devices.

Furthermore, embodiments may be implemented by hardware, software, firmware, middleware, microcode, or any combination thereof. When implemented in software, firmware, middleware or microcode, the program code or code segments to perform the necessary tasks may be stored in a machine-readable medium such as a storage medium or other storage(s). A processor may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the above description, a storage medium may represent one or more devices for storing data, including read-only memory (ROM), random access memory (RAM), magnetic disk storage mediums, optical storage mediums, flash memory devices and/or other machine or computer readable mediums for storing information.

The various illustrative logical blocks, modules, circuits, elements, and/or components described in connection with the examples disclosed herein may be implemented or performed with a general purpose processor, a digital signal processor (DSP), an application specific integrated circuit (ASIC), a field programmable gate array (FPGA) or other programmable logic component, discrete gate or transistor logic, discrete hardware components, or any combination thereof designed to perform the functions described herein. A general purpose processor may be a microprocessor, but in the alternative, the processor may be any conventional processor, controller, microcontroller, circuit, and/or state machine. A processor may also be implemented as a combination of computing components, e.g., a combination of a DSP and a microprocessor, a number of microprocessors, one or more microprocessors in conjunction with a DSP core, or any other such configuration.

The methods or algorithms described in connection with the examples disclosed herein may be embodied directly in hardware, in a software module executable by a processor, or in a combination of both, in the form of processing unit, programming instructions, or other directions, and may be contained in a single device or distributed across multiple devices. A software module may reside in RAM memory, flash memory, ROM memory, EPROM memory, EEPROM memory, registers, hard disk, a removable disk, a CD-ROM, or any other form of storage medium known in the art. A storage medium may be coupled to the processor such that the processor can read information from, and write information to, the storage medium. In the alternative, the storage medium may be integral to the processor.

One or more of the components and functions illustrated the figures may be rearranged and/or combined into a single component or embodied in several components without departing from the scope of the disclosure. Additional elements or components may also be added without departing from the scope of the disclosure. Additionally, the features described herein may be implemented in software, hardware, as a business method, and/or combination thereof.

In its various aspects, embodiments of the disclosure can be embodied in a computer-implemented process, a machine (such as an electronic device, or a general purpose computer or other device that provides a platform on which computer programs can be executed), processes performed by these machines, or an article of manufacture. Such articles can include a computer program product or digital information product in which a computer readable storage medium containing computer program instructions or computer readable data stored thereon, and processes and machines that create and use these articles of manufacture.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this disclosure may comprise, additional to its essential features described herein, one or more features as described herein from each other embodiment of the invention disclosed herein.

This disclosure may also be said broadly to consist in the parts, elements and features referred to or indicated in this disclosure, individually or collectively, and any or all combinations of any two or more said parts, elements or features, and where specific integers are mentioned herein which have known equivalents in the art to which this disclosure relates, such known equivalents are deemed to be incorporated herein as if individually set forth.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

The scope of the present disclosure is not intended to be limited by the specific disclosures of embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1-20. (canceled)

21. A system comprising:
multiple respiratory therapy devices that are configured to deliver a flow of gases to respective multiple users for respiratory therapy, each respiratory therapy device comprising:
a flow generator for generating the flow of gases,
a humidifier for heating and humidifying the flow of gases,
a controller that is operable to control the flow generator and humidifier,
a user interface comprising a display to enable user interaction with the controller;
a data server that is in data communication with the respiratory therapy devices; and
a display device that is in data communication with the data server,
the controllers of the respiratory therapy devices being configured to receive answer data comprising symptom data and/or medication data based on user input to the user interface to a set of presented queries on the display relating to one or more health parameters from at least one of a symptoms category and a medication category, and send the answer data to the data server,
the data server being configured to receive the answer data from the multiple respiratory therapy devices for the multiple users, process the answer data relative to baseline data associated with each respective user to generate a graphical dashboard that is accessible or rendered for display by the display device, and wherein:
the graphical dashboard depicts a summary time-series chart for each user of their processed answer data relative to their baseline data on a category basis, each summary time-series chart depicting or representing deviations of the symptom data and/or medication data relative to their respective baseline data over a time period, and
the generated graphical dashboard being displayed or rendered on the display device;
wherein the summary time-series chart for each user comprises a time-series graph that plots both symptom data deviation and medication data deviation and the data server is configured to process the answer data to extract or generate symptom data deviation and medication data deviation in a form of category deviation parameters for plotting that represent a total number of health parameters in each respective category for a user that have deviated from their respective baseline data for each time-interval associated with a time-axis of the time-series graph.

22. (canceled)

23. The system according to claim 21, wherein the plots of symptom data deviation and medication data deviation are visually distinguishable from each other or visually represented as separate data sets within the same time-series graph.

24. The system according to claim 23, wherein the plots of symptom data deviation and medication data deviation are represented by the same or different types of plots.

25. The system according to claim 23, wherein the plots of symptom data deviation and medication data deviation are represented by independent plots within the same time-series graph or as combined plots within the same time-series graph.

26. The system according to claim 21, wherein the plots representing the symptom data deviation and medication data deviation are selected from any one or more of the following types of plots: dot plot, line chart or line graph, bar chart, lollipop chart, stacked bar chart, and grouped bar chart.

27. The system according to claim 21, wherein the summary time-series graph for each user comprises the symptom data deviation being plotted as a time-series bar chart and the medication data deviation being plotted as a time-series line chart, or vice versa, within the same time-series graph.

28. The system according to claim 27, wherein the bar and line charts representing the symptom data deviation and medication data deviation are visually distinguished from each other by colour, pattern, or shade.

29. (canceled)

30. The system according to claim 21, wherein the data server is configured to generate:
a symptom category deviation parameter representing or being a function of the total number of health parameters that deviate from their baseline for each time-interval, and a medication category deviation parameter representing or being a function of the total number of health parameters that deviate from their baseline for each time-interval, each deviation parameter being plotted in the time-series graph; or
a worsening deviation parameter for each category representing or being a function of the total number of health parameters in the category that have a worsening deviation from their baseline for each time-interval, and an improvement deviation parameter for each category representing or being a function of the total number of health parameters in the category that have an improving deviation from their baseline for each time-interval, each deviation parameter being plotted in the time-series graph.

31. The system according to claim 30, wherein the data server is configured to generate a symptom category deviation parameter(s) and medication category deviation parameter(s) for each time-interval based on a data set representing a related colour-coded time-series plot of the answer data for the user for the one or more health parameters over a time period, wherein each plotted answer of the answer data is selectively colour-coded for display according to a baseline colour spectrum or one or more deviation colour spectrums, the colour spectrum selected for each answer being a function of or based on any deviation of the answer data relative to the baseline data.

32. The system according to claim 31, wherein the category deviation parameters for each time-interval are based on or are a function of the number of answers in each respective category that are colour coded into a respective one of the deviation colour spectrums.

33. The system according to claim 21, wherein the summary time-series chart for each user comprises a colour-coded time-series plot of the answer data for the user for the one or more health parameters over a time period, and wherein each plotted answer of the answer data is selectively colour-coded for display according to a baseline colour spectrum or one or more deviation colour spectrums, the colour spectrum selected for each answer being a function of or based on any deviation of the answer data relative to the baseline data.

34. The system according to claim 33, wherein the plotted answers are grouped in rows or otherwise aligned with their associated health parameter when plotted against a time-axis of the colour-coded time-series plot.

35. The system according to claim 33, wherein the plotted answers are presented in defined category groups based on the category associated with the health parameter to which the plotted answer relates.

36. The system according to claim 21, wherein the data server is further configured to process the answer data for the multiple users to determine a priority status between the users, and wherein the graphical dashboard generated is configured to display or present the summary time-series charts of the multiple users based at least partly on the determined priority status of the users.

37. The system according to claim 36, wherein the priority status of each user represents their general health status relative to other users based on the answer data, and wherein the graphical dashboard is configured such that the summary time-series charts are presented in an order or arrangement that prioritises the users having a worse health status according to their priority status.

38-79. (canceled)
